(19) United States
(12) Patent Application Publication
(10) Pub. No.: US 2024/0360456 A1
(43) Pub. Date: Oct. 31, 2024

(54) SYNTHETIC RNAS AND USES THEREOF

(71) Applicants: Arizona Board of Regents on Behalf of the University of Arizona, Tucson, AZ (US); THE UNIVERSITY OF SOUTHERN MISSISSIPPI, Hattiesburg, MS (US)

(72) Inventors: Judith K. Brown, Tucson, AZ (US); Mosharrof Hossain Mondal, Tucson, AZ (US); Jorge Rafael Paredes Montero, Tucson, AZ (US); Alex Flynt, Hattiesburg, MS (US)

(21) Appl. No.: 18/681,276

(22) PCT Filed: Aug. 8, 2022
(86) PCT No.: PCT/US2022/074648

§ 371 Date: Feb. 5, 2024

Related U.S. Application Data
(60) Provisional application No. 63/230,560, filed on Aug. 6, 2021.

(51) Int. Cl. C12N 15/113

(52) U.S. Cl. CPC C12N 15/1137; C12N 15/1138; C12Y 302/0102; C12N 2310/14

(57) ABSTRACT

Provided herein are synthetic RNAs comprising a piRNA sequence and a target sequence for a gene of interest. The synthetic RNAs find use in methods of gene editing, in particular in methods of silencing expression of the gene of interest.

### STATEMENT REGARDING RELATED APPLICATIONS

This application claims priority to U.S. Provisional Application No. 63/230,560, filed Aug. 6, 2021, the entire contents of which are incorporated herein by reference for all purposes.

### REFERENCE TO SEQUENCE LISTING

The contents of the electronic sequence listing (UAZ-39735-601.xml; Size: 133,000 bytes; and Date of Creation: Aug. 8, 2022) is herein incorporated by reference in its entirety.

### STATEMENT OF GOVERNMENT SUPPORT

This invention was made with government support under Grant No. 2018-70016-27411, awarded by the USDA/NIFA, and Grant No. 1845978, awarded by the NSF. The government has certain rights in the invention.

### FIELD OF THE DISCLOSURE

Provided herein are synthetic RNAs comprising a piRNA sequence and a target sequence for a gene of interest. The synthetic RNAs find use in methods of gene editing, in particular in methods of silencing expression of the gene of interest.

### BACKGROUND OF THE DISCLOSURE

The whitefly Bemisia tabaci (Genn.) (Aleyrodidae, Hemiptera) is considered a cryptic or sibling species. Although most B. tabaci are relatively benign, at least two variants/cryptic species transmit plant viruses and are among the most invasive species causing damage to crops grown in sub-tropical, tropical, and mild temperate parts of the world (Brown et al, 1995; Brown, 2010; Chen et al, 2016; de Moya et al, 2019; Grover et al, 2019). Chemical pesticides can be toxic to the environment and consumers of these products and regularly have become ineffective when resistance develops (Chen et al, 2016). B. tabaci is closely related to greenhouse and spiraling whiteflies, and several other related phloem-feeding pests/pathogen vectors, including aphids, mealybugs, and psyllids.

RNA interference (RNAi) technology has been shown to be applicable as a low-toxicity biopesticide to control agricultural insect pests and vectors of plant pathogens through silencing essential, biologically relevant genes (Zotti & Smagghe, 2015). RNAi shows great potential to be highly species specific and thereby spares beneficial organisms and is nontoxic to humans and other animal consumers. The RNAi approach for insect pest/vector control relies on the ingestion of long dsRNAs to trigger gene silencing via siRNA production after Dicer processing (Head et al, 2017; Knorr et al, 2018). Although a number of products are available, some arthropod pests exhibit moderate or only minor sensitivity to dsRNA upon ingestion (Yu et al, 2013; Zhu & Palli, 2020). Accordingly, what is needed are uniquely engineered RNAi triggers relevant to each target species (Shukla et al, 2016; Parsons et al, 2018).

### SUMMARY OF THE DISCLOSURE

In some aspects, provided herein are synthetic RNAs. The synthetic RNAs described herein find use in methods of gene editing, such as methods of gene silencing.

In some embodiments, provided herein is a synthetic RNA comprising a piRNA sequence, and a target sequence for a gene of interest. The synthetic RNA may induce silencing of the gene of interest. In some embodiments, silencing of the gene of interest is induced at least in part by ping-pong biogenesis of piRNAs. In some embodiments, silencing of the gene of interest is induced at least in part by biogenesis of piRNAs through piRNA phasing.

In some embodiments, the piRNA sequence comprises a left flanking sequence and a right flanking sequence. In some embodiments, the target sequence for the gene of interest is sandwiched in between the left flanking sequence and the right flanking sequence. The left flanking region and/or the right flanking region may be designed to permit ping-pong biogenesis of additional siRNAs. In some embodiments, the left flanking region and/or the right flanking region may be designed to permit biogenesis of additional siRNAs through piRNA phasing mechanisms.

In some embodiments, the left flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 4. In some embodiments, the left flanking region comprises a nucleotide sequence having at least 80% sequence identity with SEQ ID NO: 4. In some embodiments, the left flanking region comprises a nucleotide sequence having at least 90% sequence identity with SEQ ID NO: 4. In some embodiments, the left flanking region comprises the nucleotide sequence of SEQ ID NO: 4. In some embodiments, the right flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 5. In some embodiments, the right flanking region comprises a nucleotide sequence having at least 80% sequence identity with SEQ ID NO: 5. In some embodiments, the right flanking region comprises a nucleotide sequence having at least 90% sequence identity with SEQ ID NO: 5. In some embodiments, the right flanking region comprises the nucleotide sequence of SEQ ID NO: 5. In some embodiments, the left flanking region comprises a sequence having at least 50% identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 4 and the right flanking sequence comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 5.

In some embodiments, the left flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 6. In some embodiments, the left flanking region comprises a nucleotide sequence having at least 80% sequence identity with SEQ ID NO: 6. In some embodiments, the left flanking region comprises a nucleotide sequence having at least 90% sequence identity with SEQ ID NO: 6. In some embodiments, the left flanking region comprises the nucleotide sequence of SEQ ID NO: 6. In some embodiments, the right flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 7. In some embodiments, the right flanking region comprises a nucleotide sequence having at least 80% sequence identity with SEQ ID NO: 7. In some embodiments, the right flanking region comprises a nucleotide sequence having at least 90% sequence identity with SEQ ID NO: 7. In some embodiments, the right flanking region comprises the nucleotide sequence of SEQ ID NO: 7. In some embodiments, the left flanking region comprises a sequence having at least 50% identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 6 and the right flanking sequence comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 7.

In some embodiments, the left flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 71. In some embodiments, the right flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 72. In some embodiments, the left flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 71 and the right flanking region comprises a nucleotide sequence having at least 50% sequence identity with (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) SEQ ID NO: 72.

In some embodiments, the left flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 73. In some embodiments, the right flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 74. In some embodiments, the left flanking region comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) with SEQ ID NO: 73 and the right flanking region comprises a nucleotide sequence having at least 50% sequence identity with (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100% identity) SEQ ID NO: 74.

Any suitable gene of interest may be the target of the synthetic RNAs described herein. In some embodiments, the gene of interest is a gene in a hemipteran organism. In some embodiments, the gene of interest is the gene of interest is aquaporin (AQP1), alpha glucosidase 1 (AGLU1), v-ATPase-A, v-ATPase-B, v-ATPase-D, v-ATPase-E, Delta-24 sterol reductase (D-24), cholesterol desaturase (C7), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP-family verprolin-homologous protein (WAVE), or Actin related 2/3.

In some embodiments, the gene of interest is the aquaporin 1 gene (AQP1). In some embodiments, the target sequence for the gene of interest comprises at least 20 contiguous nucleotides present in SEQ ID NO: 2. In some embodiments, the target sequence for the gene of interest comprises at least 100 contiguous nucleotides present in SEQ ID NO: 2. In some embodiments, the target sequence for the gene of interest comprises the nucleotide sequence of SEQ ID NO: 2.

In some embodiments, the gene of interest is the alpha glucosidase 1 gene (AGLU1). In some embodiments, the target sequence for the gene of interest comprises at least 20 contiguous nucleotides present in SEQ ID NO: 3. In some embodiments, the target sequence for the gene of interest comprises at least 100 contiguous nucleotides present in SEQ ID NO: 3. In some embodiments, the target sequence for the gene of interest comprises the nucleotide sequence of SEQ ID NO: 3.

In some embodiments, the gene of interest is v-ATPase-D. In some embodiments, the target sequence for the gene of interest comprises at least 20 contiguous nucleotides present in SEQ ID NO: 70. In some embodiments, the target sequence comprises at least 100 contiguous nucleotides present in SEQ ID NO: 70.

Further provided herein are methods of silencing gene expression in an organism. The methods comprise providing to the organism a synthetic RNA as described herein. In some embodiments, the organism is an insect. In some embodiments, the organism is a hemipteran.

### BRIEF DESCRIPTION OF THE DRAWINGS

FIG. 1A-1D show RNAi pathways in whitefly (B. tabaci). (FIG. 1A) Relatedness of argonaute and Piwi (Ago/Piwi) proteins from whitefly (Bta) to orthologs in Drosophila (Dme), Tribolium (Tca), and select family members from C. elegans (Cel). Ago and Piwi clades highlighted by colored boxes and whitefly genes in red text. Phylogenetic tree was constructed using the maximum likelihood method. Branch support values shown at nodes. (FIG. 1B) Expression determined by RPKM of whitefly Ago/Piwi proteins in whole body, gut, and salivary gland. (FIG. 1C) Numbers of miRNAs annotated in this study. Loci are categorized into those conserved with Drosophila, novel highly confident, and lower confidence candidates. (FIG. 1D) Distribution of small RNA read sizes mapping to the whitefly genome (MEAM v1.2) and piRNA biogenesis modes. Left inset shows read overlap Z-scores to demonstrate the ping-pong piRNA signature of 10-nt overlaps, and right panel distance to trailing 1U reads showing the phasing signature. Bars in the size distribution are colored based on the portion of reads with the sequence identity indicated in the inset legend.

FIG. 2A-2E show whitefly small RNA expressing loci. (FIG. 2A) Comparison of 3,878 small RNA loci is annotated in whitefly by locus size, number of mapped reads, and the ratio of short (19-23 nt) to long (25-30 nt) mapping reads. (FIG. 2B) Visualization of small RNA sizes and piRNA biogenesis signatures for all 3,878 loci. Each row of the heat map represents a locus, which is arranged by read size bias with short read bias at the top and long bias at the bottom. Left panel shows size distribution. Nucleotide sizes are indicated below. Arrows at top show sizes expected to represent siRNAs (si) and piRNAs (pi). Middle panel shows read overlaps quantified by Z-score, arrow shows the 10-nt overlap size. Right panel shows distance of trailing 1U reads; arrow shows the 2-nt proximal read distance. Dashed line box highlights the ˜100 loci that do not have piRNA signatures in terms of read size, overlaps, or phasing. This group of loci have more reads at the 22 nt (siRNA) size. (FIG. 2B, FIG. 2C) Matrix of Dicer-2 nt overhang signature calculated for loci in the dashed box in panel (FIG. 2B). Read pairs where the query read overlapped by 2 minus its total length were quantified and plotted in the heat map. Line of boxes below the matrix show the read size distribution for reads mapping to the analyzed loci (dashed box in FIG. 2B). (FIG. 2D, FIG. 2E) Number of mRNA and transposable element targets for the 50 most high expressing (FIG. 2D) loci biased to long reads or (FIG. 2E) loci biased toward short reads.

FIG. 3A-3C show characterization of whitefly loci with Dicer cleavage signature. (FIG. 3A) Intersection of Dicer processing loci showing 2-nt overhangs for reads sized 20-23 nt with long and short read loci. The sequence biases of Dicer read loci are shown below in the seqlogo graphic. (FIG. 3B) Appearance of Dicer produced small RNAs (siRNAs) at sites of convergent transcription. Top panel shows expression of siRNAs in a piRNA cluster. Bottom panel is a cis-natural antisense transcript (cis-NAT). Blue trace shows all reads mapping to locus. Read trace shows reads with Dicer-2-nt overhang cleavage pattern. (FIG. 3C) Read size distribution and biogenesis pattern of small RNAs produced at 76 Dicer signature loci. Length of reads in heat maps is indicated below. Curated identities are shown on the left. The leftmost heat map shows the distribution of reads sizes, middle shows z-scores for 2-nt overhangs (siRNAs), and right heat maps show z-scores for 10-nt overlaps (piRNAs).

FIG. 4A-4E show metabolism of exogenous dsRNAs in whitefly. (FIG. 4A) Accumulation of reads mapping to dsRNA sites (green boxes) in the context of the originating transcript from potato psyllid (B. cockerelli). Blue trace shows all reads mapping to the locus. Read trace shows reads with Dicer cleavage pattern. (FIG. 4A, FIG. 4B) Size distribution of read derived from the three off-target dsRNAs (shown in (FIG. A)). Red arrow shows the expected size of siRNAs (22 nt). (FIG. 4C) Balloon plot showing characterization of sequence biases in exogenous siRNAs. Read sizes are indicated below. Color and diameter of circle scale with Z-scores quantifying different size reads. On left, the sequence identities of small RNA subsets are indicated for the first base of the read and the 20th base of the read. N=any residue, H=U/A/C, D=A/U/G, and W=A/U. The left group of balloons show the abundance of reads, and the right group of circles abundance of reads with 2-nt overhangs. (FIG. 4D, FIG. 4E) Differential expression of (FIG. 4D) small RNA loci and (FIG. 4E) mRNAs between whiteflies treated with water or the three off target dsRNAs. Data points colored by identity. Circles represent nonsignificant change in expression, triangles significant. Dashed circle shows location of Dicer and Ago proteins in the scatterplot.

FIG. 5A-5F show exogenous piRNA-mediated gene silencing in whitefly. (FIG. 5A) Relative expression of AQP1 and AGLU1 genes determined by qRT-PCR after feeding with synthetic RNAs generated from piRNA triggers. Blue bar graphs are results when target gene sequences are fused to sequence from piRNA-biased locus 6 (piRB-6) sequences. Green graphs are when they are fused to No Bias-14 sequences. At least three independent biological replicates were used for each type of feeding. Error bars show standard error, and letters indicate significance groups determined by Tukey's HSD test. *P≤0.05. (FIG. 5B, FIG. 5C, FIG. 5D, FIG. 5E, FIG. 5F) Analysis of small RNA-sequencing data from animals fed piRB-6-based piRNA triggers that map to the synthetic RNAs. (FIG. 5B) Portion of small RNA-sequencing reads with 1U residues shows biased to long (piRNA) sized reads. Black bars are from double-stranded (DS) triggers and gray from singlestranded (SS) versions. (FIG. 5C). Enrichment of ping-pong piRNA pairs in longer sized RNAs (28-30 nt) in the target gene region of the piRNA triggers. Sequence identities are indicated in the legend. DS, double-stranded triggers; SS, single-stranded triggers. 1U-10A reads, which are characteristic of bona fide ping-pong piRNAs show the greatest abundance. (FIG. 5D, FIG. 5E) Phasing signature plots separated by off-target and on-target strands for (FIG. 5D) single-stranded piRNA triggers and (FIG. 5E) double-stranded triggers. (FIG. 5F) Balloon plot showing reads with Dicer-2 nt overhangs for the DS and SS triggers. Color and size of circles scale with the abundance of 2-nt overhang pairs. Left shows the sequence identities of small RNAs analyzed (N=any residue, H=U/A/C, D=A/T/G, and W=A/T).

FIGS. 6A-6F show processing of piRNA triggers. (FIG. 6A, FIG. 6B, FIG. 6C, FIG. 6D) Characterization of the piRB-6 locus. (FIG. 6A) Phasing analysis of trailing 1U reads shows greater phasing signature on the plus strand of the locus compared to the antisense strand. (FIG. 6B) Overlap analysis for the piRB-6 locus showing a peak at 10-nt overlaps. (FIG. 6C) Enrichment of 22-nt reads that overlap by 2 nt at piRB-6. (FIG. 6D) Read accumulation at piRB-6. Alignments are colored by identity. Blue represents phasing piRNAs characterized by long 28-20-nt 1U reads that do not overlap by 10 with antisense reads and therefore unlikely to be involved in ping-pong. Red are ping-pong piRNAs being 28-30-nt reads that have 1U/10A sequences that also overlap by 10. Orange is siRNAs being 22-nt reads that have 2-nt overhangs with a 1U/A and 10A/U. The region cloned for the piRNA triggers indicated by dashed line box. The site of target sequence insertion is shown by the gray line. Y-axis shows read density. (FIG. 6E) Read accumulation using the color coding in part D at the sequence target sequence of piRNA triggers. Similarly, y-axis represents read density. Positive strand depicted at top of graphs is complementary to target. FIG. 6 (E, FIG. 6F) Quantification of read identities by strand for plots shown in FIG. 6E. (FIG. 6D, FIG. 6E) Color scheme same as used in (FIG. 6D, FIG. 6E). AG=AGLU1 and AQ=AQP1. FIG. 6 (G) Diagram showing the consequences of using different piRNA trigger configuration. Blue represents phasing strand of piRB-6 and red the complementary. (FIG. 6D) Same color scheme in (FIG. 6D) used to represent reads.

FIG. 7A-7E show characterization of the 50 most highly expressed small read biases and long read biased loci. (FIG. 7A) 50 most high expressing long read (25-30 nt) biased loci. Heat map shows distribution of reads between 16 and 40 nt. (FIG. 7B) 50 most high expressing short read (19-23 nt). si, siRNA sized; pi, piRNA sized biased-loci. Heat map shows distribution of reads between 16 and 40 nt Yellow shows density of all reads. (FIG. 7A, FIG. 7B, FIG. 7C) 3D scatterplot for the loci in (FIG. 7A, FIG. 7B) assessed by locus length, expression, and 1U bias. (FIG. 7D) 50 top long (25-31 nt) read loci where orange shows multi-mapping long reads and violet uniquely mapping long reads. (FIG. 7E) 50 top short (19-23 nt) read loci where orange shows multi-mapping short reads and violet uniquely mapping short reads.

FIG. 8. Visualization of RNA structure and small RNA expression at curated hairpin RNA loci. Top part of each panel depicts RNA structure with lines connecting one or more bases indicating pairing. Bottom panel is a density plot showing relative read depth across the locus. Red color indicates accumulation of 20-23-nt reads that map to more than one position in the genome. Blue indicates 20-23-nt reads that map uniquely to the locus. Yellow shows density of all read sizes.

FIG. 9A-9B. Proposed mechanism for piRNA/siRNA trigger. (FIG. 9A) Configuration of piRNA trigger where piRNA locus regions are modified to house the sequence of the target gene. (FIG. 9B) Single-stranded and dsRNA piRNA triggers developed in the work.

FIG. 10A-10D show evidence of piRNA-based RNAi in the potato psyllid. Nymphs were fed seven different synthetic RNAs targeted to v-ATPase-D. RNAs are shown in FIG. 10A. FIG. 10B shows sequencing of small RNAs after feeding of conventional dsRNA to animals. FIG. 10C shows efficacy of piRNA locus 1 RNas. FIG. 10D shows efficacy of piRNA locus 2 RNAs.

### DEFINITIONS

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “piwi-interacting RNA” or “piRNA” are used interchangeably and refer to a class of non-coding RNA molecules expressed in cells. piRNAs form RNA-protein complexes through interactions with piwi-subfamily Argonaute proteins. These piRNA complexes are involved various genomic modifications, including epigenetic and post-transcriptional silencing. These piRNA complexes are also involved in the regulation of genetic elements in germline cells.

As used herein, the terms “trigger” or “piRNA trigger” or “trigger piRNA” or “primary piRNA” or are used interchangeably to refer to the piRNA sequence that induces production of secondary piRNAs in a conducive environment. For example, a “piRNA trigger” may be a piRNA sequence that, when placed in an appropriate vector and delivered to a cell or organism, induces production of new piRNAs within the cell or organism. The new piRNAs may be produced by the “ping-pong” mechanism. Alternatively, or in addition, the new piRNAs may be produced by “piRNA phasing”. The new piRNAs are referred to herein as “secondary piRNA” or “responder piRNA”.

As used herein, the terms “small interfering RNA” or “siRNA” are used interchangeably to refer to a class of double-stranded non-coding RNA molecules. siRNA operates within the RNA interference (RNAi) pathway by interfering with expression of specific genes by degrading mRNA after transcription, thus preventing translation.

As used herein, the term “gene expression” or linguistic variants thereof refer to the process of converting genetic information encoded in a gene into RNA (e.g., mnRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refer to regulation that increases and/or enhances the production of gene expression products (e.g. RNA or proteins), while “down-regulation” or “repression” or “silencing” refer to regulation that decrease production. Molecules (e.g. transcription factors) that are involved in up-regulation or down-regulation are often called “activators” and “repressors,” respectively.

As used herein, the term “gene silencing” or “silencing” when used in reference to a gene or gene expression refers to methods for interrupting or suppressing expression of a gene. Gene silencing can occur at the transcriptional or translational level. Gene silencing can indicate a partial suppression of gene expression (e.g. a reduction of gene expression).

Alternatively, gene silencing can indicate a complete suppression of gene expression (e.g. an elimination of gene expression).

As used herein, the term “gene of interest” or “GOI” are used interchangeably herein to refer to the gene for which modulation of expression is intended. For example, the GOI may be the gene for which silencing of gene expression is desired.

As used herein, the term “hemipteran” refers to an order of insects that share a common arrangement of sucking mouthparts. The defining feature of hemipterans is their “beak” in which the modified mandibles and maxillae form a “stylet” which is sheathed within a modified labium. Hemiptera belong to the insect superorder Paraneoptera. Hemiptera includes multiple suborders, including auchenorrhyncha (e.g. cicadas, leafhoppers, treehoppers, planthoppers, froghoppers), coleorrhyncha (e.g. moss bugs), heteroptera (e.g. shield bugs, seeds bugs, assassin bugs, flower bugs, sweetpotato bugs, water bugs), and stemorrhyncha (e.g. aphids, whiteflies, scale insects).

The terms “RNA interference” or “RNAi” as used interchangeably herein broadly refer to biological processes wherein RNA molecules are involved in sequence-specific gene suppression.

The term “synthetic” when used in reference to nucleic acid molecules (e.g. RNA) refers to non-natural molecules made directly (e.g., in a laboratory) or indirectly (e.g., from expression in a cell of a construct made in a laboratory) by mankind.

As used herein, the term “transposon” or “transposable element” refers to a DNA sequence that can move and integrate into different locations within the genome. “Transposition” removes to the movement of a transposon. Transposition can create and/or reverse mutations within the genome.

### DETAILED DESCRIPTION OF THE DISCLOSURE

In some aspects, provided herein is a gene silencing approach. In some embodiments, provided herein are methods for gene silencing that exploits piwi-associated RNA (piRNA) biology. This cellular mechanism is a form of RNA interference where small RNA molecules specifically trigger the destruction of “on-target” genes. Multiple mechanisms produce piRNAs such as “ping-pong” and “phasing” mechanisms.

Generally speaking, the ping-pong mechanism of piRNA biogenesis involves piRNAs recognition of their complementary targets, thus causing the recruitment of piwi proteins. piRNAs associate with Piwi proteins with a high frequency of sequence complementarity over 10 nucleotides at their 5′ ends. This sequence complementarity is referred to as the “ping-pong signature”. Association of the piRNAs with piwi proteins results in cleavage of the transcript at a point ten nucleotides from the 5′ end of the primary piRNA, producing the secondary piRNA. These secondary piRNAs are often targeted toward sequences that possess an adenine at the tenth position. The ping pong cycle acts to disrupt gene expression at the transcriptional level.

The “phasing” mechanism of piRNA production involves the targeting and cleavage of a complementary target by a piwi protein associated with piRNA. Once cleaved, the targeted transcript is then processed further by additional enzymes (e.g. endonucleases), which leads to the loading of Piwi protein with sequential fragments of the targeted transcript. In this way, the piRNA sequence cleaves a complementary target that is then sliced at periodic intervals (e.g. intervals of approximately 27 nucleotides in length) that are sequentially loaded into Piwi protein. Once loaded with piRNA, Piwi then enters the germ cell nucleus to co-transcriptionally silence nascent complementary transcripts.

Although the ping-pong and phasing mechanisms are different, a unifying principle is that pre-existing piRNAs convert other cellular RNAs into new piRNAs. When this happens piRNAs bind through complementary base pairing to the target RNAs, which activates cleavage of the target and recruitment of the newly produced RNA fragments into the pathway. Cleavage patterns characteristic of piRNAs can be identified using analysis of high-throughput sequencing.

In some embodiments, methods described herein use existing piRNAs to drive gene silencing of target genes through the generation of on-target piRNAs. This may be performed by feeding animals synthetic RNAs that induce gene silencing via piRNA production. In some embodiments, the synthetic RNAs are designed by fusing the sequences of curated piRNA sequences to the target sequence. By formulating the curated sequence-specific piRNA modes (e.g., ping-pong, phasing) can be invoked. The result is a molecule that after ingestion triggers gene silencing in a mechanism distinct from classic double-stranded RNA (dsRNA) approaches. Such methods enable single-stranded RNA-based gene silencing.

The methods described herein offer an extraordinarily non-toxic approach to gene silencing that takes advantage of sequences specific to a particular animal species. This approach is appropriate for most arthropods, which include major agricultural pests and disease vectors. The methods described herein were tested in whitefly Bemisia tabaci where this piRNA-mediated gene silencing was found to be equally effective as the dsRNA-initiated methods that rely on the production of short-interfering RNA (siRNA). Moreover, the methods described herein were tested in a psyllid model (e.g. potato psyllid) where piRNA was found to outperform the dsRNA-mediated approach to gene silencing.

In some aspects, provided herein are synthetic RNAs. In some embodiments, provided herein is a synthetic RNA comprising a piRNA sequence, and a target sequence for a gene of interest. The synthetic RNAs described herein may be used to modulate expression of the gene of interest. In some embodiments, the synthetic RNAs described herein find use in methods to induce silencing of the gene of interest. Silencing of the gene of interest may occur by one or more mechanisms, including ping-pong biogenesis of secondary piRNAs and/or production of secondary piRNAs through piRNA phasing.

For the synthetic RNAs described herein, both the “sense” and “antisense” strands are expressly contemplated. Accordingly, for any sequence described herein both the sense sequence itself and the complementary (e.g. antisense) sequence are expressly contemplated.

The synthetic RNA may be single stranded or double stranded. In some embodiments, the synthetic RNA is single stranded. In some embodiments, the synthetic RNA is double stranded.

In some embodiments, the target sequence for the gene of interest is sandwiched between two components of the piRNA sequence. For example, in some embodiments the piRNA sequence comprises a left flanking sequence and a right flanking sequence, and the target sequence for the gene of interest is sandwiched in between the left flanking sequence and the right flanking sequence.

Any suitable gene of interest may be the target of the synthetic RNAs described herein. The gene of interest may be selected based upon the desired result of gene silencing. For example, the gene of interest may be selected to generate a lethal result for the organism when the gene of interest is silenced. As another example, the gene of interest may be selected to create infertile organisms. For example, the gene of interest may be selected such that the male and/or female organism becomes infertile if the gene of interest is silenced. In some embodiments, the gene of interest is a gene provided in Table 1. In some embodiments, the gene of interest is a gene in a hemipteran organism. In some embodiments, the gene of interest is the gene of interest is aquaporin (AQP1), alpha glucosidase 1 (AGLU1), v-ATPase-A, v-ATPase-B, v-ATPase-D, v-ATPase-E, Delta-24 sterol reductase (D-24), cholesterol desaturase (C7), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP-family verprolin-homologous protein (WAVE), or Actin related 2/3. The target sequence may be present in the gene of interest. In some embodiments, the target sequence may be complementary to a corresponding region within the gene of interest.

In some embodiments, the target sequence for the gene of interest may be a region corresponding to 20 or more contiguous nucleotides present in the gene of interest. In some embodiments, the target sequence for the gene of interest may comprise 20 or more contiguous nucleotides complementary to or encoding an amino acid sequence that is complementary to a corresponding region present in the gene of interest.

The target sequence for the gene of interest may comprise 20 or more contiguous nucleotides present in the gene of interest or encoding a corresponding amino acid sequence present in the gene of interest. For example, the target sequence may comprise at least 20, at 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous nucleotides present in the nucleotide sequence encoding the gene of interest or encoding a corresponding amino acid sequence present in the gene of interest.

In some embodiments, the target sequence for the gene of interest may comprise 20 or more contiguous nucleotides complementary to or encoding a corresponding amino acid sequence that is complementary to a corresponding region within the gene of interest. For example, the target sequence may comprise at least 20, at 30, at least 40, at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, at least 250, at least 260, at least 270, at least 280, at least 290, or at least 300 contiguous nucleotides complementary to or encoding a corresponding amino acid sequence that is complementary to a corresponding region within the gene of interest.

In some embodiments, the gene of interest is aquaporin 1 (AQP1). The amino acid sequence AQP1 from Bemisia tabaci is:

(SEQ ID NO: 1)
MEDISSSGEE ISMKAISKVI GVPDIRDGPT LTKCIVAEFV
GTLLLVLIGC MSVAFVHQDN FVDVVKIAMA FGLIIASMVQ
AIGHVSGCHI NPAVTCGLAV SGHVSIIKGM LYIVAQCLGA
ICGAIILNEI TPKTGYTAAG NLGVTTLSTG VSDLQGVAIE
ALITFVLLLV VQSVCDGKRT DIKGSIGVAI GFAIACCHLA
AIKYTGASMN PARSLGPAFV SGIWDKHWVY WAGPILGGVT
ASLLYAITFK AKKRSDESSY DF

In some embodiments, the target sequence comprises at least 20 contiguous nucleotides encoding a fragment of SEQ ID NO: 1. In some embodiments, the target sequence comprises at least 20 contiguous nucleotides encoding a fragment complementary to a corresponding region within SEQ ID NO: 1. In some embodiments, the target sequence may comprise at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in SEQ ID NO: 2. In some embodiments, the target sequence comprises at least 20 contiguous nucleotides present in SEQ ID NO: 2. In some embodiments, the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to the same number of contiguous oligonucleotides present in SEQ ID NO: 2. In some embodiments the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides present in SEQ ID NO: 2. In some embodiments, the target sequence comprises the nucleotide sequence of SEQ ID NO: 2.

In some embodiments, the gene of interest is alpha glucosidase 1 (AGLU1). The amino acid sequence of AGLU1 from Bemisia tabaci is:

(SEQ ID NO: 68)
WRDQELLSPDLDPTNYWNYNRSRTMDQPNTYRLITRFREVFDFYTKKEG
KTKVLMTEAYTTLDRTMDYYQFEGKPGAHMPFNFFFITHVSGRSPAKDY
QKAIQ.

In some embodiments, the target sequence comprises at least 20 contiguous nucleotides present in the nucleotide sequence encoding AGLU1 (e.g. encoding the amino acid sequence of SEQ ID NO: 68). In some embodiments, the target sequence comprises at least 20 contiguous nucleotides encoding a fragment complementary to a corresponding region within AGLU1 (e.g. within SEQ ID NO: 68). In some embodiments, the target sequence comprises at last 20 contiguous nucleotides encoding a corresponding amino acid sequence present in AGLU1 (e.g. present in SEQ ID NO: 68). In some embodiments, the target sequence comprises at least 20 contiguous nucleotides encoding a complementary amino acid sequence within AGLU1 (e.g. present in SEQ ID NO: 68). In some embodiments, the target sequence comprises at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in SEQ ID NO: 3. In some embodiments, the target sequence comprises at least 20 contiguous nucleotides present in SEQ ID NO: 3. In some embodiments, the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to the same number of contiguous nucleotides present in SEQ ID NO: 3. In some embodiments the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides present in SEQ ID NO: 3. In some embodiments, the target sequence comprises the nucleotide sequence of SEQ ID NO: 3.

In some embodiments, the gene of interest is v-ATPase-D. The nucleotide sequence of v-ATPase-D in the potato psyllid is SEQ ID NO: 16 and in the Asian citrus psyllid is SEQ ID NO: 33. In some embodiments, the target sequence comprises at least 20 contiguous nucleotides complementary to a corresponding region within SEQ ID NO: 16 or SEQ ID NO: 33. In some embodiments, the target sequence may comprise at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in SEQ ID NO: 16 or SEQ ID NO: 33. In some embodiments, the target sequence comprises at least 20 contiguous nucleotides present in SEQ ID NO: 16 or SEQ ID NO: 33. In some embodiments, the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to the same number of contiguous oligonucleotides present in SEQ ID NO: 16 or SEQ ID NO: 33. In some embodiments the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides present in SEQ ID NO: 16 or SEQ ID NO: 33. In some embodiments, the target sequence comprises the nucleotide sequence of SEQ ID NO: 16 or SEQ ID NO: 33.

Another suitable target sequence for v-ATPase-D is gagaaggcagcttctttcatgacttcacccatgagggtttttgtctcgatgattttgctcaggatcatacggaatctcatctggagagcatc agcttcttcttaagcaaactgtgtcccttctgagcccccttgagacgggacttcatga (SEQ ID NO: 70). In some embodiments, the target sequence comprises at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in SEQ ID NO: 70. In some embodiments, the target sequence comprises a nucleic acid sequence having at least 20 contiguous nucleotides present in SEQ ID NO: 70. In some embodiments, the target sequence comprises at least 20, at least 30, at least 40, at least 50, at least 60, at least 70, at least 80, at least 90, or at least 100 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to the same number of contiguous nucleotides present in SEQ ID NO: 70. In some embodiments, the target sequence comprises the nucleotide sequence of SEQ ID NO: 70.

In some embodiments, the target sequence is sandwiched between a left flanking region and the right flanking region of a piRNA sequence. In some embodiments, the piRNA sequence (e.g. the left flanking region and the right flanking region) is selected to stimulate primarily production of secondary piRNAs. In some embodiments, the left flanking region of the piRNA sequence comprises the nucleotide sequence AGCAGCTTCTTGCCTCTGATTCCACGGTTTCTTCTTAAAGGGCCCCGACGACTGCT GCGGGCCTTGATAAGGCGCGCTCCTGTTATTTGCCTCACGGAACGTCTTTTCCGC GGCCATCATTGCGTCCATTGATCGGATCAAATCTTGCCTCATTGCATCCACGGCT CGAGTATTCCTATCCGTATCCGCACGATTTAGATCAACTGCGTGTACCAAAGTCG CTAGGGCGTTCTCATTGGCCTTCACCCGGGATTCTAAGGATGATTCCTGCCCCGT ATAGTGATTTACGGCCAAAATAGCGCCCCTTCCTTTGCTGGTCGCGGCTACTGCT AGCTTCGCATT (SEQ ID NO: 4). In some embodiments, the left flanking region of the piRNA sequence comprises a nucleotide sequence having at least 50% sequence identity with SEQ ID NO: 4. For example, the left flanking region of the piRNA sequence may comprise a nucleotide sequence having at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with SEQ ID NO: 4.

In some embodiments, the right flanking region of the piRNA sequence comprises the nucleotide sequence TCCGTCGAGTTAACTTTAGCCAAGCCCGCTAGTTTTCTCTTCGCTTGAACGTAATC CAACGGGTCCTCATTTTCTCCCTGCGTTCGCGCCGAGAATTTCGTGAGGGCATCC TCGTCGCTGTCAAAGTATTGGATCAATTTCTTCTTTACTTCCTCAAAAGTCCTGCA GTTACCGAACGCTACCTCTTCATTGTCGTAGTACTGGATGGCACGTTTCGCTAAG TGATTTCTGAGTTGGTCCCGCTTTTCTTGATCCGAACATTTCTTATAGAAATTTTC AAAATCTTTTAGAAATTCTCTAACGTCGTAGTCAGCTTCTCCTTTGAATAGTTTTC TAAACGGCGGTGCCTTAATCGTCACCGTAGGT (SEQ ID NO: 5). In some embodiments, the right flanking region of the piRNA sequence comprises a nucleotide sequence having at least 50% sequence identity with SEQ ID NO: 5. For example, the left flanking region of the piRNA sequence may comprise a nucleotide sequence having at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with SEQ ID NO: 5.

In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 4 and the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 5. In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 4, the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 5, and the target sequence comprises at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in SEQ ID NO: 2 or SEQ ID NO: 3.

In some embodiments, the piRNA sequence (e.g. the left flanking region and the right flanking region) is selected to stimulate production of secondary piRNAs and siRNA. Such a piRNA sequence is referred to herein as a “no bias” sequence. In some embodiments, the left flanking region comprises the nucleotide sequence: TTGCGTTCCTGCTCCCTTTGCCCTTTACCGCGCTCAATTATCTCTATTAGAA CCGGAGATATTCGGTTTACAAAAATTTTTTGGGGCCCAGCCCCCCTTAATCCTTTC CCTATGGACTTCCTATATGGCCCCAGAGGTAGCCCCCGGGGGTTAGGCAAATAAT CCCAAAAAATTCCCAAATTCTAACGGAAATGTGGCACTACCGCCCCTACGTCACT CTGGCTATGACGTAGTTGAT (SEQ ID NO: 6) In some embodiments, the left flanking region of the piRNA sequence comprises a nucleotide sequence having at least 50% sequence identity with SEQ ID NO: 6. For example, the left flanking region of the piRNA sequence may comprise a nucleotide sequence having at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with SEQ ID NO: 6.

In some embodiments, the right flanking region comprises the nucleotide sequence TTACGTGCCGTTACACCGGTTACCGACATCAGGTTCCTTCAAATCGGACACGGGC GCCCCTCCCCGAGGGGATGCCAATGGGGGGAGGTCCCAGGCCGAAGCCTGACTT TCTACTACCTCCGGAGCTGTGCCCTTCTCTGCACGTCCCAGTTGAGCACTGGTGG GCTGACCTCGGGGACAAGGTCGCCTTAACTTACCG (SEQ ID NO: 7). In some embodiments, the right flanking region of the piRNA sequence comprises a nucleotide sequence having at least 50% sequence identity with SEQ ID NO: 7. For example, the right flanking region of the piRNA sequence may comprise a nucleotide sequence having at least 50%, at least 60%, at least 70%, at least 80%, at least 90%, at least 91%, at least 92%, at least 93%, at least 94%, at least 95%, at least 96%, at least 97%, at least 98%, at least 99%, or 100% sequence identity with SEQ ID NO: 7.

In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 6 and the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 7. In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 6, the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 7, and the target sequence comprises at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in SEQ ID NO: 2 or SEQ ID NO: 3.

In some embodiments, the synthetic RNA comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) with SEQ ID NO: 8. In some embodiments, the synthetic RNA comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) with SEQ ID NO: 9. Such synthetic RNA sequences may find use in methods for silencing expression of AQP1.

In some embodiments, the synthetic RNA comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) with SEQ ID NO: 10. In some embodiments, the synthetic RNA comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) with SEQ ID NO: 11. Such synthetic RNA sequences may find use in methods for silencing expression of AGLU1.

In some embodiments, the left flanking region of the piRNA sequence comprises the nucleotide sequence

(SEQ ID NO: 71)
AATCTTTCAAATTACCACTAAACTCTTTCAGCTTCAATATTGGAAGTTT
GCACTGATACTGAGCTATATTACTTCCATTAGCTGATACAAAACTACCA
TTTTGATCATCTGGTGTATGTACTGTCTGACTACTCTGAATCTTACTCT
CAACCAGAT

In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 71.

In some embodiments, the right flanking region of the piRNA sequence comprises the nucleotide sequence

(SEQ ID NO: 72)
AATCTTTCAAATTACCACTAAACTCTTTCAGCTTCAATATTGGAAGTTT
GCACTGATACTGAGCTATATTACTTCCATTAGCTGATACAAAACTACCA
TTTTGATCATCTGGTGTATGTACTGTCTGACTACTCTGAATCTTACTCT
CAACCAGAT

In some embodiments, the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 72.

In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 71 and the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 72.

In some embodiments, the left flanking region of the piRNA sequence comprises the nucleotide sequence TTTCTTCTGAATGTTGAAATGATGTTGATGGAATTGGATTTGACATAGTATCTTCT GGGCTTGCCTGTATTAGTGGTGTTTTTTGTGCTTCTTTATAGGGCTTTCTTTTCTTG GCAGGGGGTTGTAAGTAAGAGGGAAAAGCTGTAAAAACTGAAGGAA (SEQ ID NO: 73). In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 73. In some embodiments, the right flanking region of the piRNA sequence comprises the nucleotide sequence TTTCTTCTGAATGTTGAAATGATGTTGATGGAATTGGATTTGACATAGTATCTTCT GGGCTTGCCTGTATTAGTGGTGTTTTTTGTGCTTCTTTATAGGGCTTTCTTTTCTTG GCAGGGGGTTGTAAGTAAGAGGGAAAAGCTGTAAAAACTGAAGGAA (SEQ ID NO: 74). In some embodiments, the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 74. In some embodiments, the left flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 73 and the right flanking region of the piRNA sequence comprises a nucleotide sequence having at least 50% (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) sequence identity with SEQ ID NO: 74.

In some embodiments, the synthetic RNA comprises the nucleotide sequence AATCTTTCAAATTACCACTAAACTCTTTCAGCTTCAATATTGGAAGTTTGCACTGA TACTGAGCTATATTACTTCCATTAGCTGATACAAAACTACCATTTTGATCATCTGG TGTATGTACTGTCTGACTACTCTGAATCTTACTCTCAACCAGATgagaaggcagcttctttcat gacttcacccatgagggtttttgtctcgatgattttgctcaggatcatacggaatctcatctggagagcatcagctttcttcttaagcaaact gtgtcccttctgagcccccttgagacgggacttcatgaAATCTTTCAAATTACCACTAAACTCTTTCAGC TTCAATATTGGAAGTTTGCACTGATACTGAGCTATATTACTTCCATTAGCTGATAC AAAACTACCATTTTGATCATCTGGTGTATGTACTGTCTGACTACTCTGAATCTTAC TCTCAACCAGAT (SEQ ID NO: 75). In some embodiments, the synthetic RNA comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) with SEQ ID NO: 75. Such a synthetic RNA sequence may find use in methods for silencing expression of v-ATPase-D.

In some embodiments, the synthetic RNA comprises the nucleotide sequence TTTCTTCTGAATGTTGAAATGATGTTGATGGAATTGGATTTGACATAGTATCTTCT GGGCTTGCCTGTATTAGTGGTGTTTTTTGTGCTTCTTTATAGGGCTTTCTTTTCTTG GCAGGGGGTTGTAAGTAAGAGGGAAAAGCTGTAAAAACTGAAGGAAgagaaggcagc ttctttcatgacttcacccatgagggtttttgtctcgatgattttgctcaggatcatacggaatctcatctggagagcatcagcettcttcttaa gcaaactgtgtcccttctgagcccccttgagacgggacttcatgaTTTCTTCTGAATGTTGAAATGATGTTGA TGGAATTGGATTTGACATAGTATCTTCTGGGCTTGCCTGTATTAGTGGTGTTTTTT GTGCTTCTTTATAGGGCTTTCTTTTCTTGGCAGGGGGTTGTAAGTAAGAGGGAAA AGCTGTAAAAACTGAAGGAA (SEQ ID NO: 76). In some embodiments, the synthetic RNA comprises a nucleotide sequence having at least 50% sequence identity (e.g. at least 50%, at least 60%, at least 70%, at least 80%, at least 90%, at least 91%, at least 92%, at least 93%, at least 94%, at least 95%, at least 96%, at least 97%, at least 98%, at least 99%, or 100%) with SEQ ID NO: 76. Such a synthetic RNA sequence may find use in methods for silencing expression of v-ATPase-D.

In some embodiments, the target gene is found in a psyllid. For example, the target gene may be a gene in the potato psyllid (PoP) or the Asian citrus psyllid (ACP). Suitable target genes that may be particularly well suited for gene editing in psyllids are shown in Table 1.

TABLE 1
Potential potato psyllid (PoP) and
Asian citrus psyllid (ACP) genes:

| | |
|---|---|
| GROUP 1 | v-ATPase-A (7) |
| | v-ATPase-B (8) |
| | v-ATPase-D (9) |
| | v-ATPase-E (11) |
| GROUP 2 | D-24 (115) |
| | C7 (116) |
| | Crc (165 |
| GROUP 3 | Chitinase 7 (178) |
| | Chitin Synthase (181) |
| | Endochitinase (184) |
| GROUP 4 | Cortactin (96) |
| | Actin (30) |
| | WASP (93) |
| GROUP 5 | RAC1 (160) |
| | IRSp53 a.k.a. BAI (192) |
| | WAVE (190) |
| | Actin related 2/3 (34) |

In some embodiments, the target sequence may comprise at least 20 contiguous nucleotides encoding an amino acid sequence found in one of the above-listed target genes (e.g. the genes in Table 1). In some embodiments, the target sequence may comprise at least 20 contiguous nucleotides present in the nucleotide sequence encoding the target gene. The siRNA may comprise any suitable left and right flanking region sandwiching the target nucleotide sequence.

The sequences of suitable genes of interest, including those listed in Table 1, are provided below. In some embodiments, the target sequence comprises at least 20 contiguous present in any one of SEQ ID NO: 14-47. In some embodiments, the target sequence comprises at least 20 contiguous nucleotides complementary to a corresponding region within any one of SEQ ID NO: 14-47. In some embodiments, the target sequence may comprise at least 20 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to at least 20 contiguous nucleotides present in any one of SEQ ID NO: 14-47. In some embodiments, the target sequence comprises at least 20 contiguous nucleotides present in any one of SEQ ID NO: 14-47. In some embodiments, the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides having at least 80% sequence identity (e.g. at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity) to the same number of contiguous oligonucleotides present in any one of SEQ ID NO: 14-47. In some embodiments the target sequence comprises at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 contiguous nucleotides present in any one of SEQ ID NO: 14-47. In some embodiments, the target sequence comprises the nucleotide sequence of any one of SEQ ID NO: 14-47.

Sequences: Potato psyllid (ACP), Bactericera cockerelli
#P-7 BcAN_00119 v-ATPase A
ATGTCAACAGCATTAGGAAAAATGGTGGATGAAGATAGAGAAGGTAGATTTGGG
TTTGTGTATGCAGTATCAGGTCCTGTGGTAACAGCAGAGAAAATGTCAGGGTCTG
CTATGTACGAGCTGGTTCGAGTAGGTTACTTTGAACTGGTGGGTGAAATCATCCG
GCTGGAGGGTGATATGGCCACCATTCAAGTGTATGAAGAAACATCGGGTGTAAC
TGTTGGTGACCCTGTGTTGAGGACAGGCAAACCCTTATCTGTAGAGCTTGGTCCT
GGTATCCTGGGTAGTATTTTTGATGGTATCCAACGTCCACTGAAGGACATTTGTG
AGTTGTCTCAGAGCATTTACATCCCAAAAGGAGTTAACATTCCTGCCTTGAACAG
AGATGTTAGCTGGGAGTTCAATCCAATGAACTTAAAGATTGGTAGTCACATGACC
GGTGGAGATCAGTATGGTATTGTACATGAGAATACACTTGTTAAACATAAAATG
ATCATGCCACCCAAAGCAAAGGGAACTGTAACATACATTGCTCCAGCTGGTAATT
ACAAGGTAGATGAAGTTGTTATTGAAACTGAATTTGATGGAGAGAAGAGTAAAT
ACACTATGGTTCAAGTATGGCCTGTACGTCAACCTCGCCCTGTCACCGAAAAACT
CCCTGCAAATTACCCCCTTCTAACAGGTCAACGAGTCCTTGATTCTCTGTTTCCTT
GTGTTCTTGGAGGAACAACTGCCATTCCTGGTGCCTTTGGCTGTGGTAAAACTGT
CATCTCACAAGCTTTATCTAAGTATTCCAATTCTGATGTGATTGTCTATGTAGGAT
GTGGTGAACGAGGTAATGAAATGGCAGAAGTATTGAGAGATTTCCCTGAACTTT
CAATTGAGGTGGATGGAGTCACAGAATCCATCATGAAACGTACCACCTTGGTAG
CCAACACATCAAACATGCCTGTAGCTGCTCGAGAAGCTTCTATTTACACTGGTAT
CACACTGTCTGAGTACTTCAGGGACATGGGTTACAATGTGTCTATGATGGCTGAC
TCCACGTCACGATGGGCTGAGGCTTTGAGAGAAATCTCAGGACGTCTAGCTGAG
ATGCCTGCTGACAGTGGGTATCCTGCTTACCTGGGTGCAAGACTTGCCTCCTTCT
ATGAGCGTGCTGGTAGAGTTAAGTGTTTGGGTAACCCAGACAGAGAGGGTTCTG
TTAGTATTGTGGGTGCTGTGTCTCCCCCTGGTGGTGATTTCTCAGACCCTGTCACT
TCAGCAACATTGGGTATTGTTCAAGTGTTCTGGGGTCTTGACAAAAAACTTGCTC
AGAGGAAACATTTTCCATCCATCAACTGGCTCATTTCCTACAGTAAGTACATGAG
AGCCCTGGATGATTTCTATGAAAAAAACCACCCTGAGTTTGTACCCCTCAGAACA
AAAGTAAAAGAAATTCTTCAAGAAGAAGAAGATTTGTCAGAAATTGTGCAACTG
GTTGGTAAGGCATCCCTGGCTGAATCTGATAAGATCACTTTGGAAGTTGCCAAAC
TGCTGAAAGATGATTTCCTCCAACAAAACAGTTACTCCCCCTATGACAGGTTCTG
TCCCTTCTACAAAACTGTTGGTATGTTGCGTAATACTATTGCTTTCTATGATATGT
CCCGTCATGCAGTTGAGTCTACTGCCCAATCAGAAAACAAGATCACATGGTCTGT
GATAAGGGACAGTATGAACAACATCTTGTATCAACTTTCGTCCATGAAATTCAAA
GACCCAGTCAAGGATGGTGAAGCTAAAATAAGAGCAGACTTTGATCAACTCTAT
GAAGACATTCAGCAAGCATTCCGTAACTTAGAAGACTAA (SEQ ID NO: 14)
#P-8 BcAN_02379 v-ATPase B
ATGTCGATATCTACCAAGCAGGCTCTACGGGAAAACGTCCTGGCTGTCACGCGG
GATTACATTTCGCAGCCGAGAATCACATACAAAACTGTGTCTGGTGTCAATGGAC
CCCTGGTCATCTTGGATGAAGTGAAGTTCCCTAAGTATGCTGAAATTGTACAGCT
CCGTTTGAACGATGGATCTTACCGTGCTGGTCAAGTTCTGGAAGTCAGTGGCTCT
AAAGCTGTGGTCCAGGTATTTGAAGGTACCTCTGGAATTGATGCGAAGAACACT
GTCTGTGAGTTCACGGGGGATATCTTGAGAACACCAGTGTCTGAAGACATGTTGG
GGCGTGTGTTCAACGGAAGTGGAAAGCCTATTGACAAAGGACCCCCTATCCTAG
CTGAAGACTACCTTGACATTGAAGGTCAACCCATCAACCCCTACTCAAGAACCTA
CCCACAGGAAATGATTCAGACTGGTATCTCCGCTATTGATGTGATGAACTCCATT
GCCCGAGGACAGAAGATTCCCATCTTCTCCGCTGCTGGTCTACCCCACAATGAAA
TCGCCGCCCAGATCTGTCGACAAGCTGGCCTGGTCAAGATGCCGGGCAAGTCAG
TTCTGGATGACTCGGAAGATAACTTCGCCATTGTGTTTGCTGCCATGGGAGTCAA
CATGGAGACTGCCCGGTTCTTCAAACAAGACTTCGAGGAAAACGGGTCCATGGA
GAACGTGTGTCTCTTCTTGAACTTGGCCAACGACCCGACCATAGAGCGGATCATC
ACTCCCCGCCTCGCCCTGACCACCGCCGAGTTCCTCGCCTACCAGTGCGAGAAGC
ATGTGCTCGTCATTCTCACCGACATGTCCTCCTACGCCGAGGCTCTGCGTGAGGT
GTCAGCTGCCCGGGAGGAAGTCCCCGGGAGACGTGGGTTCCCCGGTTACATGTA
CACCAACTTGGCTACTATCTACGAGAGGGCCGGACGTGTGGAGGGCAGGAACGG
GTCCATCACTCAGATTCCCATCCTGACCATGCCCAACGACGACATCACCCATCCC
ATCCCTGACTTGACCGGCTACATCACCGAGGGCCAGATCTACGTGGACAGACAG
CTCCACAACAGACAGATCTACCCCCCTATCAATGTGCTGCCCTCCCTGTCCCGTC
TCATGAAGTCTGCCATCGGCGAGGACATGACACGCAAGGATCACTCCGACGTTTC
CAACCAGTTGTACGCCTGCTACGCCATAGGCAAGGACGTGCAAGCCATGAAGGC
GGTGGTGGGAGAGGAAGCTCTGACACCCGACGATCTGCTGTATCTGGAGTTCCTC
ACCAAGTTCGAGAAGAACTTTGTGAGCCAAGGGAACTACGAGAACCGCACCGTG
TACGAGAGTCTGGACATCGGCTGGCAGCTCCTGCGTATCTTCCCCAAGGAGATGC
TGAAGAGAATCCCTGCCGCCACGCTGGCCGAGTTCTACCCGCGTGACTCCAGAC
ACACTGGCGCCAAGTTAACACGCGCGCTGGTCACCCCTCCACCCCCCTACCATTG
TCTAGACAAGTGGGAACCTTACAGCCCCTCTACCCCCCCTACCATTGTGGTCTAG
(SEQ ID NO: 15)
#P-9 BcAN_10854 v-ATPase D
ATGTCAGGTAAAGATAGACTTGCCATTTTTCCGTCCCGAGGAGCTCAGTCCCTCA
TGAAGTCCCGTCTCAAGGGGGCTCAGAAGGGACACAGTTTGCTTAAGAAGAAAG
CTGATGCTCTCCAGATGAGATTCCGTATGATCCTGAGCAAAATCATCGAGACAAA
AACCCTCATGGGTGAAGTCATGAAAGAAGCTGCCTTCTCCTTAGCAGAGGCGAA
ATTCACAACAGGGGATTTCAATCAGGTAGTCCTACAAAATGTAACCAAGGCACA
AATCAAAATACGCACTAAGAAAGACAATGTTGCGGGTGTTACTCTTCCAGTGTTT
GAGAGTTACCAGGATGGTACGGATACCTACGAGCTAGCTGGTCTTGCCAGAGGA
GGTCAACAGCTCGCAAAGCTGAAGAAAAACTATCAGACAGCCATCAAACTCCTT
GTTGAGCTAGCCTCTCTACAAACATCCTTTGTAACCCTAGATGATGTTATTAAAA
TTACCAACCGCAGAGTGAACGCTATTGAGCATGTCATCATTCCTCGTATTGAAAG
AACTCTGGCGTATATTATTTCTGAGCTGGATGAACTAGAGAGAGAAGAGTTTTAC
CGTTTGAAGAAGATCCAGGATAAGAAGAAGGTGATCAAGGCAGCATCAGAAGC
ATTCAGAAAGTCCCGTAAATATGATGAGGAGCAAGCAATGAATATGCTTGATGA
AGGTGATGAAGACATTCTGTTCTAA (SEQ ID NO: 16)
#P-11 BcAN_02354 v-ATPase E
TTAATCATCAAATTTACGGTTAGGGTTACGACCAAATATAGCAACTCTAATGTCA
GGGATGATCTGTTGTGCAATCAATTCCAGACGAGCCTCCAAGGTATTGCTGATCT
TGATTTTTCCTCTTTGAGCCAAGAGCTCAATACCTCCTGTGGTATTAACAGGTAA
GAATTGTTCACTGTCAAGTTTCAAGTTAACTTCCTTACCAGCAACATCTTGGTATT
GCTTGGCCACCGTAGGTAAGACACCATTGACAAGTTCTTTGTCAGCTTCCCGGGC
ACGGATCAGAACATTGGGCTCCAATAATTGCAGCAGGCCTTGAACCATTAACTTT
TCAATGAGTTGGGTGTACTTGGGGCGGTCCTTGGACACCTCACCCAGTCTGTTCC
TGGCCTCTTCCAACACATTTCTGACATGATCTTCCCTCACCTTCAAAGCCTTCAGT
CGGGCCTGATTCAGCATGTTAGAGGACTGAATCTTCTTCTGCAGCTCAACTTGCT
TTTCTTTCCTGTCATAGTATTCCATAATCTTGAGACGCTGGTGTTGGACCAAACGC
CCTTTTTCGATGTTGAATTCTTCTTCTGCCTTAGCATCGATTTCCTCAGCCTTCTCG
TTGGCTTCTTGCTCGATGAAAGCCATCATGTGATTGATTTGCTTTTGAACGTCCGC
ATCGCTTAAAGCCAT (SEQ ID NO: 17)
#P-30 BcAN_01478 Actin
ATGTGTGACGACGATGTAGCCGCTTTGGTCGTGGACAATGGTTCCGGTATGTGCA
AGGCCGGATTCGCCGGTGATGACGCCCCCAGAGCCGTGTTCCCCTCAATCGTCGG
TAGACCCCGTCATCAGGGTGTCATGGTGGGTATGGGTCAAAAAGACTCCTACGTC
GGTGATGAGGCTCAGTCCAAGAGAGGTATCCTCACCCTGAAATACCCCATCGAG
CACGGTATCATCACCAACTGGGACGACATGGAGAAGATCTGGCATCACACCTTCT
ACAACGAGCTGAGAGTCGCCCCCGAGGAGCACCCCATCCTGCTGACGGAGGCAC
CCCTCAACCCCAAAGCCAACAGAGAGAAGATGACCCAGATCATGTTTGAGACGT
TCAACACCCCCGCCATGTACGTCGCCATCCAGGCTGTGCTCTCCCTGTACGCCTC
CGGTCGTACCACCGGTATCGTGCTCGACTCTGGAGATGGTGTCTCCCACACCGTC
CCCATCTATGAAGGTTACGCCCTCCCCCACGCCATCCTCCGTCTGGATCTGGCTG
GTCGTGACTTGACCGACTACCTGATGAAGATCCTCACCGAGAGAGGTTACTCCTT
CACCACCACCGCTGAGCGGGAAATCGTCCGTGACATCAAGGAGAAGCTCTGCTA
CGTCGCCCTGGACTTCGAGCAGGAGATGGCCACCGCCGCCGCCTCCACCTCCCTG
GAGAAGAGCTACGAGCTGCCCGACGGACAAGTCATCACCATCGGAAACGAGAG
ATTCCGTTGTCCCGAGGCTCTGTTCCAGCCTTCCTTCCTGGGTATGGAGTCTTGCG
GTATCCACGAGACCGTGTACAACTCCATCATGAAGTGCGATGTCGACATCAGAaa
ggacctgtacgccaacactgtcctgtccggtggtaccaccatgtaccccggtatcgccgacagaatgcagaaggaaatcactgccct
ggctccttccaccatcaagatcaagatcatcgctccccccgagagaaagtactccgtatggatcggtggttccatcctggcctctctgtc
caccttccagcagatgtggatctccaaacaggaGTACGACGAGTCCGGTCCCGGAATCGTCCACCGC
AAATGTTTCTAA (SEQ ID NO: 18)
#P-34 BcGS_01438 Probable actin-related protein 2/3
CTCAACTCGGGTTATAGTGCGCTGTAATTTAATGAAACTCCAACTTGAGGGATGG
TCTTGCTTATCTTAACTTTAATCCTATTTCATAAAAAAAGTTCTCGGTCCGAGGAG
GCTATACAACTGGTCTACACAATCACCTTATGAAGCTCTTTGTTgttgtaaccagcgtggcta
cagctgggggagtggtttgctttcactttgcttccgaaatacttggtctggaccaaagttattactgcagtggccgatgtgctatcttgaatt
cagggcagagttgttctgaacatggcagtgacttcatcatccgagagcttcgagaacaatcattctctcctgtggaacgttctccccgtg
attgtcttCTTCTCCGTGTTCTTCACTTCAGGCCGAGCTCTGTTCAACACCTTGAGGAA
GTCCGAGGTCTTCGCCCGCATGCGGGAGTGAATGTAGGCCTTGGAACATTTGATG
TGGTAGTGCAGATAGTCCCGGAACATGTGGATCAGATTGATGGTGTTCTCTCGGG
CCACCCGGTTTGTGTGTCTGGGAAACAGCACAAACGTAATGTAGCCAATGTTGTC
CCCTTGTCGGGCGTCCGTGTCTCTCAGCTCCAGGGGCGGCTCCTTGTGACTGAAC
AGCACCTGGGGCGCCGTGTGACTCGCCCGCCGCCCCTCCTTGAACTCCTGCATGA
ACACCTTGCCTATGATGACGTCATCCTCACCCCGGAATACTGTGCTGAACACCAC
CGTCACGCGGTCTGCCTTCGCCTCCACATACATAGTTTCTTCATTCCTATAGTTGA
TCACGGCTCTACTCTGACCTTCCTCTCCTCTTTCTTGAAAGTCAAAGTATTTTTCA
AACACAGATGCAAAACAATTCCTCTTTAAGAGACCAATTTTTTTGACTAGTTGCT
CCCAGTCAGAAGGAATATTTTCTAGGTCTATCAGTACAGATGCATTGTAGCCTGG
TTCAGGATCAGTCAACAGAGCACCATACTCTCGCCTCAACAGTTCATCAGCACCA
TGTTGCTCAAGCTCTTTGTAGAACTTGAGAGATATACTAGTCCTTATTTTAGTTTT
ATCACCACTAAGGTTGGAAATGTGAAATAATACTCCATCAAAATCTGCTATTGTG
ATATCTACTGATTCTGGTTTTGATCCAGCGATGGCATTTTTGAATTTCAAAGAAA
GAGTTTCCTCCACGATCCTGTTGTGTATTTCCAAGAGAATCATGTTGAAAATTAA
AATATTACTACAATTCCATAAAAT (SEQ ID NO: 19)
#P-93 BcGS_17389 Wiskott-Aldrich syndrome protein (WASP)
ATGAAACCTAACGCAACACCTCCTGAACAGAAAACGTCAAGTTTGTTGACACGA
GATGAAAATGAAGCTGTGTTCAAGCTTTTGGGTAATAGATGTCAGGCGCTTTCCA
CAACTGTGATACAGCTGTTCACCACAGACGGCCCTAACGATAACGAGTGGCACA
AGAGGTGTTTCGGCATTCTGTGCCTTGTCAAGGATAATCCCCGCAAATCCTATTT
CTTCCGACTCTACTGTTTAACGAGGAGACAACTGGTTTGGGAACATGAGCTCTAT
AAAGGCATGAGCTACATGGCCCCACAGAACTTCCTGCACACATTCGAAGCCGAG
GATTGTATCGTGGCCTTCAACTTTGCCAATGAGGAAGAGGCCCGGCACATGAGAT
ACGTGATTCTGGAGAAGCAGAAACGATTGGAGAGAAGACACCGGGCCTCCACTC
AGCCTCGTCACTCCTCCACCCCGGGCCTGGACAGAGAGCGAAGCCGAACCCTGC
AGCCAGCCGCCATGACCAACGGGACCAAACTGTCGCCCGCGGAGAGAGCAAGA
CATGTGCGATCCTCGTCCGGCGGAGGAGGCGGAAACCGGAAGAGGGAGGCCAA
GCGGCGAGGCAACAAGCTGACGAAGGCGGACATATCCTCCCCCACGGGGTTCAG
ACATGTGTCCCACGTGGGGTTTGACCCCAACAAGGGGTTCGACGCTGTGGACATT
CAGAACAGCCCCGAGCTGGAGATGTTCTTTGAGAAGGCGGGCGTCTCGCAGAGT
CAGCTGCAGGACCGCAAGACCCGCGAGTTCATCTACGACTTCATCTCCCGCAACG
GC (SEQ ID NO: 20)
#P-96 BcGS_05363 Cortactin
ATGTGGAAATCAGCAGCAGGTTCTGCAATAGCACCAGTTGTTCCGGACGAAGAT
GATGACTGGGAAACTGATCCTGACTTCATCAATGATGTAAGTGAACAGGAGCAG
AGGTGGGGCTCCAAAACTATACCAGGTTCTGGTAGAGATGCTGGGTCTATTGACA
TGAAGCAACTGCGGGAAGAAGTGGCTATGTCAGATGCATGCTACAAGCAAAAGC
AGTTAGATGGAGGATCAAAAGCTTCTTTTGGATATGGAGGAAAATTTGGTGTTGA
GAAGGATAGGATGGATCAGTCAGCTGTAGGACATGACTATGTCGCACAGCTTCA
TCAACACGAGTCTCAGAGTGATTACAAAACTGGGTTTGGGGGGAAGTTTGGTGT
GCAAAATGATAGAGTTGATAAAAGTGCTCTGACTTGGGAGCACAAAGAAGTAAT
AGAAAAACACACTTCTCAAAAAGATTATAGTTCAGGATTTGGAGGTAAGTTTGG
AGTGCAAAAAGATCGACAAGATAAATCAGCAGTAGGATGGGACCATCAAGAAA
AGATTGAGAAACATGAATCACAAAAAGATTACGCTAAAGGATTTGGTGGTAAGT
TTGGCATAGAAAGTGATAGACAAGATAAATCAGCAGTTGGATGGGACCATGTAG
AGAAAGTAAACAAACATCAGAGTCAAACAGATGCTAACAAGGGTGTTGTAAGTT
CTTCAAAAGTCAAAGAGCTGATAGCTGCCAATTCCAATACTTCCATTAAAGAAAA
TGTCAAACCAAAACCTGACATCAGTCATGTGAAACCATCCAACCTAAGAGCAAA
ATTTGAAAACTTGGCAAAACAAACAGAAGAAGAAAGTAGAAAAAGAAGTGAAG
AAGAAAAAGAAAAAAGAAAACTAAAGGATCAAATAGATCTCCAACAGGCTCAA
AAGTTAGAGGAAAGACGTCTATCAGAATTGCAAGTAAAAGAAGCTGAAGTTGAG
AGAAAAATGAATGCGCACTCAAATGTGCCTTCTTCACCAACAAGTGATTCAATTC
CAGTCAAGTCAATACTAAAACAATCATCAATTGAGAAAATAACTGTTCAAAATA
GTAATGATGAAGAGAAAGAGAAACAAAAAATGATTCAAGAAGAAATTGAGAAG
AAAAATGAGCTTGAAAAAGAAAGAATAAAGCAAGAACAAGAAATTAAAAAAAG
AAAAGATAAAGAAGAAAAAGATAGACAAGAAAGAGAGCAACAAGAAAAAGAA
CAAAAGGAAAGAGATGAAAATCAAAAATTACTCCTTAAAAAACAGCAAGAAGA
AGATAGACTAAAAGCTGAGGAGCAAGCAAGACTCTTGGAACAAGAGAGGCTAA
TGGAAGAATTAAGACTTCAAGGAAATGATGATAACACAGAGGAGGATTTGGGCT
ATACAGCAATAGCACTTTATGATTACCAAGCATCTGCTGATGATGAAATTTCTTT
TGACCCTGATGATATTATCACTAACATTGAAATGATAGATGAAGGCTGGTGGAG
GGGCTTGTGCCATGGACAGTATGGGTTATTTCCAGCTAATTATGTTTCCCTCCAAC
AA (SEQ ID NO: 21)
#P-115 BcGS_00175 Delta-24 sterol reductase
ATGCTTTCTGAGGCCACCCTCGAGCACTTGCTCAAGAACTACCGCTGGGTCCTCG
TGATATTCGTCCTACTGCCTCTCTCCCTCCTCTACGACATCTACCACAGCGTAGGA
CAGCTCATCACGGagtactgcagggacaagagccaggaccatggcaagaaagtgaaccatgttcaaagtcaggttcga
gcctggctggcaggggggcagacctcccccatgtgcaccgccagggctgggtggaagagcatgaccctgcgggagcccaagtac
aaggcgaccatgttccctgtggacctgggacctctggattccatcctctctgttgacgaacacagtcatacggttctcgttgagccttacg
tgaccatgggtcagctgacacgctacctcattCCCAAGGGCTGGACAATTCCTGTGGTTATTGAGTT
GGATGACGTCACTGTAGGCGGCATTGTGTCTGGACAAGGCCTGGAGTCGAGCTCT
CACAAGCATGGTCAGTTTCAGAATACATGCGTCTCCTATGAGTTGGTCCTCAGTG
ATGCCAGTGTGGTCCAGTGTAGCAAGGAAAATGACCCCGATCTTTTCTACGCTGT
GCCTTGGTCTTATGGAACTCTGGGATTTCTGACGGCCGTCGAGATACAACTTATT
CCCGTTAAAAAATACGTTCAGCTCCAGTATGTGGCTCTCAAGTCCCTGCCGGATC
TGGAACATCACCTGAAGAAAGAGGCAGAAAACAAAGGCAACGACTTTGTGGAA
GCCATTGTGTTCTCCAAAGACCAGTCTGTCCTTATGATAGGCACCTTCTGTGATAC
CCCGGAACCAAGCAAAATTAACCGCCTGGGTCGCTGGTACAAGCCTTGGTTCTAC
CAGCATGTGAGGAGTTACCTTAGCAGGAAGAAGTACGCGGAGGAGTACATCCCT
ATCCTTGACTACTACCATCGCTTCAGCACATCCTTGTTCTGGGAAATACAGGACA
TTGTCCCCTTCGGGAACCACCCTCTCTTCCGCTACGCCCTCGGCTGGCTGATGCCC
CCCAAAGTGTCCCTCCTCAAGCTGACCCAGACCCAAACCATCAAGCAGCTGTAC
GACAAGCATCACGTGGTGGAGGACTATCTCCTGCCCCTGGGAGAGGTGAGGGCG
TTTCTGCAGCATATCCATGACCAAATACAGGTCTACCCTCTCTGGATCTGTCCTTT
CCTTCTCAAGGATCTTCCCGGCCTTGTACATCCTTCCAAGCCTGGGGATTGTCTCT
ATGTGGACGTGGGAATATATGGAGAACCAAAGGCGCAGGATTATGACAGCAAGA
AAACCATTCTGGATGTTGAGAATTATCTTGGCAAAATTAGAGGATTTCAGATGCT
CTACGCTGGTTGCTATGAGTCCAGATCTGAGTTCCGGCACAACTATGACCACAGC
CTTTACGATAGCGTCAGGTCCAGGCTGGCCTGTGAGAAAGCGTTTCCCGTCATAT
ATGATAAGGTGAACCGTGGTGTCCGAGATTGA (SEQ ID NO: 22)
#P-116 C-7 cholesterol desaturase From PoP Genome:
PGA scaffold2_1222_contigs_length_49276144:9,297,307-9,335,888(~)Partial ORF
(Rev Com)
TTGCAGGACCTGAGTGAGGTGGGCTACGGACACCTGGACTCTGCCAAGAAGCGT
GGCGCCCACAACCACTACACGCGCGGCTACGGGAAATCCAAGGCGCACATCATC
AACGAAATGCGACGGGCAAGGAAAATCGGCAACCTACCGCCCGTCTACCCGAAC
GGTTGGTTCGCGTTGatggaatcaagcgagctgcgacctcgggacgcgaaatacgtttcggcgctcggggagaatttc
gccgtgttccgatcggaatcgggcgaggtgcatgtgctggatgcctactgcccgcatttgggcgccaacatggcgatcggtgggttcg
tgcggggggactgcatcgagtgtcccttccaTCAGTGGCAGTTCAGCGGACGCGACGGCCGCTGTG
TGAACATTCCTTACAGCGGAAAAGTGCCGGAGGTGGCTCGTGTGAGGCACTGGC
AGTCAGTGGAAGTGAATGACTTTGTGTTCGTGTGGTACCACGCAGAGGAGGAGG
ACCCATCCTGGCAGCCGGAACCACTTGACAAAATCACAAGAGGGGACTGGCGCT
ACCGGGGCAGATCAGAGTACCTCATCAACTCTCACATCCAGGAGATCCCAGAGA
ACGGCGGGGACATCGCCCATCTGAACGCCATCCACGCCCCTTCCCTCGTGGCGGG
CAGCAACCTGCACGACCTGGAGACCACCGCCGCCCAGTCCGCCCGCCACGTGTG
GCAGGCCACCTGGGAGCCGCATACGGCGGCCGGAGAAACACACGTGGCCACCAT
GCGGCTGCGGCATGACCTGCGGCTACTCGACCGCATCCCGCTGCCGCTCATTGGC
ATGAACGTCGAGGCCCGACAGGTGGGCCCGGGCTACGTGGAAATGATCATGACC
ACAAGCATAGGTCGCCTCGCCATCCTCCAGACAGTGACTCCGGTGGAACCTATGC
TGCAGAGAGTGATTCACAGGATCTACGCCCCGCCCCATCTCTTCTGGTACGCCAA
CATCGTTCTCTACGGGGAGTGCATCATGGTGAGTCGAGACATCATGGTGTGGAAC
CACAAGACCTACATAGACAAGCCCCTCCTGGTGAAGGAAGACAAGACCCTGGCC
CGACACAGGAGGTGGTACAGTCAGTTCTACACCGAGAACAGTCCCCGCTACGAG
TCCAAGAAGGATACTCTGGATTGGTGATAG (SEQ ID NO: 23)
#P-160 BcAN_01048 RAC
AATTAATTTAATTCACTTCAATTTAATTAATTATCATTGTTCTAGTACAATAGGTT
TACCAAAAACACTTCCACCATGCAAGCAATCAAGTGCGTTGTGGTAGGAGATGG
TGCCGTGGGTAAGACCTGTCTCCTCATCAGCTACACCACCAACGCCTTCCCCGGC
GAGTACATCCCCACCGTATTCGACAACTATTCGGCCAATGTGATGGTGGACGGGA
AGCCCATCAACCTCGGCCTCTGGGATACGGCCGGCCAAGAGGACTACGACCGAC
TTCGGCCCCTCTCCTACCCGCAGACTGACGTGTTCCAAATCTGTTTCTCGCTCGTG
AACCCGGCCTCGTTCGAGAACGTGCGAGCCAAGTGGTACCCCGAGGTGCGGCAC
CACTGCCCCAACACGCCCATCATTCTGGTGGGCACCAAACTGGACCTGCGGGAC
GACAAGGAGACCATTGAGAAGCTCAAGGAGAAGAAACTGGCGCCCATCACATAC
CCCCAGGGCCTGTCCATGGCGAAGGAGATCGGGGCCGTCAAGTATCTGGAATGT
TCGGCCCTCACTCAGAAGGGCCTGAAAACTGTGTTTGACGAGGCCATCCGCGCA
GTGCTGTGTCCCGTACCTACTGTTCCCAAGAAGAAACGGTGCGCGATCCTGTAAT
CGAACCCGCGGTCACCAGCATACAGAGATGAAGCCCCCGCCTACCACCGCAGCC
CTTGTGTAAAATATCATGTCTTTGACTCGCCAGTATTTGCAGGCGCGGCCGGTGG
GAAAATGTTTTGGGGGTGCTGCCACATGAACCGAACTAAAATGGGATTTATAGA
GTCGCTCGCTCAGTAATGGGTTTGTTTACCA (SEQ ID NO: 24)
#P-165 BcAN_01762 Crc, Cryptocephal
ATGATACAGAAGTCTCCATCTATACAAATCAGGATGACTTCACTAGGATTATGGA
GTCTGGAAGGTACCCCAGGTATCTTGGAAGTCTATAATCATGTAGACTCCCCACC
AGAAGACCAAGGAGATGACACGAGCAGCCACACGAGAGCGGAGGTTGCCTCCA
AACTATTGGAAACACTTGACAATTTCAACTATGATGAAACAGAAGAGTCGTCGG
ACGCCTACATCTCCGACTGGCTCACCGGCTCGGAGGCGGCTGTGGCTCTGCCCTC
CATCTTCGAAGACCTCGCCTCCCTGCCCCCCCCGCCTCCCCTGCCCCTAGTGCCCA
CGGGCCGCCAAGCAGTCTGCTCCTCAGGCCCGGGCGGGTTCTACGGGCCCCGCG
GCGCATGCGCAGTAGTCCCCACTGTCCCCGTGATCGACAAGCTCGGGTACGGAG
CGAGTGCGGCGCTAGTATGGAACTCGAATAAGAAGGTTGGCTACGAAGCGACCG
CCTTGTGGACGCCGGCGGGCTCCGATGACTATTCCACCGGCTCCCTCTACTCTCC
GCCCGCCGCGGGCACCCAGTGGCACCTGGAGCCGGTGTACCAGGGCGTGTCCAA
CCAGCTGACGCCGCCGCACAGTCCGCCCACCATGTACGAGACCAGCCCCCGCCG
CCCGCTCGAGTTTGATGATCTGCCAGCAGACTTGCTGAAATCATCTGCCGCCAAG
GAGCTCTACGAAGACCTGCCTGCCGAGTCCGCCCTCAACAGCAAGGAAGAAAAC
AACAACGGCCAGCTGATCATGTCCCTGCTGGCGGAGATGGACCAGAAGGACATT
GACGAGATCGTGCAGTGCTCCGTGGAACAGAACTTCGGAATCGCGCCTGATTCC
GAACCCAACTTGGAAGCATCCGCCGCGACCAACTACGTCGAATCGCTGAGTCCC
GAACACTCGTGCTCTTCATCCGAATCCAACTTCGGCTATCATTCCGAGTCCGACC
GAAGCTCGTCGTGCAGCTCCGACCCCGACTACAATCCCTACTCGCCGCCCGAAGT
GACGCGAAAAACGCCCGAAACAAGCCGAAGTGGCGGTGGCGGACGAAAAGCGG
CGAAAACGAGCGAAGCCAACCGAAGTGCTCGAGCGATCAAACCGTACGCCCGCA
AAGCGGCCGTGCCCGTGGAAGACAAGAAACTGCGCAAGAAAGAGCAGAACAAG
AACGCGGCCACGCGCTATCGAATCAAGAAGAAGGCGGAGATCGAAGTCATTCTG
GGCGAGGAGTCGGAACTCAGGGAGAAGAACGAGGAGCTGCAGAAGAGTGTGGA
GGATTTGAATCGCGAGATCAAGTTCATGAAGAAGTTCATGCGCGACTTTTTCAAA
ACGCAAGGTGTGCTCAAGTGA (SEQ ID NO: 25)
#P-178 Chitinase 7 BcAN_02808 Length: 2961 Chitinase 7
ATGATATTGTGGAAAGATGGAAGAGCATTCAGTGAAATATTATTCCTATTGTTAG
CCATTATATTCCTAGCTTCCTCCACATACAGTACACAGACTGTGAGGAGGCGGCT
GCGGCGGCCCTCCATCAAGTCCAAGCCGTCCGAAGAGTCCGGGGACCAGGATGT
GTCCGCCAGCATCAACAGGTTCAAGCTCAAGAGCCGCACCTCCGCCTCCAGCAG
CGCCGTGGAGGCGGCCGCCAGCGGCAAGTCCAGTGTGAAGGCGGGCAAGGATG
ACCCCAAGTACAAGGTCGTCTGCTACTACACCAACTGGTCCCAGTACCGCACCAA
GATCGGCAAGTTCCAGCCCGAGGACATAGAGGCTGACCTCTGCACGCACATCAT
CTTCGCTTTCGGCTGGCTCAAGAAGGGCAAGCTGTCCAGCTTCGAGTCCAATGAC
GAGACCAAGGACGGCAAGGTGGGGCTGTATGAGAAGATCGACCAGTTGAAGAA
GGCCAACCCTAAGCTGAAGACGCTGCTCGCCATAGGAGGATGGTCGTTCGGTAC
CCAGAAGTTCAAAGAGATGTCCAAATCCAGATACTCCCGTCAGACCTTCATCTAC
TCCGCTGTCCCCTTCTTGAGGCAGAGGAATTTCGACGGCTTGGACATTGATTGGG
AATACCCCGCCGGAACTGAAGACAAGAAGAACTACGTACTCCTCCTGAAAGAAT
TGAGAGAAGCCTTCGAAGCGGAAGCTCAGGAAGTGAAGAAGCCCCGCCTCCTGC
TGACAGCTGCTGTCCCTGTGGGCCCCGACAACATCAAGGGAGGATACGATGTCC
CCGCCGTGGCCGGCTACTTGGACTTCATCAACCTGATGGCGTACGATTTCCACGG
CAAGTGGGAGCGGGAGGTGGGCCACAACGCGCCCCTGCGTGCGCCCAGCACCGA
CAGCGAGTGGCGGAAACAGTTGAGTGTCGAGAACGCCGCCAACATGTGGGTGCG
TCTCGGCGCCCCCAAGGAGAAGCTGATCATTGGAATGCCCACCTATGGGCGCAC
CTTCACCCTGGCTAACCCCGCCGTGTTCAAGGTGAACAGTCCCGCCACTGGGGGA
GGCAAGGCAGGAGAGTACACCAAGGAGAGTGGTTTCCTCGCTTACTATGAGATT
TGTGAAATGCTGAAGAACGGCGCAGGCTACGTCTGGGATGATGAAATGAAGGTC
CCCTATCTGGTGCATGGTGACCAATGGGTGGGCTTTGATGACGAGCGATCCATCC
GGTACAAGATGAACTGGCTGAAGGACGGGGGGTATGGAGGTGCCATGGTGTGGA
CAGTGGACATGGATGACTTCACTGGAACAGTGTGTGGAGGAGACGTCAAGTACC
CGCTCATTGGAGCAATGAGAGAGGAGCTGAACGGGATCCCCCGAGCTAACAATG
CCAAGGATGTGGACTGGGCCAAGGTAGCCGGCACCCTGGACGTAGAATCCGCCA
CTAAACCCGCCGCCATCAAGATCTCCGTGGCGGATGTGCTCAGTAAGGCCAAGA
AGATCACCATCCAGAAGGTCGACACCAGCAACTTGATCGATTCCAAAGTTCGCC
CCGCCCAGATCTTCTGCTACATGACCAGCTGGTCCCAGAACCGACCCGGAGCTGG
CAAATTCGCCCCAGAGAACATTGACCCCAAACTCTGCACCCACGTGATCTACGCC
TTCGGAACCTTGAAGGATCACAAGCTGACCTTGAACGAGGAGAAGGAGAAGGAT
GAGGCAGGCAAATTCGAGGCCCTGATGCAACTGAGAGAGAAGAACCCTGATATC
AAGATCCTGCTAGCTATCGGAGGCTGGGCCTTCGGCTCCACCCCCTTCAAGGAGC
TGACAGGCAACACCTTCCGTATGAACCAGTTCGTGTACGAGTCCATAGAGTTCCT
GAGGGAGCACAAGTTCAATGGCCTGGATGTGGACTGGGAGTACCCCAGAGGTGC
CGACGACCGAGCTTCCTACGTGAACCTGCTCAAGGAGCTTCGTCAAGCTTTCGAA
GGCGAGGCCAAGACCTCCGGTGAGCCCCGGCTCCTTCTCACAGCCGCGGTGCCG
GCTAGCTTCGAAGCCATCGCAGCCGGCTACGACGTGCCCGAGATCTCCAAGTAC
CTGGACTTCATCAACGTGATGACCTACGACTTCCATGGCCAGTGGGAGCGACAG
GTCGGACACAACAGCCCGCTCCAACCTCTGGAAGGTGCCACCAGCTACCAGAAG
AAGCTGACAGTGGAGTACAGCGCCAAGGAGTGGGTGAAGCAGGGCGCCCCCAA
GGAGAAGCTCATGATCGGTATGCCCACCTATGGGCGCAGCTTCACTCTGGTGGAC
CCGAGCAAGTTCGACATCGGGGCGCCAGCCTCAGGGGGCGGCGACCCTGGCAAG
TACACAGCCGAGGCTGGGTTCATGTCTTACTACGAGGTATGCGACTTCCTGAAGA
AAGACAACACTACCCTGGTGTGGGACAATGAGCAGCAGGTTCCATTCGCCTACA
AGGATGATCAATGGGTCGGATTTGATGATGAGCGCAGTCTGAAAATGAAAATGG
ACTGGCTGAAGGAAGACGGCTACGGAGGCATCATGATCTGGAGTGTGGACATGG
ACGATTTCAGAGGCCAGTGTGGCTCCGGCAAGTACCCTCTGATCTCCACCATGAA
GAGCGAGCTAGATGACTACTCCGTGGCTCTGGAGTACGACGGACCCTATGAGAC
CTACAGCCCCACCGGCAAATACACGACCAAAGAACCCAATGCCGTGTCATGCGA
GGAAGAGGACGGTCACATCTCCTATCACCCGGACAAGGCCGACTGCACCCACTA
CTACATGTGTGAGGGCGAGCGGAAGCACCACATGCCCTGCCCCTCCAACCTGGT
GTTCAACCCCAAGGAGAACGTCTGCGATTGGCCCGAGAACGTAGAGACCTGTTT
ACACCACACCCAAGCCCCGCCTCCATCCAAA (SEQ ID NO: 26)
#P-181 Endochitinase A-like BcAN_08176 Length: 4728 Endochitinase A-like
ATGAGCACCCTGCGACAGCTACACCTGCAGGACTCGGACCCCGAGGAGTTCTCC
GAAGACGAAAGCTCCCCCTTGTCTCCCAACGACATCTATGGCGGCAGCACCCGC
ACAGTGCCCGAGACGAAGGCTTGGGATGTGTTCCGGGACCTGCCCCCCCGACAA
GACTCGGGGTCCATGGCCTCCAAGGCCTGCCTGGAGTTCACAATCAAGGCCCTCA
AGATCTTCGTCTACCTCTTCACCTTTGTGGTGGTCCTGCTCTCCGGGGTTGTGGCC
AAGACAGCTGTGTTCTTCATGACGTCACAACTCCGCGCCCCCGACAAGCACATTC
CCTACTGTAACAAGGAGTTTGGTCGAGACAAACAATTCGCGGTGTACCTGCCTTC
AGAGGAGCGGATCGCCTGGTACTGGTGTCTCATCATAGCCTTTATAGTCCCCGAG
TTGGGAACCGCTGTCCGGTCTATCAGGATCTGCTACTTCAAGTCGTGGAAGAAGC
CCCCACTGCACGACTTTATCTTCGTCATCACCATGGAGCTGCTGCATACGCTGGG
TCTGGCCTGCTTGATGTTCGTCCTACTACCCAACATGGATGTGGTGAAAGGCGCC
ATGCTCACTAACTGTCTCTGCTTCCTGCCAGCTTTCCTCACGCTGATATCTCGCAA
CACCAGGGGCCTAACCAAGGGGTCAGAGAGGTCCGAGGTCTATCTGAAAGCCAT
GATGGATATGATAGCCGTGTCAGCCCAGGCGACAGGGGCGCTGCTGTGGCCTCT
CCTGGAGGGGCACAATGACCCTTGCCTCTGGTTGATTCCACCGGCCCTGTTCTGT
GTGTCCTGTGGCTATTGGGAGAACTATACAAGCAAACATTCCATTTTCGGGTTCA
TGAAGAGCGCCTGGCGAGTGAAGGAGAGACTGAAGCGCACGCGCTACTTCACCT
ACGCCTTTGTGTCCTTCCTCAAGATCCTAGTCTTCTTCTGCTCGGCGCTGGTGTTC
ATGTCCTTCCGCGGGGAGGGGGTGGGGGAGTTCTTCTCCAAGTTTCACGCCTCCT
TCGCCCAACACAAGATCAGGATCTACGAGGTGAAAGCCTCAGCGTTTGGAACTT
CTATCCCGGACCTGGCTGATCCAAACCTCACGGGAGACTACCGGGATATTGATTC
TGAGGATAATTTTGCCCTCAAGGTCCTGCTTTTCCAGATATTCGCTGCCTACCTAT
GCTACATTGTGGGCAAGTACGCGTGTAAGATCATGATCCAGGGGTTCAGCTACGC
GTTCCCCGTGAACCTCACCATCCCCGTCACCATCTCTATCCTCATCACCATGTGCG
GACTCCGCAATGACGACCCCTGCTTTTTCCAGAACTCCATTCCCGACTACCTCTTT
TTCGTGTCTCCCTCCATTTACTTCCTCGACGACTTTATTATCAAACAGCATGCATG
GGTGTGGTTGTTATGGTTACTGAGTCAAACTTGGATTACGCTACACATCTGGACA
CCAAAATGTGAGAGGTTGGCCACCACGGAGAAATTATTCGTGAGACCCATGTAC
GATTCTTTGCTTATCGATCAGTCTATGAGTATGAACCGTAGATGTGATGATGAGA
AGGACGTCAAAACTGAGGAACTCGTGGATCGCGAGAAAGACCCTGACGAATACT
ACGAGACAATCTCCGTTCACACTGACGCTTCCTCCACTACACCCAAGACTGTCAA
GAAATCCGACTCCATCACGCGCATCTACGCATGCGCCACAATGTGGCACGAGAC
CAAGGACGAGATGATGGAGCTGGTGAAGTCTATCCTGCGGCTGGATGAGGACCA
ATGCGCCCGCCGAGTTGCGCAGAAATATCTGCGGGTTGTTGATCCGGACTATTAC
GAATATGAAACTCACATTTTTTTCGACGATGCTTTCGAAATCTCCGACGTGAATG
ACGAGTGGGCCCAGGTGAATCGCTTCGTGAAGTTGTTCGTGTCGGTGATAGATGA
CGCAGCCGCCCATGTACACGAGACCAACATACGCATCCGACCCCCCACCAAGTA
CCCCACCCCGTACGGAGGGCAGCTCATATGGACCCTGCCGGGGAAGACCAAGAT
GATCGTACACTTGAAGGACAAGGCGAAGATTCGACACAGGAAGCGGTGGAGTCA
GGTGATGTACATGTACTATCTGCTGGGCCATCGTCTGATGGAGCTGCCTCTGCCT
GTGGACCGCAAGGAAGTGATGGCCGAGAACACCTACCTGCTCACTCTGGACGGC
GATGTGGACTTCCAACCCGACGCTGTGAGGCTCTTGGTGGACTTGATGAAGAAG
AATCGAAACCTGGGCGCTGCCTGCGGGAGAATCCATCCTGTTGGTGGAGGACCT
ATGGCTTGGTATCAGAAGTTCGAGTACGCCATCGGTCATTGGCTTCAGAAAGCCA
CCGAGCACATGATAGGCTGCGTTCTGTGTAGTCCGGGCTGCTTCTCTCTCTTCAG
AGCCAAGGCTCTCATGGATGACAATGTGATGAGAAGATACACCACCAAGTCTGC
AGAGGCCCGGCATTATGTGCAGTACGACCAAGGTGAGGACCGATGGTTGTGCAC
GCTACTGCTGCAACGAGGCTACCGTGTGGAGTACTCAGCGGCCAGCGATGCCTA
CACCCGGTGCCCCGAGAGCTTCAATGAGTTCTACAATCAGCGGAGACGGTGGGC
CCCTTCCACCATGGCTAATATCATGGACCTGCTCATGGACTATAAGAAGACCATA
AAGATAAACGATAACATCTCGATGCCCTATATTTGGTACCAGGTGATGTTGATGG
GAGGTACCATCCTGGGCCCCGGCACTATATTCCTCATGTTGGTGGGGGCCTGCGT
GGCAGTGTTCCACATTGACAACTGGACCAGCTTCTACTACAACATCACTCCTATT
CTCTTCTTCATGTTTGTTTGCTTCTGCTGCAAGTCCAACATACAGCTCCTGGTGGC
TCAGATACTCTCCACAGCATACGCCCTCATCATGATGGCTGTGATAGTGGGAACA
GCTCTACAACTGGGGGAGGACGGTATTGGCTCTCCGTCTGCCATTTTCCTCATAG
TCATGTTGGGGTCCTTCATCATAGCGGCATTGCTTCATCCGCAAGAGTTCTTCTGC
ATAGTCTTCTTCTCCATTTATCTCTTATCTGTGCCGTCCATGTACCTTCTTCTCATT
CTCTACTCACTCATCAACTTGAACGTTGTCTCTTGGGGTACTCGAGAGGTCGCAG
TGAAGAAAACCAAGAAGGAACTAGAGGAGGAGAAGAAGCAAGCCTTGCAGTCC
AAGAAGAAAGCCAAGAACCCTCTCCTGGGCTTCTTGCAAAACTACACGCCCGAC
GCTGGGGAGGATGAGGAGGGCAGTCTGGAGCTTTCCTTTGCTGGACTCTTCCGCT
GCATGTTCTGCACCTACCCCAAGCCGGTGGACGAGAAGCAGCAACTTCTCCGTAT
AGCGGACAGCTTGAGTGGACTGGAGAAGCGACTGGATCACATTGAGAGGATCGT
GGACCCCAACATGCCCAACCCTCATCACCACAGGCGCCGCACTACATCAGCCAG
CAGCAGCGCGCACCATGCACCACTAGGCTCCGTGCCAGAGAACAAGACCGAAGA
TCAGTCCACGGACGAGGAGGATGAAGACAGTGATACTGTGTCCACAGCAGAGCC
CAAGATTGAGCGTAACGATGACATTAACCCTTACTGGATCGAGGATAAGGAATT
AAAGAAGGGTCCTGTAGCATTCATCTCACCTAATGAGAGACAATTTTGGGTAGA
ACTCATTGATAAATATCTCTACCCCATCGATAACGACAAGGCGGAGCAGAGCCG
TATAGCAAGTGACTTGAAAGATCTACGCGACCAAAGCGTGTTCAGTTTCTTTATG
GTGAACGCTCTCTTTGTTGTGATCGTGTTTTTGCTGCAACTCAATAAAGATAAGTT
GCACGTTCGCTGGCCCTTTGGAATCAAAACGAATATTACGTTCAATGAGGCGACA
CAGGAGGTGATCATTACTAAAGAGTACCTACAGCTAGAGCCGATTGGTCTAGTGT
TTGTGTTCTTCTTCGCCCTCATTCTGGTCATTCAGTTCACTGCCATGTTGTTCCATC
GGTTCGGAACCATCTCGCACATTCTCGCGTCAACGGAACTCAACTTGTGTTGCAA
CAAAAAGACTGAAGACCTGTCTCAGGACGGGCTCCTAGACAAGCAAGCCGTGGA
CATCGTCCGTCACTTGCAGAGGTTGCGGGGCATCAACGGAGAATATGAGAACGA
CAGTGGCTCCTCAGGTGACCGGGTGGGGAGGAGGAGAACCATCTACAACCTGGA
GAAGCAGAAGAACAAGACAAGGACGATAGGAACTCTGGACGTGGCGTTCCGGA
AGAGATTCCTCCAGATGAAGATGGAGGATGTGGTGGAGACAGCAGGTACACCAG
TGCTGGGGCGCAAGTTGACCATGAGGAGAGAGATACGAGAGGCTTTGGAAGTGA
GGCGTCGCTCCTTACAAGCAGAGAGACGAAAGTCCAACATGCAAACCCTAGGCG
CTCATAACGATATTTATGGACGAACTCAGAGGATCTCAAACGCGGGATCTCACGT
GAAGGACTTGTTTGACGGGCATCAGAACCCTGCGTACGAGCCTGACCTCGCCTCG
GACAGTGACCGACCCCTGCCCGGGGGGCGCGCTGTCAACTGGGCGGAGCTGGAG
AGTCAGAGGATCAACAGTCACATC (SEQ ID NO: 27)
#P-184 Chitinase 5 BcAN_12316 Length: 1473 Chitinase 5
GTGGGGGGCTGGGGGGAGGGGGGAGAGAAGTATTCCGCCATGGTGGCGGACAA
GAGCAAGAGGACCACCTTCATCAGGAGCATCGTGGGTTTCATGAAGAAGTACAA
CCTCGATGGTTTTGATCTGGACTGGGAGTACCCTGCTGCTACAGACAGAGGTGGC
AGCTTCAGCGACAAGACTAACTTCTTCTACTTTGTGGAGGAGCTGAGGACCGCGT
TCAACAGATTTGGGAAGCACTGGGAAATCACCATGGCAGTTCCTATGGCCAAGTT
CAGACTCAATGAGGGATACGATGTGCCTGAACTTTGCGACCTAGTGGACGCAATT
CATGTGATGTCGTATGATCTGCGAGGTAACTGGGCAGGCTTTGCCGACGTGCACA
GTCCTCTGTACAGGCGACCTTCCGATCAGTATGCTTACGAAACGCTCAATGTGAA
TGACGGACTCCAGCTGTGGGTGGACAAGGGGTGCTCCCCTCGCAAGCTCGTGGT
GGGTATTCCGTTCTACGGGCGGTCCTTCACTCTGAGCAACAGCAACAACAACTAC
AACATAGGCACCTACATCAACAAGGAGGGGGGGGAGGCGACCCGGGCCCGTA
CACGAACGCCACCGGCTTCATTTCCTACTATGAGATTTGTTCAATGGTGGAGAAA
GATCATGATTGGGTACAAAAATGGGATGACATTGGAAAATGCCCTTACACATAC
AAAGGAAATCAATGGATTGGCTACGAGAACCCAGAGAGTGTTCAAATCAAAATG
GACTGGATCAAATCCAAAGGATACGCTGGAGCTATGAACTGGGCAATTGATATG
GACGACTTTAATGGACTTTGCGGACCCAAAAATGAACTGCTGAGCATTCTGTACA
GGAACATGAAGGACTACAAAGTGCCCATACCGCAGGTCTCGATAACACCAAGGC
CTGAATGGGACAGGCCTCCCAGCACAACACCAGAGGACATAGACTACGCCCCTA
TTTACCCTCCAGTAACCACGAGCAAACCTGTTCTTGCATCGACTTCCCCTACTAG
AAAACCCGTCCTTTCAACCGCCTCTCCCACCGCGCCCGGAGTTTCCAGCACAGAA
AACTCTGCTGTGATAGAGGCGACCACTCCCCAGAACAAACCCCACAAGAGGCCA
CGCCCTTCCATCACGGCTAAACCCCCCAGAGCACATCACCGAGGCCGGAAGAAA
CCTATCCGGAGACTCCTGAGGAGAATCATCCTGAAGGCAACTTGCCAGAACCCA
GTGGAGTAGATTGCTCCAGCGATGGTTTCCAAGCTCATGCTCTCTGCAATAAGTA
CTACCAATGCGTCTTCGGAAAACCCATAGAATTCACATGCCGACCTGGTACGTAC
TTCAATAGAAAAATGAGCGTCTGTGATTGGCCAGAGAAAGTCGATGTCAATAAA
TGCAAAATGATCAAAATTCTGAAGACGGTGGACTACGAAGATGCAAACCAATTT
TTG (SEQ ID NO: 28)
#P-190 BcGS_06785 WAVE
ATGCCTCTGCCCAAGCGAGTGATAGAACCTGTCCACGTAGCCCGAGGCACCATC
CCTGATGAGCTGTCCGCTAGTCTACCTTCGGAACTTGAGGCCGCCACCAATGGAA
CCCTGGCTAACACGGTTCGTCAACTGTCCAGCCTCAGTCGCCACGCGGAGGATAT
GTTCGGCGAGCTCACGCGGGAAGCCCACGGCATGGCTGTGAGGGCCAACTGCTT
GCAGGTGCGGCTGGACCGGCTGGCTGTCAAGGTCACGCAGCTAGACAGCACCAT
CGAAGAAGTGTCCCTGCAAGACATTCATCTGAAGAAAGCGTTCAAGTCGGCCAT
CGTGTTTGACCAGGAGGTGGTGTCCCGCAGTACTATNNCAAGAGGTGGTGTCTCG
CAGCACTATGCCCACAGCCATGCTGGAGACCTACAAGCAATG (SEQ ID NO: 29)
#P-192 BcGS_20515 Brain-specific angiogenesis inhibitor (BAI) 1-associated protein 2
CAAATCGTGGAAGTATGGAACGAAATCCAGTCACAACAGATGAACATTCTGAAA
GCCTTCTACGTTCTCATGATCGTGCCTCTGGAGACCAACCTGGAGAAAGACACCA
AGGTGGTGACGGCGGAACAGAAGAGGTTCCTGCAGCAACACAAGATCCTCTCCG
AGTCCTACAGCAAAGCCGCAGCCAACATGAAGAAGCAACGCAAGAAACACAAG
AGCAACTCCAAGGCGAGCAACACAGTGAGCAACAAAGAGATCAAGTCCATGCA
AATTCTGGAGGAAGAGAAGACAAAGCTGGACGCATTCTGTGAGCAGGGATTGAA
AAACGCGATGACTCAGGAGCGGCGCCGCTACGGCTTCGTGCTGGAGCGTCTGTC
CAGCCTCGCCAAGCACTACCTGGCGTACCACTCTGCCGGGGCCACCCTGTATGAG
AGGAACATGGAGGCGTGGGGGGAGGTGAACAAGACCCGGGAGGCGCTGCCTGA
GAGTGTGGAG (SEQ ID NO: 30)
Sequences: Asian citrus psyllid (ACP), Diaphorina citri
#A-7 Dcitr06g09110.1.1 ACP v-ATPase-A
ATGTCAACTGCATTAGGAAAAATGGCTGATGAAGATAGGGAAGGAAGGTTTGGG
TTTGTGTATGCAGTCTCAGGTCCTGTGGTAACTGCAGAAAAAATGTCAGGATCTG
CTATGTACGAACTGGTACGAGTGGGATACTTTGAACTGGTGGGAGAAATCATCA
GATTAGAGGGTGACATGGCTACTATTCAAGTGTACGAAGAAACATCGGGTGTGA
CTGTCGGTGACCCTGTGCTAAGGACAGGCAAACCCTTATCTGTTGAGCTTGGTCC
TGGTATCCTGGGAAGTATATTTGATGGTATTCAGCGTCCATTGAAAGACATCAAT
GAGTTATCTCAGAGCATTTACATCCCCAAGGGTGTCAACATTCCTGCATTGAACA
GAGATGTTAGTTGGGAGTTCAACCCAATGAATCTGAAGATTGGAAGTCATATCAC
TGGTGGAGATCAGTATGGTCTTGTTCATGAGAATACACTTGTCAAGCACAAGATG
ATCATGCCCCCTAAAGCTAAGGGTACTGTCACTTACATTGCACCTGCTGGTAACT
ATAAAGTTGATGAAGTTGTTCTTGAAACTGAATTTGATGGAGAGAAGAGTAAAT
ACACCATGGTTCAAGTATGGCCTGTCCGTCAGCCCCGCCCTGTCACAGAAAAGCT
ACCTGCTAACTATCCACTATTGACAGGTCAAAGAGTTCTTGATTCCCTCTTCCCCT
GTGTTCTTGGAGGAACCACTGCCATTCCAGGTGCCTTTGGTTGTGGTAAAACTGT
GATTTCCCAGGCTTTGTCCAAATATTCCAACTCAGATGTTATTGTGTATGTAGGAT
GTGGAGAGCGAGGTAATGAAATGGCAGAGGTACTGAGAGATTTCCCTGAGCTTA
CCATTGAAGTTGATGGAGTTACCGAATCAATCATGAAGCGTACTACACTTGTAGC
CAACACATCTAACATGCCTGTAGCTGCCCGAGAGGCTTCTATCTACACTGGTATC
ACACTGTCTGAGTACTTCAGAGACATGGGTTACAATGTGTCCATGATGGCTGACT
CTACATCCCGTTGGGCTGAGGCTTTGAGAGAAATTTCAGGACGTCTTGCTGAGAT
GCCTGCTGACAGTGGTTATCCTGCCTACCTAGGAGCCAGACTGGCCTCATTCTAT
GAACGTGCTGGCAGAGTCAAATGCTTGGGTAACCCAGACAGAGAAGGTTCTGTG
AGTATTGTGGGTGCTGTATCTCCCCCTGGTGGAGATTTCTCCGACCCTGTCACTTC
TGCTACTCTTGGTATTGTCCAAGTGTTCTGGGGTCTTGACAAGAAACTTGCACAG
AGGAAACATTTCCCCTCTATCAACTGGCTCATCTCTTACAGTAAATACATGAGAG
CCTTGGATGACTTCTATGATAAAAATCATCCGGAGTTTGTACCTCTGAGAACCAA
GGTAAAGGAAATCCTTCAAGAAGAAGAAGATTTATCAGAAATTGTGCAACTGGT
TGGTAAAGCCTCCTTGGCAGAATCTGATAAAATCACCTTGGAAGTTGCCAAGTTG
CTGAAAGATGATTTCCTTCAGCAAAACAGTTACTCACCCTATGACAGGTTCTGTC
CCTTCTACAAAACTGTGGGAATGCTGCGTAACATGATTGCTTTCTATGATATGTC
CCGCCATGCTGTTGAGTCTACTGCTCAGTCAGAAAACAAAATCACATGGTCTGTC
ATCAGAGACAGCATGAGCAACATTCTGTACCAACTTTCCTCCATGAAATTCAAAG
ACCCTGTCAAGGATGGAGAAGCTAAAACCAGAGCAGACTTTGATCAACTGTATG
AAGACATTCAGCAAGCATTCCGTAACTTAGAAGACTAA (SEQ ID NO: 31)
#A-8 Dcitr09g08730.1.1 ACP v-ATPase-B
TGTCGATAAGCTCGAAGCAAGCTTTACGGGAGAATGTGCTAGCTGTGACGCGGG
ACTACATATCGCAGCCACGAATAACATACAAAACTGTGTCTGGTGTCAACGGAC
CTCTGGTCATCTTAGACGAGGTGAAATTCCCCAAGTACGCTGAAATTGTGCAGCT
CCGTCTGAATGATGGATCTTACCGTGCCGGACAAGTGCTGGAAGTCAGTGGCTCC
AAGGCTGTGGTCCAGGTGTTTGAAGGTACATCTGGTATTGATGCTAAGAACACAG
TTTGTGAGTTCACTGGAGACATCTTGAGAACTCCTGTGTCAGAGGATATGTTAGG
ACGAGTGTTCAACGGCAGTGGAAAACCCATTGATAAAGGACCTCCCATCCTAGC
CGAGGACTACTTGGACATTGAAGGTCAACCCATCAACCCGTACAGCAGAACCTA
CCCGCAGGAAATGATACAGACTGGTATCTCAGCTATCGATGTGATGAACTCTATT
GCTCGTGGACAGAAGATTCCCATCTTCTCTGCTGCTGGTCTGCCCCACAATGAAA
TTGCTGCTCAGATTTGTAGACAAGCTGGTCTGGTAAAGATGCCGGGTAAATCTGT
ACTTGATGATTCTGAAGATAACTTTGCTATTGTGTTTGCCGCTATGGGAGTCAAC
ATGGAAACTGCCCGATTCTTCAAACAAGATTTCGAAGAAAACGGTTCCATGGAG
AACGTGTGTCTGTTCTTGAACTTGGCCAACGACCCTACCATTGAACGTATCATCA
CACCTCGACTTGCCCTCACCACAGCAGAGTTTTTGGCGTACCAGTGTGAGAAACA
CGTGTTGGTTATCCTTACGGATATGTCCTCTTATGCTGAAGCTTTGCGTGAGGTGT
CAGCTGCCCGTGAAGAAGTACCAGGGCGACGTGGGTTCCCCGGATACATGTACA
CCAACTTGGCTACCATCTATGAGCGTGCTGGGAGAGTGGAGGGCAGGAACGGAT
CGATCACTCAGATCCCTATCCTTACTATGCCTAACGATGATATCACCCATCCTATA
CCCGATTTGACCGGGTACATTACCGAGGGTCAAATCTACGTCGACCGACAGCTGC
ACAACCGACAGATCTATCCTCCAATCAACGTGCTCCCCTCTCTATCCCGTCTGAT
GAAGTCTGCTATTGGTGAAGACATGACTCGCAAGGACCATTCAGATGTGTCCAAT
CAGCTGTACGCTTGTTACGCTATTGGTAAGGATGTGCAGGCCATGAAGGCTGTAG
TAGGAGAAGAAGCTTTGACTCCAGATGACTTGCTGTACTTGGAGTTCCTCACCAA
GTTCGAAAAGAACTTCGTGTCACAAGGTAACTACGAGAACCGCACGGTGTACGA
GTCCCTGGACATCGGCTGGCAGCTGCTCCGTATCTTCCCCAAGGAGATGCTCAAG
CGTATTCCCGCCTCCACGCTCGCCGAATTCTATCCCCGGGATTCACGCCACACTG
GCGCCAAGTAA (SEQ ID NO: 32)
#A-9 Dcitr09g02100.1.1 ACP v-ATPase D
ATGTCAGGTAAAGAGAGACTACCCATATTCCCTTCCCGAGGAGCTCAGTCACTCA
TGAAATCTCGTCTGAAGGGAGCTCAGAAGGGACACAGTCTTCTCAAGAAGAAGG
CTGATGCTCTACAGATGAGGTTCCGAATGATTCTCAGCAAGATTATTGAGACTAA
AACTCTCATGGGAGAGGTAATGAAAGAAGCTGCGTTCTCTCTAGCAGAGGCCAA
GTTCACTACTGGAGATTTCAACCAGGTAGTTCTACAAAATGTCACCAAGGCACAG
ATCAAAATCCGCACTAAGAAAGACAATGTAGCAGGTGTTACTCTACCCGTGTTTG
AGAGCTATCAAGATGGCACAGACACTTACGAACTGGCTGGTCTAGCTAGGGGTG
GACAGCAGCTGGCAAAGCTGAAGAAAAATTACCAGACAGCTATAAAACTCCTCG
TAGAACTTGCTTCACTACAAACATCATTTGTAACATTAGATGATGTCATCAAAAT
TACTAATCGGAGAGTAAATGCCATTGAGCATGTCATTATTCCTCGCATTGAAAAG
ACACTAGCATACATTATTTCCGAGCTGGATGAGTTGGAAAGAGAAGAGTTCTACC
GTTTGAAGAAAATTCAAGACAAGAAGAAGGTGATCAAAGCAGCTTCTGAAGCTT
TTAGGAAGTCTCGTAAATATGATGAAGAACAGGCATTCAATATGCTAGAAGAGG
AAGATCAGGACATTTTATTCTAG (SEQ ID NO: 33)
#A-11 Dcitr09g02500.1.1 ACP v-ATPase E
ATGGTCCAAAATGTATTACAGAGCAACAAAGTTCTGAAAGATTTTAGTCTTAAAC
TTTGCGACAAATCTACATTCCGGTCGTACCTGAAACTTCCGAACGCAGCCGACAT
TTTCCGACCGCCCCCGACACTTCTCGGCATGGCGTTAGACGACGCAGCGGTGGAA
AAGCAAATCGAACGCATGGTGGCGTTTATCCAAACCGAGGCGGACGAAAAGTTG
GACGACATCCGGCGAAAAATCGAAGAGGACTACCAGATCGAAAGAGAGCGAGT
AACGCGAGACGGAAAGGCGAGCGTGGATGAAGAATATGCTAAGAAATACCGCC
AGGTAGAGCTTCGGCACAGGACGGACTGTTCCAATATTAAGAGCGAAGGACGGA
TGAATGTCATGAGGGTGAAAGAAGACAGTGTTGGAAAGATCATCGAGGAAGCAA
AAGGCAGACTGTCTGACATTACAGAGGATCGCACAAAGTACACCGAGATACTCG
AAAAACTCATTTTCCAAGGTGTCCTCAAGTTACTGGAACCCACTGTTCTGATTCG
ATGTAAAGAAAACGACCTGTGCATTGTGAGGCAACTACTGCCGCTAGTAGCTAG
GGACTTTGAGAATATTACGGGATCCAATCTTACGCTGCTGATCGATTCTGAACAG
TACTTACCACCTGAGATTTCAGGAGGCGTAGAGCTTATCACCCCCGATGAGAAGA
TCAGAGTGAACAATACGTTGGAAATGCGACTAGAACTGATGGCCCTACAAGCTC
TGCCCGATATGAGAGATATATTGTATGGTCCTAATCCGAATAGGAAGTTCGACGA
TTGA (SEQ ID NO: 34)
#A-30 Dcitr01g19610.1.1 ACP Actin
ATGTGTGACGATGATGTAGCCGCTTTGGTCGTGGACAATGGTTCCGGTATGTGCA
AGGCCGGATTCGCCGGAGATGACGCTCCCCGAGCTGTCTTCCCCTCAATCGTCGG
TAGACCCCGTCATCAGGGTGTCATGGTGGGTATGGGTCAAAAAGACTCCTACGTC
GGTGATGAGGCTCAATCCAAGAGAGGTATCCTCACCCTGAAATACCCCATTGAG
CACGGTATCATCACCAACTGGGACGATATGGAAAAGATCTGGCATCACACCTTCT
ACAATGAGCTCCGTGTTGCCCCCGAGGAGCACCCCATCCTGCTGACCGAAGCTCC
CCTCAACCCCAAGGCCAACAGAGAAAAGATGACCCAGATCATGTTTGAAACCTT
CAACACCCCCGCCATGTATGTTGCCATCCAGGCTGTGCTCTCCCTGTACGCTTCC
GGTCGTACCACTGGTATCGTGCTTGACTCCGGAGATGGTGTCTCCCACACCGTCC
CCATCTATGAAGGTTACGCCCTTCCCCACGCCATCCTCCGTCTGGATCTGGCTGG
TCGTGACTTGACCGACTACCTGATGAAGATCCTCACCGAGAGAGGTTACTCTTTC
ACCACCACCGCTGAGCGTGAAATCGTTCGTGACATCAAGGAGAAGCTGTGCTAC
GTCGCCCTGGACTTTGAACAGGAAATGGCCACCGCTGCTGCCTCCACCTCTCTGG
AGAAATCCTACGAGCTTCCCGACGGTCAAGTCATCACCATCGGAAACGAAAGAT
TCCGTTGTCCCGAAGCTCTGTTCCAACCTTCCTTCCTGGGTATGGAATCTTGCGGT
ATCCACGAGACCGTGTACAACTCCATCATGAAGTGCGATGTTGATATCAGAAAG
GACCTGTACGCCAACACTGTCCTGTCTGGAGGTACCACCATGTACCCCGGTATTG
CCGACAGAATGCAAAAGGAAATCACTGCCCTGGCTCCCTCTACCATCAAGATCA
AGATCATTGCTCCCCCTGAGAGAAAGTACTCCGTGTGGATTGGAGGTTCCATCTT
GGCTTCTCTGTCTACCTTCCAACAGATGTGGATCAGCAAACAAGAATACGACGAG
TCCGGCCCTGGAATCGTTCACCGCAAATGTTTCTAA (SEQ ID NO: 35)
#A-34 Dcitr01g15580.1.1 Arp2/3 complex 34 kDa subunit
ATGGGTGATCAAGAAATGCCAAATTATATAAAGTTTTTGTTCGGAGGAACTGCAG
GGATGGCAGCCACAGTATTCGTTCAGCCCTTGGACTTGATCAAGAATCGAATGCA
GCTGGACAAAGCTAAAGAATACCGGAGCAGTATCCAAGCATTCACAACCATCCT
GAGGAAAGAAGGAGTGTTTGCCATGTACAATGGTTTGTCTGCAGGTCTTCTGAGA
CAGGCAACTTACACCACTACAAGACTGGGAACCTATAATTTACTGCTAAACAAGT
TCAAAGCGGAAGACGGAACCCCGCCCAATTTCTTTCAAAAAGCTGCGTTCGGCAT
TGCTGCCGGCGCCACAGGGGCTTTCGTAGGCACGCCCGCTGAGGTAGCCCTTATC
CGTATGACTGCAGACGGTAGACTCCCTCCAGCCGAAAGACGTAACTACAAGAAC
GTATTCCACGCCCTGTACAGGATGACATCGCAAGAAGGTGTTCTCACACTGTGGC
GAGGAACAATACCTACCATGGGAAGAGCTATGGTGGTCAATGCAGCTCAACTAG
CTTCGTATTCTCAGGCCAAACAGTTTTTGTTGAGCTTGAGCTATTTCGAAGAGAA
CATTTTCTGTTTTTTCGTGAGCAGTATGATATCTGCCTTCATCACTACGGTAGCTT
CTATGCCAGTGGACATTGCCAAGACCAGGATTCAAAACATGAAAACGATCGACG
GCAAGCCAGAATTCAAAGGAGCGTTCGACGTGCTCGGCAAAGTCATAAAACAAG
AAGGTGTCCTTGCCCTGTGGAAAGGTTTCATGCCCTACTTCCTTCGAATAGGTCC
GCACACGGTACTGACCTTTATATTCCTCGAACAAATGTGCATCTTCTATAACAAA
AAGTTTATGGGAAACACCTCAGGCAAGGGGAGCGGTATACTGAAACCAGAATCA
GTGGATATTACAATAGCCGATTTTGATGGGGTACTATTTCACATTTCAAACTTAA
GTGGTGATAAAACAAAAATAAGGACTAGTATATCACTCAAGTTCTACAAAGAGC
TAGAGGAACATGGTGCTGATGAATTGTTGAGGAGAGAGTATGGATCATTGCTGT
CTGAACCAGAGCCAGGTTACAATGTGTCCGTACTCATCAACCTGGAAGATGTGCC
CTCAAATTGGGAGGAGATAGTCAAGAAAATAGGTCTCTTAAAAAGAAATTGTTT
TGCATCTGTGTTTGAAAAATATTTTGACTTTCAAGAGAGAGGGGAAGAAGGTCAC
AGTAGAGCAGTCATCAATTATAGAAATGAAGAAACCATGTACGTAGAGGCGAAG
GCAGATCGCGTGACGGTCGTATTCAGTACGGTATTCCGAGACGAAGATGACGTC
ATCATAGGCAAAGTATTCATGCAGGAGTTCAAGGAGGGGCGACGCGCCAGTCAC
ACGGCCCCTCAGGTGCTGTTTAGTCACAAGGAGCCCCCACTTGAGTTGCGAAACA
CAGATGCCAGACAGGGGGACAATATTGGCTACATTACGTTTGTGCTGTTTCCAAG
ACATACGAATCGGGTGGCTCGAGAGAACACCATCAATCTGATTCACATGTTCCGA
GACTATTTGCACTATCACATCAAATGTTCCAAAGCTTACATTCACTCCCGGATGC
GAGCGAAAACGTCCGACTTCCTCAAAGTGCTCAATCGCGCCCGACCCGAAGTCA
AAAACACAGAGAAGAAGACAATCACTTATCAGATGATGAAGTCACTGCCATGTT
TAGAACAACTCTGCCCTAGGTGCCACCCCTACTCCATCTTGTGCTCAATGTCACT
GGAGTCAGCACCTGGACTTCTTGGAATGGCTAACTGCACGAAATCAATATTGGAC
CCATTGATGTGTGGTAAGATGGAGGTGGACAGTGCTATCGATTTAGAGATAGAT
GAGATTCTAGAAAAGCCTCTGTTTGGGGTCAGAGTGTGGGAAATATTGTACGGA
GCTTTCGCCGTCATTTCCCTCATAGCTTTGGTTCGAGTCAAAGGAGTGTGTGATGC
TGAAAACCAGCCCAGTCCGGGAGGTTCTAGTCAGTGTGAAGACCTTTGTGATGAT
AATCATGATACCCGGAGAGACGAACAAATGAAGCTGCACTTGAGAAAACTGGAT
CAGAACAAGCACAAGTTCAAGTTTTCCAACATGATCAAATCTCTGAATGTATTCC
CGTCACGCATGCGACATTCAGCACCTGCTCTAAATGTCAAGCCAAGGGAATTGCA
AGTGTAA (SEQ ID NO: 36)
#A-93 Dcitr05g17000.1.1 ACP Wiskott-Aldrich syndrome protein (WASP)
ATGCCTACTAATGCACCTCCTGAACAAAAAACATCTAGTCTCTTAACTCGGCAAG
AAAATGAGGCTGTGTTCAAGTTGTTGGGTAACAGATGTCAGGCACTCTCAACTAC
TGTGATACAACTGTTCACTACCGATGGACCAAATGATAACGAGTGGCACAAGAG
ATGCTTTGGTATCCTATGTCTTGTCAAAGATAACCCAAGGAAATCCTACTTCTTCC
GTTTATACTGTCTGACTAGAAAACAGTTGCTGTGGGAACATGAGCTTTATACAGG
TATGGATTACATGGCACCTCAGAATTTCCTGCACATATTTGAAGCTGAGGATTGT
GTTGTGGCATTCAACTTTGCAAGTGAAGAGGAAGCCCGCCACATGAGATATGTG
ATATTGGAGAAGCAGAAGAGGATTGAGCGTAGACACAGGGCCTCTACTCAGCCG
AGACATGCTACCCCCGGGGCAGATCGCGAGAGAAGTAGAAGATTACAGCCTGCT
GTTCTTACCAATGGAACTAACCTCACACCCAAGTCTAGACATATGAGATCTTTAT
CCGGTGGCAGAGGCGGTGATAGAAGGCGAGATGGGAAGAAGAATAAACTTACC
AAGGCAGACATATCATCACCCACTGAGTTCCGGCATGTATCACACGTTGGATTTG
ATCCCAATAGAGGATTTGATGCTGTTGACATTCAGAATAGTCCCGAACTAGAAAT
GTTCTTTGAAAAGGCAGGCGTCTCACAAAGTCAGCTGCAAGACCGGAAGACCCG
AGAGTTTATTTATGATTTCATATCTCGTAATGGCGGTCTGGACGCTGCTAAGGAA
GAGGCGCACCTTCCCCCACCCCCCGTACCTGCTCGCTCTGTCCACCCGTCCAACC
CTCGCCATGCCCCCCCTCCACCCCCGTCCCGTAATATACCGCCTCCCCTGCCATCC
CTCCGTACCCTCCCCTCCCGCGCCCCTACCGCGCCACCCAAAGACATGCACATAC
CCCCTCCCCCGCCTCCACCCCCCTCTCTACCACCCCCTGCTACCATACAGCAGAG
TACACCCCAGCCTGCCCAGCAACCTGATCCTAGAGCTGCTCTCATGGACGCCATA
CGATATGGAAAACAACTCAAGCCTGTAGATCCAGATTCTAAGAAGTCTACTCTGA
CCAATGATTCGAGAGGAGAACTGTTAGATCAAATCCGACAGGGAGTCGAACTGA
AAGCGGTTCAACCCGTATCCAAGCCAGCAGTGAGTACACCATCTGATGGTTTAGC
CGGAGCTCTAGCGCGCGCCCTGGCGGAGAGATCTCGAGTTATTCATAGTGATTCC
GATGACACAAGTGATACAGATGAGGAAAATGAGGATGAAAACGACGAGGATGA
ATGGGAAGACTGA (SEQ ID NO: 37)
#A-96 Dcitr04g02520.1.1 ACP Cortactin
ATGTGGAAAGCAGCTGCAGGTTCTGGAGTAGCTCCCGTTGTTGCAAATCAAGATG
ATGATGACTGGGAAACTGATCCAGACTTTATTAATGATGTGAGTGAACAAGAAC
AAAGATGGGGTTCAAAAACAATACCAGGCTCAGGTAGAACAGGAGGTACTATTG
ATATGAAACAGTTGCGAGAAGAAGTTGCCATATCAGATGCTTGCTACAAACAGA
AACAGTTGGAGGAGGGATCTAAAGCATCTTATGGATATGGTGGTAAATTTGGTGT
TGAGAAGGACAGGATGGATGAGTCTGCTGTCGGACATGACTATGTCGCTCAGCTT
CATCAACATGAGTCTCAGAGTGATTACAAAACTGGATTCGGAGGAAAATTTGGA
GTGCAGAATGATAGAGTGGACAAAAGTGCTGTTACTTGGGATCATAAAGAAGTA
ATAGAGAAACATGCTTCTCAAAAAGATTACAGCTCAGGCTTTGGGGGGAAATTT
GGAGTACAAAAAGATAGACAGGATAAATCAGCTGTGGGATGGGATCATCAAGA
GAAAATAGAAAAGCATGAATCTCAGAAAGATTACGCTAAAGGTTTTGGAGGTAA
ATTTGGCATTGAATCAGACAGACAAGACAAATCAGCTGTTGGTTGGGATCATGTA
GAGAAAGTAGACAAGCATCAAAGCCAAAAAGATGACAACAGACAAGTTGTGAC
TTCATCTAAAGTAAAGGACCTAATAGCAGCTAATTCCACAAATGCTTCCAAGGAA
AATATTAAACCGAAACCAAATATTGGTCATGTTAAACCTTCTAACCTGAGGGCAA
ATGAAATCAATCCTCAAACAACCAACTAG (SEQ ID NO: 38)
#A-115 Dcitr04g03460.1.1 ACP Delta-24 sterol reductase
ATGGCTTGCGATCGTTGTGTTCAACTTTCATCGGAAGTTGAAAAATTGACCACTC
AAAATAAGAAGATTGTACAATTGCTGAATGTGTTAAATGCCTCCAAGAAACACA
AAGAACAACTGATAGAACATACCTTGGATTACATCAAGTCCAAAGATGAAGACT
ACAAGAAGGAAAAGATCAAAATCAAAGAACTTGCAATCAAAGTCAAGAACACC
ACCAAAGAATCGGTTCTCAGGAATGTACCCAAGCAGATTGATGAGTGCAGTGCA
CTCTACATCAATAAAACTGCTGAGATACAGAATTACATTAGAGCCACAGAGATT
GCCAGAGCTGCCAAGCAAGAGTTGGAAACAAAGAGCCTTGAATTGGAAGGAAA
CAAAATGTACGTGAAGCAACTGATTGCCAAAGTTGATGAGCTAGAACATAACAA
GAGGACTTTGACGGAATCCTTGGCATCAACTAAACTGCTCGTCACAAAAGCTTTG
ATGAGAAACAAGAAGATAGAGCTTGAGATGAGAATGCTCAATGATGCTCACTCA
TATCATAACAGAGTGGAAACCGGAGTGAAGAAAGCTTTGCGTAGATTGGAATCA
TCAGATCCAAATTCCACAAAGAGCTGTATGAGGATCCTGAATAATCTAGTTCATT
TCTTTGAAAAAGAACCACTCTTTGTGAAACGATCTCGGAAATTGAAGAATGGTGC
GGATAATAAGGGCATAGTCAGTCCTCACAGTGTATCTTCTGGTTATAGCAGTGGA
ACTGCAAGTCCTTGGTCTACTCCTTACAATACTCCTCAATCCTCTGAGGAACCTA
ACACTCAGTTCACATTTACCTTCACACCGGACGGGGATTGTGTGCTCTCCCCTCTT
CCGGCTCAGTCACAGTCAAACCCTTCAACTTCATCCTCATCAACCCCCACCACCA
GGTCTTCCATTACTTCACCATTGGTTCAACCATCTCCACCTCATTCCAGCTATGAA
CCAAGTCAGAAACTTAGTTCTGAGAAAGTCGCAACCTCTTGGTCAATGTCTTGTG
ATAGTTCGGATGATGAAATTGTTGATGGAGCTGATGATCGGGATAGTGATGTTGA
AAGTGATCGAGAGACCTCTATAAAAAACACTTGTAAATTAGAAACCAATAAACA
CAAAAGTAAAGACCTTGGTGATTTAGCAAGTGACAAAGTCAAGGAAACTAGGGA
ATTACAAAGTGATAAAGGCAAGTCGTCACCAAAAAGAGGTGAACTTGACAGTGA
TATGGTTGAAATTGGTGAGCGGAGCAAGAAACCATCCAGTGATAATGTACAAAG
TACATTTGATCTTGTTGAGCCAAATGACATGTGTGAAAGTAACAAAATCGATTGT
GATAATAGTGATGAAGGGAAGAGCAGTGATATCCAGGAGATAACTATAGCAAGT
GAAGTAGCCAGTCCCAAACATGCTTCTGCAGAGTTACTACACCTTCCTGTCCTTG
AGAATTTTGAAGATGAAAAACCTATAAAACAATTACCTGGAGTTACAAGTGCAG
AAAAGGATCAAGCCCTTCCAAAGCAAACTGTTCAAGCTAATCATAGTCCAGAAG
CTAAAAAACATTTCTTTGGTTCGTATAAGCAGAGTTTGTTTTCACCTTCAAAGAC
ACCACCGGGAAAGAAGGAAACAAAACAGGAGATTGCAAGGGAAATTGCCAAAG
CGGCCAAACAAATAGAAATGAGTCTCTGTGAAGAAGAGAATTGTGTGAAGGAGA
AGAAGAAACGTGTATCACGAAGTGTGAAGAGAAGGAGCTCTAAAAAGTCTATAG
GCGATGATGAAACTGACGCAGAAAGTGCTACTGTGGTTGCAAAAGAAAAGAAAA
ATGGAACGTCTGAAATGAGCTCAGAAGATGACAGCTTGGAAAAGTTGCGTGTGT
TGAGAAAATCTCGTAAATCAATTGACAAAGCTCTCAAGGATAATGTCGGTAGTA
CTAAGTCTTCTGGAAAATCGCCAAGACGTAGATCAACTAGAAACAAAAGTACAG
AAACCAGTTCTATTCGTTCAGAAACTAGCTGTGTCAATTCAGAAAACAATCTTAT
TGACTCTACCAGCACAGACTTTGAAATTTCCAACCTCTCAGACAGTGATTGTGAA
AGTACTGATCTTGTAGACAAAGCTGATGAGAACACCGAAGAAAATGTCAAGGAA
CTTTTGAAAATCGACAAGGAACGACACGAACACGTGTTACAGATATTCAAAGAC
ATTGAAGTGATGCAAAATGTTGAACTCATTTCGAACTTTGATCTTCTGGCTTCACC
CTGCAAGAAACAAACGCGATCCAGATCCAACAGTCTAGTTCCAAATAACGAAGC
AGATAAACTAGAAAATGATGTAATTCCTAATGCATTTGATTGTGATAAAGATATC
AGCAATACAGAACTTGAGAAAGAGAAATATGAATCACTCGAAGCTGTATATAAC
GAAACCAACAAAAAGCTCGTGAAAGATTCTGAAGAAAATGCCCAAGATGATGAA
GATGATTGTAATTCACAAGTAGAAGATGATGAATGCAAAACGCCCTCTTTGAGA
AGGAGTAGAAGAAACTCAAAACGGAATGCAGAAGAGATATCCAGTCCTGTAACT
AGGAAGTCACCTCAGACCCAAAACACCGAGTCCAATTTGAAAGGTACCCCCTCT
GACAACAAGAAGTCACGAAAATCACGCACCATCGAAGAAGAAAGGTTACCAAG
AACGAGATCAAAATCCACGCACAAGGCAGTATCTATTCCTGAAGTAGATAATTT
GAGACAAAGAAGCTCAAGTGACGTTGGAACAAAGAAGTCCTCTGAAGAAACGG
AAATACCACTTGCCGATAGTGATGAAATACCTAGAAGAGTAACACGAAGAAGTC
TATCCGTCATGGAGAACAAAGAAGATGTTAAATATTCAGAAAAAACAAGAAGAG
GACGTAATAAGAAGGCGGACAATAGTTTATTAGGAGAGGCAATGTCAGATGGTG
ACAAGGATAAGATTAGTTTCGAAGATAAGTGTGATAAAAACAGTAAAGACAAAC
CGAATTCTAGTGACTATAATGGAAAAGATCAAGTTGTTCCAAGTGCTAGCAATGA
AATTGAAAGTGTTGATGATCTTGATGAATCTATGGAAATAAACGATATTGATGCT
TCTGTTGAAACATTATCAGAGATAACTGATAAATCTAGTGGAATAGACACAGAG
AAAAATCTCGTGAAACAAATAGAACCTGAGAAAGAAGAAATGGTAGACGACGA
CAAATCAAGTAATCTTGAAAGAACTCTATTCGAAAGTCGTGTAGAAATTACAGA
AAGCAATAAAGAAGATGTAGAATGCAATGACAGCATGGTACAAGATGTGCGATG
TTTGAAGAGAACTCGAATTGCCCTATCTAAAGTCGGTGGTGATGCCGTGTCCAAA
GTATCCAGTGATACACTTGATACAGTATCCAAAGTCAGTGATGAAAACAAGGCG
CTGTCTCCTCCAGAGAAGGCTGTTAGCTATCCTTTGGAATCGAAGGAAACACCAG
ATAAATCTGCTCCCAACACTGCTCCATCGACAGCTCAACCTATGTCACCTCAGAA
TGATAAAAAATTAGAAACAGATGCCCCGAAAATACCTAAACCAGTTTCAGTTCTT
GAGGAACAATCTTCGAAGATATCTGATCCAAGTCCTGTTCTGCAGTTAAGTACCG
GGAGAAAGTTCAAGATTGGACGCTTGAAAGAGGTATCTAACACTACCAATGAAA
CAGCAGTCCCTAGGAAAGAATTGGCCGATAGCAGAAATATTGAGACTAGTGTTG
AATCCAGTAAGGAATCGCCAAGTACTCCCAGTAGTGGAAAAGAACATGTGGAAC
TAGCAAATGAAAAAGTTATTGTTGGTAAAGAAGATCAAATCCACAAGATCAGAT
CGAACAAAAACATTTTGAATACTAAAAATGAGAAAGTACTTGCTAATAAAAAGG
ATACAGTCCCCGAGGATGTCTCTACAAATGAAAATGGCCAAAACATTCTAAACC
ATACAGTCAATAAAGCTTCGACAAAGAATACTATACTACCTATTGTCAAAACTGA
CAACTTAATGCAACAAAACAGAATCATCCAAAGTTACAATTGTAACTTGAACCTG
GTCAAACCAGCCACAGTACAGAAACCTCCTCATACAATCGTGAATAGTAAGAAA
GTCAATAGAATTCTCCGAAGAATGGGAATGGAGGGATTCAACAAGAAGTTTCCA
TTCATTGACGCTCTCCCTGCTACTCCACCTCAGTGTAGAGAAAATGGAACTGGAT
TCAAGAACTACAATGGGTATAACAACTCTACTATTGCGAAGGAAGAAGTTGAAG
AGATGGAAGAAACAGATTGTTTGGTTATGGAGATGGAAGATGACTTACAAGATG
TTAGGGAACCAAAAGATGCAGATAGTGAAGTATCTGAACAATTGAGCTCGCAAA
CTACATGTGAAGATAAAGTACCTGTTCCAGATATGACACAACCACTTGCAGATAA
AGAAGAAGTAGTAGAAAAATCGATCAGCTTGCCTAGGAAAGAGGGAGAAGAAT
TAAACCAAATGCCAGTACCTATTCCGAATATAACAGATATGACACAGCCACTTGT
GGAGAAAGAAGGAGGAGTGGAAAAGTCACCCAACAGATCTCAAAGAAAGGATG
AAAAATTAAACTTCGTCAGCAAAATAATGGAAAACAGTCTTCACAAAGAAGATC
AAATCCAAAACAAAGGAATTGTTCCTGAGCAAAGCACAAGCTTCCCTAATGTAC
ACGTCCCAGACAAAGAGTCTACTAGTCCCAATAAAATGAATAATGACTTGAGTC
CTAACAAATCAGATGACACAACCAGCTTGACATCCACAACATCTAGTGGATTGA
AGTCTCCTTCAAACCTGTTGACATCCAATGAAACACCTTGCCAATCTTTGCAGTC
ACCTCCCTCAATATTTCCCAAATGTATTCAAAACCGCTTGGCAAAGATCCGTTCC
ACACACAATTTACCAATTTATAACATAACTCCAATGAGTAATTTGGCTCACAATA
CTCGACCTGGGGAGAAAACCAACATGAATTTAGGTGTTCATGGACTCAAGTCGA
ATGAAATGAGCCCTCCAAGTCTAAACAAAGTAGACATTCAAGATGCCGTCAAAC
CAGTTCAAGCTAGTCCTACAGATATTTCCAATCAAGTGTCCTATAACAATAATCC
AGAACCGACTCTTGTTGGAAACCAAACTTTTAGCTTCTATGGACAGAAGAGATCT
TCGGACATTACTGCTCCTTTTGAAAAAGAAAGCACGGTATTTAGACCACCAAATA
ACTTCAGTAATACTAACACGGTTTCTAGACCATCAAATAATGTCACCTCTAATAG
TGACAATATGGTATCTAGACCACCAAATAACTTGAACACTAGTTTCGTCAACAAT
AGCCCTCCTAAGGTCAACGGACCTTTGGAGAATTCTGTTCATCAGGCGAATGCTA
CCAAACCCACGGAAAAGATTGTTCTTCCAACATTATCCTTCTACAACACACGCAA
TTCTCCTTCATATGGTGCTAACAAACGTAAATGTGAAGACCCTGAATATGTGAGT
TCTGATGAAAGCAAGAAACTTAGAACTGAGCTTGAAACCAGTGGTACATTGAAG
AGCACGGTTAACACACCTAAAGACAAAGATGAACCCAAGTCCTATACACTCATT
CAACGCTCAATTGAACATCTACAATCCGAAATGTCCAAACCCGATGTGACACCA
ACCAATCAGATCAAAACTAAAACGGTGACGAAAAAACTGAACCTGCTGTACTAC
GCCATGCAACCGAATGATAAGAACGTGTTCGATAGGAAAATCAAAGCGTTTGTC
AAAGAGCTGGAAGATTACCCGCTGGATACTTTATCCAAACGAATCATCGCGTTGT
ACGATAACAAACAGCTGGTAGACAATCCCAAAGCGGTGACAAACATTCAAACCA
AAGTGTTCTGTCTGATTATACAGCTAAACACAACACGGCCTGAGTTCCTACCCAC
GTTTATGAAGACCCTGCATGAGACACTGGTCCTGCATAGTGAGACCACACTCCTA
TCTATCCTGTATGCATTGACACAACTCTATGTAGGCTTGTGTCGCTGGCGTAATG
ATCCAGCATCTGTTATGACATTCCTCTACGATACCATCTACTTCCATAGGAAACG
CTCTCTCAATATACTAGACGCTTTGATCACACGATGGCCTACTGTGTATCCTGTAG
CCAGTCGCTGTGTCCTAGTTAAAGTAATGAGTTGTATCCTGCTCAACCAGCAGAC
ATCAAGCCAGGGGAGTTTCCTTGATGACCAGAATGTCTTCGTGAAGACAAAGCA
GCAGTTGAGAACAAAGTACAGATATCAGGAACAGTTCACACTTGCCGGTCTACT
AGCAGAGTTGATGAATATGTTGAAGAGTAAACACTCCTGCTGCTCTAACCCGTGG
CAGCGAAACCCTCGTTCCAATTCTCACCACCAGGGAGCGGAATCGTCGAGTTTCA
GCGGTTGTGTACAGAGGGCGCTGGTGCTGTTGGCTAAACATTGGCGTCCGGCGCC
AGACTTGTACAGTGTTGTCGTGGAGAGGAACTTGGCAACACTGTTCAGAAGTAGT
TTGGATTGTGGAGCAATCAGTGTTGCTGCCTCCTGCGCTGAACTCATGGGTATGA
TTGGGAAAGCCATGCCCCGCGCCTCCTCCCTCCTCTGCATCGTCTCCCTGCTGGAT
ATTCTGGAGGTCGGCCTGCAACATGCCGCCTGCCGCTCACCCTCCCCCCTTCGCA
CCTCTATAGGCCGCGCGCTAACCTGCCTCACGGCCCACAACACGGAGCGTTGCGT
GGCCATCCTCACCTCGGTGCCTTGGACAGAGGCCGAGGTCAATGACCTTGTCAAG
GACGTTGCAGCCAAGGTCATCGCGTCTTGTAAGATCCGAGTGACAAGCACCATG
ATTTCCGAATCTCTCCTAGAACACCTGCTCAAGAAGTACCGCTGGATTTTCGTCA
TTTTCTTCCTTCTGCCCCTGACTTTCTTCTACGATATCTATCACTTCATACGACAGC
AGGTCACCGAATATTTCAAGGACAAATCAGTATGCCATGATCTCAAAGTAAAAC
ATGTTCAAGGTCAGGTTCGTGAGTGGATCAAAACCGACCAATCCATACCGATGTG
TACAGGACGCGCTGGGTGGAAGTGTATGTCACTGAGAGAGCCCAAGTACAAGTC
GTCCATGTTTCCAGTGGATCTGGAGGCGATGGATACCATCCTGAGTGTGGACGAG
GAGAAGAAGACGGTCAAAGTAGAGCCCTATGTGACCATGGGTCAATTAACCCGC
TATCTAATACCCAGAGGCTGGACCATCCCTGTCGTGGTAGAACTCGATGACGTTA
CAGTAGGAGGCATTGTTCTGGGTCAAGGTCTGGAGTCAAGTTCTCACAAGTACGG
GGTGTTTCAGAACACATGTGTCTCCTATGAACTCGTACTGAGTGATGCCAGTCTG
GTTGAATGTAGTGAGGAGGAAGACCGGGATTTGTTTCACGCTATACCTTGGTCCT
ATGGAACACTGGGATTTCTGACGGCTGTTGAAATTCAAATCATTCCTGTAAAAAA
ATATGTTCAGCTTCAATATGTCGCGTTGAAATCTCTGCCAGATCTAGAGCATCAT
CTGAAGAAGGAAGCGGAAAACAAGGCCAATGATTTCGTGGAAGCCATCTTGTTT
TCCAAGGACCAGTCCGTGCTCATGATAGGTACCTTCAGTGATTCGCCAGATCCAA
GTAAAGTGAACAACCTAGGCCGCTGGTATAAACCCTGGTTCTATGAACATGTAA
GGAGCTACCTTACCAAGAAGAAGTTCTCTGAGGAACATGTGCCTATCCTGGATTA
CTACCATCGGTTCAGCACGTCTTTATTCTGGGAAATACAAGATATCGTCCCATTT
GGTAACCATCCTCTATTTCGGTATCTCCTCGGCTGGCTGATGCCCCCCAAAGTGG
CCCTCCTCAAGCTGACCCAAACCCAGACCATTAAGAACCTCTATGATAAACATCA
CGTAGTACAGGACTACTTGGTTCCTATCGAGGAGCTTAGATCATGTGTACACTAC
TTTCATGACAATATTCAGATTTACCCCCTCTGGATTTGCCCTTTCCTGCTGAAAGA
CCTGCCTGGTTTGGTTCATCCTGCTAAGGCACAGGATGGTATGTATCTAGACTTG
GGACTGTATGGAGAGCCCAAAGCTAAAGACTACCACAGCAAGAACACAATCACC
GCCTTGGAAAGCTATCTGGGAAAAATCAGAGGGTTCCAAATGCTAGGCGCGGGT
GTCTACCAATCATATTCCGAGTTTCGACAGAACTATGACCATAGCTTATACGACA
GAGTACGAGCCAGACTGGGCTGTGAAAAGGGTTTTCCCGTCATCTATGATAAGGT
CAATAGAGTTGCCCGGGACTGA (SEQ ID NO: 39)
#A-116 DC3Osc03 29905893-29905495 (RC) incomplete: ACP C-7 cholesterol
desaturase #116
TTTTCCACTGTAAGGAATATTCACGCAGCGCCCATCATGTCCACTGAACTGCCAT
TGATGAAACGGACACTCAATACAATCCCCTCTCACGAATCCTCCGATGGCAATGT
TAGCTCCCAAATGAGGACAGTACGCATCCAGCACATGCACTTCTCCCCTCTCCGA
TCGGAACACAGCAAAGTTTTCCCCGAGTGCGGAAACATATTTGGCCTGTTTGGGT
TTCAGCTCCGAGGATTCTAGCAGTGCAAACCACCCGTTTGGATACACGGGCGGG
AGGTTGCCTATTTTGCGCGCCTTGCGAATGTCGTTGATGAGCTGTTTTTTCGTTTT
GCCGTACTTGTTGTAGCCAGACCCTCGTCCGTCCAAGTAACTGTAGCCAACTTCA
CTCAGATCCTGTAA (SEQ ID NO: 40)
#160 Dcitr07g02580.1.1 ACP RAC1
ATGCAAGCCATAAAGTGTGTGGTTGTAGGGGATGGTGCCGTGGGTAAGACCTGT
CTGCTGATCAGTTACACAACCAATGCATTCCCCGGCGAGTACATTCCCACTGTGT
TCGATAACTATTCCGCCAATGTCATGGTCGATGGTAAGCCGATCAACCTGGGCCT
CTGGGATACGGCAGGACAGGAAGACTACGATCGACTCCGGCCCCTCTCCTATCCT
CAGACTGACGTGTTTCAAATCTGTTTCTCCCTGGTGAACCCGGCCTCATTCGAGA
ACGTGCGCGCCAAGTGGTACCCTGAGGTCCGTCACCATTGTCCAAGCACTCCCAT
CATCCTGGTGGGAACTAAACTGGATCTACGCGACGACAAGGAAACCATTGAGAA
ACTCAAGGAGAAGAAACTGGCACCTATCACATATCCCCAGGGTCTATCAATGGC
GAAAGAGATAGGCGCCGTGAAGTATCTCGAGTGCAGCGCCCTCACACAGAAAGG
TCTGAAAACTGTGTTCGATGAGGCCATCCGAGCCGTACTCTGTCCTGTGCCTACT
GTGCCCAAAAAGAAACGCTGCGTGCTCCTGTAA (SEQ ID NO: 41)
#A-165 Dcitr12g05570.1.1 ACP Crc
ATGGCTTCTTCGGGAATATGGAGTCTGAAAGGTACCCCAGGTATCTTGGAGGTCT
ATAATTATGTAGACTCCACATTAGAAGACAAGGGAGATGAAGGCAGCACCACCA
AGAGAGCAGAGGTTGCCTCCAAACTTCTAGAAACACTTGAGAAGTTCAACTACG
ATGAAACAACAGAACCCGCAGATTCTTACATCTCCGACTGGCTCGGTGGCGAAG
AGAAGGTCATCGACCTTCCCATCTACGAGGACTTCCCACTGTCCCAACCTTCCAT
CGTTCAACCAGGTAGCGCTACCGTCGTTCAACCAAATAGCGCTAGCATCGTTCCT
CAAGGTAGCGCCAGTGTCGCGTTCCGTCAAGGTCACGCGGGCTTCGCTCAAGGTC
ACGGCCAAGGTCATGTGAAGGTCGGCTACGAGGCGAGCCCCGTGTGGCAGGCCG
ATGAGTACTCGAGCCTGTATTCGCCTCCGGCCGCGCAATGGCCGGTGTATAACGA
GATGGTGGCCGCGGTGTCGAGTCAGCTGACCCCGCCCCACAGTCCCAACATGTA
CGAGCTGAGCCCCAAACACGCGGCCGAGTTTAAAGATCTGGCCGATGACTTGAT
GAAACCTGCGGCCGGCAAACTTCCGCCCTACCCGTCCGATATCGGCCTCCAGGCC
ACCAGCCCCTGTCTCCTCCCCCCTCAACCTGACTACGAGGAGAATCTGAAGGACG
GAGGTCAGCTCATTCTGTCTCTCCTGGCCGAGATGAATCCCAAGGACATCAGTGA
GCTGGTCCAAGCCAACGAGCTTATTCAAGACCAAGGTCAAACAAGCGAGCCCTA
CACCGTCCCCGATCTGAGCGCTCCAGCGAGCAGCGGCGCAAACATGAACTACAG
CGAGGTCATCCTTAGTCCGGACCACTCGTGTTCTTCCGACTCCAACTTCGACTAC
ACCTCGGACACCTCTTCCGACCCGGACTATATTCCGTCGGCGCGTGTCGGGTTCC
CTCGGAAACCGAGAGCTCCCGAAGTAGCTTCGGGGAAGATCGGCTCCGCTCGGA
AGGAAACGAAACGGGCCAAGCCGTATGCGCGTAAGGCGGCGCTACCTATTGAAG
ACAAGCGCCTGCGCAAGAAGGAGCAGAATAAGAACGCGGCGACGCGTTATAGG
ATTAAGAAGAAGGCGGAGATTGAGGAGGTTCTGGGAGAGGAGAAAGAGCTGTT
GGAGAAGAACGCCCAGCTGCAGAAGAGTGTGGAAGATCTGAGCCGTGAGATCA
AGTTTATGAAGAAATTCATGCGGGACTTCTTCAAGAAACAGGGCGTGCTCAAAT
GA (SEQ ID NO: 42)
#A-178 Dcitr07g07740.1.1 Chitinase 7|IPR001223,PF00704,GO:0005975
ATGATATTGTGGAAAGATGGAGGAGCAGTCAGTAAAATATCGTTCCTTCTGTTAG
CCATTGTATTCTTAGCTTCCTCTACATACAGCACACAAACCGCCAGGAGGCGACT
GCGACGGCCCTCCATCAAGTCCAAACCGTCCGAAGACTCCGGGGACCAAGATGT
CTCTGCCAGCATCAACAGGTTCAAGCTCAAGAACAGAACGGCCGCCTCTTCGGC
CAACAGCGCCGTGATCGCTGCCGGAAGCAGCAAGTCCAGTGTAAAAGCCAGCAA
AGATGACCCCAAGTATAAGGTCGTCTGTTACTACACGAACTGGTCCCAGTACCGG
ACCAAGATCGGCAAATTCCAGCCAGAAGACATCGAGCCCGACCTTTGCACGCAT
ATCATTTTCGCTTTCGGATGGCTAAAGAAAGGCAAGCTGTCCAGTTTCGAATCGA
ACGACGAGACCAAAGACGGCAAGGTGGGACTGTACGAGAGAATCGAGCAGCTG
AAGAAGGCCAACCCCAAACTGAAGACTCTGCTAGCCATAGGAGGATGGTCGTTC
GGAACCCAGAAGTTCAAGGAGATGTCCAAGAGCCGTTACTCTCGTCAGACGTTC
ATCTACTCGGCCGTTCCCTTCCTGAGACAGAGAAACTTTGACGGTCTGGATATCG
ATTGGGAATACCCTGCCGGCACTGACGACAAGAAAAACTATGTGCTCCTATTGA
AAGAACTGAGAGAAGCCTTTGAAGCCGAAGCCCAAGAAGTCAAAAAGCCCCGC
CTCCTTCTGACAGCTGCTGTACCTGTGGGTCCTGACAATATCAAAGGTGGATATG
ATGTGCCCGCAGTCGCAGGCTACCTGGACTTCATCAACTTGATGGCGTACGACTT
CCACGGCAAGTGGGAAAGCCAGGTCGGCCACAACGCCCCCCTCCGCTCGCCCAG
CTCTGACTCCGAGTGGCGTAAGCAGCTCAGCGTGGAGAACGCCGCCAACATGTG
GGTCCGTCTGGGTGCTCCCAAGGAGAAGCTGATCATCGGTATGCCCACTTATGGA
CGGACGTTTACCCTGACCAATCCGGCGGTGTTCAAGGTCAACAGTCCCGCGAGC
GGCGGAGGAAAAGCTGGAGAGTACACCAAGGAGAGTGGATTCCTCGCCTACTAC
GAAGTGTGTGAAATGTTGCGCAACGGTGCCGGTTACGTTTGGGACGACGAGATG
AAGGTCCCCTACCTGGTACACGGGGATCAATGGGTTGGCTTTGATGATGAGAAGT
CCATTCGGTACAAGATGAACTGGTTGAAGGACAATGGTTATGGAGGTGCCATGG
TATGGACTGTGGATATGGACGACTTCACCGGAACTGTTTGTGGAGGGGACGTGA
AATATCCGCTCATTGGAGCAATGAGAGAAGAGTTGAACGGCATCCCCCGAGCAA
ACAACGCCAAAGACGTAGACTGGGCCAAGGTAGCTGGCAATGTGGAAGTGGAG
ACCGTCACCAAACCAGCCCCCATCAAGATTGATGTGTCAGAACTGCTGTCGAAG
AAGAAAACGACTACCATTCAGAAACAGGAGACTAGTAACCTGATTGACACCAAA
GTTCGTCCGGCCCAGATCTTCTGTTACATGACCAGCTGGTCTCAGGCCAGACCCG
GCGCTGGCAAATTCGGTCCAGAGAACATCGACCCTAAACTGTGTACCCACGTGAT
CTACGCCTTTGGAACCCTGAAGGATCACAAGTTGACCTTGAACGAAGATAAGGA
GAAGGATGAGGCTGGCAAATTCGAAGCCCTGATGCAACTGAGAGAGAAGAACCC
TGACATCAAGATCCTGTTAGCCATCGGAGGATGGGCATTCGGCTCCACCCCATTC
AAAGAACTGACTGGCAATGTGTTCCGTATGAACCAGTTTGTGTACGAATCAATTG
AGTTCCTCCGAGAACACAAGTTCAACGGATTGGATGTCGACTGGGAATATCCAA
GAGGTGCAGACGACCGGGCCTCCTACGTGAACCTGCTCAAGGAACTGCGGCTAG
CATTCGAAGGAGAGGCCAAGACCTCTGGCGAGCCTCGTCTCCTCCTGACAGCGG
CCGTACCTGCCAGTTTTGAGGCCATTGCGGCCGGATATGATGTGCCCGAGATTTC
CAAATACCTGGACTTCATCAATGTGATGACCTACGATTTCCACGGCCAGTGGGAG
AGACAGGTCGGCCACAATAGTCCGCTGAAACCACTTGAAGGTGCCACCAGTTAC
CAGAAGAAGCTCACTGTCGAGTACAGTGCCAAGGAATGGGTTAAGCAAGGTGCC
CCCAAGGAAAAGCTGATGATTGGTATGCCTACGTACGGGCGTAGTTTCACTTTGG
TCGATCCGACTAAGTTCGACATTGGAGCTCCAGCCAGTGGCGGTGGGGAGCCTG
GAAAATACACGGCCGAAGCTGGATTTATGTCCTACTATGAAGTGTGCGATTTCTT
GAAGAAGGACAACACGACCCTGGTCTGGGACAATGAGCAGCAAGTTCCGTTCGC
CTACAAAGACGATCAGTGGGTCGGATTTGATGACGAGAGAAGTTTGAAAATGAA
AATGGACTGGCTCAAGGAAGATGGCTACGGAGGCATCATGATCTGGAGTGTGGA
TATGGATGACTTCCGAGGACAATGCGGCTCGGGCAAATACCCTCTGATCAGCACC
ATGAATACTGAGCTGGAGGACTACACGGTGGCTCTTGAATACGACGGACCCTAT
GAAAGTTTCAGCCCCAGCGGAAAGTATACCACCAAAGAACCCAACGTAGTTTCC
TGTGAGGAGGAAGACGGCCACATTTCCTACCACCCGGACAAGGCGGACTGCACC
CACTACTACATGTGCGAGGGCGAGAGGAAGCACCACATGCCCTGTCCCAGCAAT
CTGGTGTTCAACCCGAAGGAGAACGTCTGTGATTGGCCCGAGAACGTCGAAACC
TGTTTGCATCACACCCAGGCGCCTCCACCGTCCAAATAA (SEQ ID NO: 43)
#A-181 Dcitr04g09970.1.1 chitin synthase-RA|PF03142
ATGAGTACCCTCAGACAACTTAACATGCAGGATTCGGACCCTGATGACTTCTCAG
ACGATGAAAGTTCACCCCTGTCTCCCAATGATATCTATGGCGGAAGCACCCGTAC
AGTTCCCGAAACGAAAGCATGGGACGTGTTCCGAGACCTCCCGCCTCGACAGGA
CAGTGGCTCGATGGCATCAAAAGCTTGTCTCGAATTCTTCATCAAGGCGCTCAAA
GTGTTTGTGTATCTGTTTACGTTCATAGTGGTTCTGTTGTCCGGGGTAGTCTCTAA
GACTGCCGTGTTCTTCATGACGTCACAACTCAGAGCCCCGGACAAGCACATCCCC
TATTGTAACAAGGAGTTTGGTCGAGACAAGCAGTTCGCGGTGTATCTCCCAATCG
AGGAGCGTATCGCCTGGTTCTGGTGTCTCATAGCCGCCTTCATCGTTCCCGAACT
AGGCACGGCTATCCGGTCCATCCGAATCTGCTACTTCAAGTCCTGGAAGAAGCCG
CCCCTGCACGATTTTATCTTCGTCACCTCCATGGAGCTGTTGCATACCCTGGGACT
GGCCTGTCTGGTGTTTGTAGTACTGCCCAACATCGATGTGGTGAAAGGAGCTATG
CTTACCAACTGTTTGTGTTTTCTGCCTGCCTTCCTCACCCTGATATCTCGGAACAC
CAGGGGCTGTACGAAGGGTTCAGAGAGGTCCGAAGTATACCTGAAGGCCATGAT
GGACATGATAGCTGTATCTGCCCAGGCTACGGGGGCCCTCTTGTGGCCCCTGTTG
GAGGGGCACAATGACCCCTGGCTGTGGCTGATACCGCCGGCCCTGTTCTGTGTGT
CCTGCGGTTATTGGGAGAACTACACGAGCAAACATTCAATATTCGGTTTCATGAA
GTCAGCATGGCGGGTTAAGGAGAGACTGAAGCGTACCCGATACTTCACCTATGC
CTTCGTGTCATTCCTCAAGATCCTGGTATTCCTGTGTTCTGCTCTCGTATTCATGTC
ATTCCGCGGAGAAAGCGTTACGGAATACTTCTCCAAGTTCCACGACTCCTTCTCA
CAGCACAAGATTCGAATTTATGAGGTGAAAGCTTCGGCATTTGGCACTTCTATCC
CAGACTTGGCTGATCCTAATCTTACAGGCGATTACAGGGATGTAGATTCCGAGGA
TAATTTCGCCATGAAGATCCTGCTTTTCCAGATCTTTGCTGCATATCTGTGCTACA
TTTTCGGCAAGTACGCGTGTAAGATCATGATCCAGGGTTTCAGCTACGCGTTCCC
GGTCAACCTGACCATTCCAGTCACCATTTCCATTCTGATCACAATGTGTGGACTG
AGGAATGATGATCCGTGCTTCTTCCAGAACTCCATCCCGGACTATTTGTTCTTCGT
GTCCCCCTCTATCTATTTCCTCGACGATTTCATATTGAAACAGCACGCCTGGGTTT
GGTTGCTATGGTTACTGAGTCAGACTTGGATTACGCTTCACATTTGGACCCCCAA
GTGTGAGAGGTTAGCCACCACTGAGAAGTTATTCGTACGTCCTATGTATGACGCA
CTGCTCATTGATCAGTCCATGAGTTTAAATAGGAGATGCGATGATGAGAAAGAC
GTCAAGACAGAGGAACTCGTAGATCGAGAGAAAGATCCAGACGAGTACTACGA
GACGATTTCCGTTCACACGGACGCCTCGTCCACAACGCCCAAGACCGTGAAGAA
ATCCGACTCCATTACGCGCATCTACGCATGCGCAACCATGTGGCACGAAACGAA
AGACGAGATGATGGAGCTGGTCAAGTCTATTCTGCGTTTGGATGAGGACCAGTG
CGCACGGCGCGTTGCGCAGAAATATCTGCGCGTGGTCGATCCGGATTATTATGAG
TATGAAACGCACATCTTTTTCGACGACGCGTTCGAAATTTCCGACGTGAATGACG
ATTGGTCGCAAGTGAATCGGTTCGTCAAGCTATTCGTATCCGTCATTGACGATGC
GGCGGCGCATGTGCACGAGACCAACATTCGCATCCGGCCCCCTACCAAGTATCC
GACACCGTATGGGGGTCAGCTCGTGTGGACTCTGCCTGGGAAGACCAAGATGAT
TGTGCACCTGAAGGACAAGGCCAAGATCCGGCATAGGAAACGATGGAGTCAGGT
GATGTACATGTACTACCTGCTGGGTCACCGCCTCATGGAGTTACCCCTACCGGTA
GACAGGAAGGAGGTTATGGCCGAGAACACTTACCTACTCACCTTAGACGGAGAT
GTGGACTTTCAACCGGACGCTGTACGACTACTGGTAGATCTGATGAAGAAGAAT
AGAAACCTGGGTGCCGCGTGTGGACGTATTCATCCTGTGGGAGGAGGTCCAATG
GCTTGGTACCAGAAGTTCGAATACGCCATCGGTCATTGGCTGCAGAAAGCTACCG
AGCATATGATTGGCTGTGTACTTTGTAGTCCTGGATGTTTCTCTCTGTTCAGAGCC
AAAGCTCTCATGGATGATAATGTAATGAGACGCTACACTACCAAATCAGCTGAG
GCCAGACATTACGTGCAGTACGATCAAGGTGAGGATCGTTGGCTCTGTACACTCC
TACTACAGCGAGGCTATCGAGTAGAATATTCCGCCGCCAGTGACGCTTACACCAG
ATGTCCTGAGAGTTTCAATGAGTTCTACAATCAGCGGCGGCGATGGGCGCCCTCT
ACTATGGCTAACATTATGGACCTGCTCATGGACTATAAGAGAACGATCAAGATC
AACGATAACATTTCCATGCCCTACATTTGGTACCAGGTGATGTTGATGGGAGGTA
CCATCCTAGGTCCGGGCACTATATTCCTCATGTTGGTGGGTGCCTGTGTGGCCGT
ATTCCACATTGATAACTGGACCAGCTTCTACTACAACATTACGCCCATTCTCTTCT
TCATGTTTGTCTGCTTCTGCTGCAAGTCCAACTTACAGCTCCTCCTGGCTCAAATC
CTATCGACAGCGTACGCCCTCCTCATGATGGCCGTCATAGTAGGTACCGCCCTAC
AACTCGGCGAGGACGGTATTGGGTCCCCGTCCGCTATTTTCCTCATTGTCATGTTG
GGGTCGTTCATCATAGCCGCGTTACTCCACCCGCAGGAGTTCTTCTGTATTGTATT
CTTCTCTATATATCTCCTATCGGTGCCTTCCATGTACTTGCTCCTGATTCTGTACTC
GCTTATCAATCTGAACGTGGTATCCTGGGGAACGCGTGAGGTGGCTGTTAAGAA
AACCAAGAAGGAACTCGAGGAAGAGAAGAAGGCCGCCCTGTTGGCCAAAAAGA
AAGCCAAGAACCCGCTGCTCGGATTTCTGCAGAACTACTCACCTGAGGCTGATGG
TGATGAAGAAGGTAGTCTGGAGCTATCGTTTGCCGGTCTGTTCAGATGTATGTTC
TGTACGTATCCCAAGCCAGTGGATGAGAAACAACAACTGCTTCGCATAGCGGAC
AGCTTGAGTTCGTTGGAGAAACGCTTGGACCATATTGAACGAGTGGTAGACCCG
AACATGCCCACCCCGCACGGACACCGCCGACGTACTACCTCAGCCTCCAGTAGC
GCTCACCATGCCATGGGTCTGGGTTCGGTTCCCGAGAATAAGAGCGAAGATGCG
TCCAGTAATGAGGAAGATGAAGACAGCGATACAGTGTCCACAGCGGAACCCAAA
ATCGAACGCAACGACGACATTAACCCCTACTGGATCGAAGACAAGGAACTGAAG
AAGGGACCTGTTGCTTTCATTTCACCCAACGAGAGAGCATTCTGGGTTGATTTGA
TAGACAAGTATCTCTACCCCATTGATAACGACAAGGCTGAGCAGAGTCGAATAG
CGAGCGATTTGAAGGACTTACGGACCAGTCCGTGTTTAGTTTCTTCATGGTGAAT
GCCCTGTTTGTCGTGATCGTGTTTTTGCTTCAACTCAATAAAGATAAATTGCACAT
CCGCTGGCCATTCGGGGTTAAAACGAACATTACGTTCAATGAGTCGACCCAGGA
GGTTATCATCACCAAGGAATATCTACAACTGGAACCCATCGGTTTGGTGTTTGTG
TTCTTCTTCGCCTTGATCCTGGTGATTCAGTTTACCGCCATGTTGTTCCACAGATT
TGGCACCATCTCGCACATTTTGGCCTCGACGGAGCTTAATCTATGTTGCAACAAG
AAGACTGAAGAGCTCTCCCAGGACGGTCTCCTAGACAAGCAAGCGGTGGACATA
GTCCGCCACTTGCAGCGACTACGAGGCATTAACGGGGAGTACGAAAACGACAGT
GGCTCCTCCGGCGACCGAGTGGGCAGAAGACGAACTATATACAATCTCGAGAAG
CAGAAGAACAAAACGCGAACCATTGGCACTCTGGACGTGGCATTCCGAAAACGG
TTCCTCCAGATGAAGATGGAGGATGCTGTAGAGACAGCGGGTACCCCAGTGCTC
GGGCGTAAGTTAACCATGAGGCGAGAAATCAGAGAAGCTCTGGAAGTGAGGAG
AAGATCCTTACAAGCCGAGAGAAGGAAGTCCAACATGCAAACATTGGGGGCTCA
TAATGATATTTATGGCCGAACTCAAAGGATCTCAAACGCGGGATCTCACGTGAA
GGACATTTTCGACGGCCATCAGAACCCTGCATACGAGCCTGATCTGGCCTCGGAT
TCGGACAGGCCTTTGCCGGCCACTATGGAAAACAGGGCCGTCAATTGGGCCGAG
CTTGAAAATCAGCGGTCAAATAGTCACATTTAA (SEQ ID NO: 44)
#A-184 Dcitr06g10380.1.1 Endochitinase/Chitinase 5|IPR001223,PF00704,GO:0005975
ATGGCGTTTCCTTTTTACGGAAGTATTGCACTTTTGGCGAGTCTCATTTTCATCTC
GATTTCTCCTACGTCTGGGGATGACTCAGACGTCCCGGCCAGGATATCCTGTTAT
TTCAGTAACTGGGCAATCTACAGACCCGGAATTGGCAAGTACACCATAGATGAC
ATTCCCGCGGGTATGTGCACTCATTTGATCTACGCGTTCGTGGGCATCAGCAACA
GTACGTGGACTCTGGAGGTGTTGGATCCTGAGGTTGATGTGAAGGAGCATGGGTT
CCGGAATTTCACCGCTCTGAGGAAGACACATCCGGGATTGAAAGTGGAGGTGGC
AGTAGGCGGCTGGGGCGAAGGGGGAGAGAAATATTCCGCCATGGTGGCCGACA
AAACGAAACGAGCCACGTTTATTAGAAGCATTGTCGCTTTCATGAAGAAATACA
ACCTGGATGGGTTTGATTTGGACTGGGAGTATCCCGCCGCTTCGGACAGAGGTGG
CAGCTTCAGCGACAAGAACAACTTCTTCTTCTTTGTGGAAGAGCTGAGGACGGCG
TTCAACAAGTTTGGGAAGCACTGGGAAATTACCATGGCCGTTCCGATGGCCAAGT
TTCGACTCAATGAAGGCTATCATGTGCCAGACCTTTGCGAACTTGTGGACGCTAT
TCACGTCATGTCTTATGACCTCCGAGGCAACTGGGCAGGATTTGCCGACGTCCAT
AGTCCACTTTACAAGAGACCTTCGGATCAGTGGGCGTACGAAACACTCAATGTG
AACGACGGTCTTCAGTTGTGGGTAGACAAGGGATGTTCTCCTCGCAAGCTAGTAG
TGGGGGTGCCATTCTATGGTCGCTCGTTCACTTTGAGTTCCGGAAACAACAACTA
CAACATTGGCACCTACATTAATAAGGAGGCTGGAGGTGGAACACCAGGTCCCTA
TACAAATGCCTCTGGATTCATCTCGTATTATGAGATTTGTTCCATGGTTCAGGAA
GATAAATCCTGGGTTCAGAAGTGGGATGATATTGGAAAATGCCCTTACACATATC
AAGGCACCCAGTGGATCGGGTACGAAGATCCAAAGAGTATCCAAATTAAGATGG
ACTGGATCAAATCTAAGGGGTACGCTGGAGCCATGACTTGGGCTATAGATATGG
ATGATTTCCAAGGACTTTGTGGACCCAAGAATGCCCTGATGAGCATTTTGTACGA
GAACATGAAAGACTACAGGGTTCCTGTGCCTCATGTTTCTATTACACCACGGCCT
GAATGGGACAGACCTCCCAGCACTACCCCCGAGGACATAGATTATGCTCCCACC
TTTACGTTCACCACAAAACCCGACCTTCCATCGACCCCAACCACAACAGCCACCA
CCGAATCATCAACTCCAGAAGTCACCAAACCTGTGGAACCACCAACCACACCCA
AGTACAAACCCAACAAGCGACCACGACCTTCCATCACGGCCAAACCTCCGCGCA
CAACTACGGGCAAACCTGAGACGAATCGACCTGACAGTGCTGAATCAAATGAGA
CGAATGGACCAGACAGTTCTGAGACGAATCAGCCAGGGAGTTCTGAGACGAATC
GACCCAAACGTCCAGTGAAAAGGCCACTTCGCCCAACAGTCAACCGACCTGATC
GCCCACCAGTAAACCGACCTAATCGACCTGAGACTGTTGAAGCAACCAGTCAAG
AAGTTCTGGAGCCAAGTCCTGAGGTCACTGAGGAAAACCGGCCGGAAGGTAACC
TACCCGAAGAGACTGAAGTGGATTGTTCTAATGATGGATTCCAGTCGCACGCTCT
GTGCAATAAGTACTACCAGTGCGCGTTCGGCAAACCAATCGAATTCACGTGTCGA
CCTGGTACCTACTTCAATCGGAAAATGAGCGTCTGTGATTGGCCAGAGAAAGTCG
ATGTTACCCGATGTAAAATGGTCAAACCCCTGAAGACTGTCGAATATGAGGATG
AGAACTACTTCCTGTGA (SEQ ID NO: 45)
#A-190 Dcitr01g18710.1.1 WAVE
ATGCCGCTACCCAAGCGAGTGATAGAGCCAGTGCATGTGTCCCGGGGTACAATA
CCTGAGGAGCTGTCTGCCAGCCTCCCGTCGGAGCTTGAGGCAGCCACCAATGGA
ACTCTGGCCAACACGGTTCGACAGCTGTCCAGTCTGAGTCGGCATGCGGAGGAC
ATGTTTGGTGAGCTTACCCGTGAGGCGCATAACATGGCCATGAGGGCCAACTCCC
TCCAGGTTAGATTGGACCGGTTGGCAGTCAAAGTGACCCAACTGGATAGTACCAT
CGAAGAAGTGTCGCTGCAAGACATTCACCTGAAGAAGGCATTCAAATCGGCCAT
CGTGTTTGATCAGGAGGTTGTATCTCGCAGTACCATGCCCACAGCTATGCTCGAG
ACCTACAAGCAATGTGATAAGCCACCGCCACTCGACAAGTTAGATCCGTACAGA
GAGGATGGCAAGTTTGGTTTGAAGTTCTACACAGATCCTGGTTACTTCTTCGACC
TATGGAGACAGGAGATGCTCAAGGATACAGAAAGGATGATGCATGATCGCGGGA
AAAAGCCACACCGACCCCGCACGACAGAGAGCGGCGGCGGGCGGCGACAGAAG
CGCGTCCGGGCGCTTCACAACACGCGGGAGCGCCAACGTCAGCTCGCTATGAGT
CACGGCGAGTACATCATGCCTCACCAGGCGATGTATCCCCCGCCCGGCTACTCCG
AGGAAGATATGATGATGGACGGGGGCAGACCGTCGCGGCCCGACAGCATTGACC
TGTCGGGCCGACACTATGTGGACGGCGCGGGCATGCCCGACGGCGGCATATACA
GACCCGGTCTGCAGCAAGGCTACATGAACTACGACCAGGTGATCTACCAGCATG
GCTCCCAGGGCGGTCACTACGGTACTGGTATGCTTCACTACGATATGAACGCTAT
GGGCAACAACGTGAACAATAGTAATCTAGAAACCTACACATCCCCTGGTGGCAC
TCCGAACCGAACCAAACCACGACCTTCCCAGCCTCCCCCTGCCCCGCCCAGCACT
ACCAATTCCAATAATAGTACCCCCACCACGGTATCCGCGAGCAACACTCCCACCC
GGGCCCGCAGCATGTCTGCCGGCCGCGACATTCTCNNNNNNNNNNNNNNNNNNN
NNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNN
NNNNNNNNNNNNNNNNNNNTGCCCCCACTGATGAACGGAGACCTGCACCTGCC
CAACAGTACCAAGTCCCCTCTCAAACAGGGGTCGACTACTTCGCCGCCAAAGCC
CAGTAACATAGACGTACGCAAACCCGTGTATGATCCTAGGAGTGATCTGCTCAA
AGCTATACGAGACGGCATCACGCTACGCAAGGTAGAGCGCGTAGAGCAGAAGG
AAGGCGAGCGGCAGAAAGCGCTGCATGACGTAGCATCCATTCTGGCGCGCCGAG
TAGCGGTCGAATTCTCCGACTCTGACTCCGCCAGTGAGAGTGAATACGATAGCGA
CGGGTGGCAAGAACAGGAGACCAGTGCTTAG (SEQ ID NO: 46)
#A-192 Dcitr03g10670.1.1 Brain-specific angiogenesis inhibitor 1-associated protein 2-
like protein 1
ATGGAAACCGAAGAAATAACCAAACTGGTTGATTCCATTTACAAGAATATCAGC
GACAAGTTCAATCCTGGAGCTCGTCAGATGATCAGTGCTGGGAAAGCTTATCTCA
AAGCTTTACATGGAGCCGCAGCTGCCTCTAAACTTTACATAGAAGCTGTTTCCAA
ATTGGGCAAAAATTCCCAACAAGCACTATGGGGATCTTCAGCAGATGTCGGTAC
CTCACTCCTTCAAATTGTAGAAGTTTGGAACGAAGTCCAGTCTCAACAAATGAAC
ATTTTGAAAGCATTCTACGTGGACATGATCGTGCCCCTGGAGACAAACCTGGAGA
AGGACACCAAAGTGGTGCAAGCCGAGCAGAAGCGGTTCTTACAGCAGCACAAGA
TCCTCTCCGAATCTTACAGTAAGGCGGCATCCAACATGAAGAAACAGAGAAAGA
AACACAAGAGCAACTCCAAAGCCAGCAACACAGTCAGCAACAAGGAGATCAAA
ACAATGCAGATTCTAGAAGAAGAAAAGTCAAAGCTGGATGCGTTCTGTGAACAA
GGATTGAAAAATGCTATGACACAGGAACGGCGTAGATACGGGTTCGTTCTGGAA
CGGCTCTCTAGTCTGGCTAAACACTACCTGGCGTACCACTCGGCTGGCAACACCC
TGTATGAGAAGAACATAGATGAATGGATTGAGGTGACCAAGACCAGGGAAATGC
TACCGGACAGTGTGGAGGCAATCTGTGCCAAACGATTATGGCAAGTCAACTATT
GGCCGGATGAAGGCGAGGATGATCTAACCTCCGTGACGTCACAGTTGAGGAAGT
GCAAAAGTATGGATGCGGCTTCTCTCATCACAGACATAGCCCGACCTCTGCCCCG
CGCCCGCTCTGATTGCAACCTGTCTACACCACCACAGAGCCCCATGTCTAGAAAT
GATTCCAGACCTCAGTCATCCTGGGAAGCTCCTGTTGCCAAAGCTTTATACGCGT
ACATGTCATCTGGAGACAATCAGCTTAACTTCATGGAGGGAGATATGATTGCTCT
TACAGGTGATAGAAATAAAGGCTGGCAATACGGAGAAAACCTGAGAACCCAAC
AGAGTGGCTGGTTCCCATGTGCTTATGCTGAGCCTATGCTGGAAGAACAACACGT
GACGCCATCTCACAGAAGATCAGCTTCGGGTAAAAGACCTACCACTTCCCCCCCT
CCCCCTCCTGTAACCAAGTTTGGTGATAGTTTGAGATACCAGCGTCGAGTCGGGA
ATTCTACGTTGAGTATTGACAGAGGCTCCCCACCTCAGATAGCTCCGCCACTGGT
TCCTTCCCTTCACAGTAGCAACGACAGTGGCTTTGGCAACGACCCGCCTGTGGAC
TACTCCGATGATGATTGTAACAGGAACTCTTCAAGTACATTGAAGAAGCCTTTAT
CCAAACAAATGTCACTTCAACCAGAAAGTGATGCGCTCAAAGACAAAGGTGTTG
TAAAGCGAACCAAATCCTCTCTGTGGAAATTCCGCAAGAATGACGATGTCCTTGA
GGGTATGAGTCTGTGGAGACATCGGTCTCTAGTGGATGTCACTAAGGATGAGAA
GACATTCACCTATGTGAAAGATATCAACATTGACTATGACGAAGATAAAGAAAG
CACTCTGGTCAATAACGATGATGAATCCTGTATTGTGGTCACGGACTTCAAAGTG
AGGAAAGACGTCAAAGGAAACGACTTCAGCCTACGCTCAAATAAGAAACCTGAT
CCTGACAATGAAATGAAGAGGAAATCCATCAGCAGTAATATTTATGAAATAGAG
AAACCTCTCGTGAAAAGAGAACAAGTGAGACGATATTATCGCGATTATACAACA
GACACCGAGGACACCAATGTCATGAAGGTCAACGAAGATTTACGACGAAAAAAC
CGCGAAAGTTTCATCGAAAAAGATATGTACGACATGAAACTGAATGTCAACTTG
GATAAGAGATCCAAGAAAGACATACCGACGGGCACCAAACAAAACAATGAACA
GATCAAAAAGGACAAAGCATCCACTCTGCAACGTGAAACACCAGATCGGAAACT
AGTTGAAGATCAAAAGAGAATTTCCAGAGAAATCGAACGCATTCGTAACGGAGA
CTTTGTTCAAAAAACCAATAACACCATGAAAGATGAAGACCACAGGAAAAGTAA
CGGCTACAAAATGATGAACGGGAACGCAAATCATAGCATGGATGACGTACGGAG
AAAGAATAGAGAATACAGTGTAGAGAGAGGAAGTTATGATAAACTGAACAATAT
GTATGACGAGGAGGAGAGAGACAGACGGAACTATACACTTGAAAAAGAAAGTG
ATATAAAGCAAAAGTGGAAAGAAAATTACGTCATGATGGGATACAATGAAAGG
AATGACTTGAGTAAAAGGTACAGTGTAGCAGGAGATAGCAAGATCAAACCAGAA
TATATTGAGGACAACAGGAGGTACAACCCTATCAGCAGCAAGAAACTAAACAAC
AGGAAAATTGAGACAGAGAGTGAAGATGATTGGTATGACTCCTGGGATGAGAAA
TAG (SEQ ID NO: 47)

The above listed genes and sequences are in no way to be construed as limiting to the present disclosure. Any suitable gene may be the target of the synthetic RNAs and methods described herein. For example, any suitable gene found in a hemipteran (e.g. whitefly, psyllids) may be the target of the synthetic RNAs and methods described herein, in particular for use in methods of pest control such as inducing fatality and/or infertility in the desired organism.

In some aspects, provided herein are methods for modulating gene expression. In some embodiments, provided herein are methods for gene silencing. In some embodiments, methods for gene silencing comprise providing to an organism a synthetic RNA described herein. Any suitable synthetic RNA, including those described above, may be provided to the organism.

In some embodiments, the synthetic RNA is formulated as a liquid composition. Such a liquid composition may be applied to a food source for the organism. For example, a liquid composition may be sprayed onto crops, plants, etc. such that the organism ingests the synthetic RNA during it's normal feeding cycle. When ingested, the synthetic RNA may induce gene silencing within the organism. For example, the synthetic RNA may be designed such that silencing of the gene of interest is fatal for the organism. Alternatively, the synthetic RNA may be designed such that silencing of the gene of interest induces sterility in the source organism and the organism is unable to propagate (e.g. unable to produce offspring). The liquid composition may additionally comprise suitable excipients, stabilizers, etc. For example, the composition may additionally comprise stabilizers or degradation inhibitors to prevent degradation of the synthetic RNA.

Any suitable organism may be the target of gene editing (e.g. gene silencing) using the synthetic RNAs and methods described herein. In some embodiments, the organism is an insect. In some embodiments, the organism is a hemipteran. In some embodiments, the organism belongs to the hemipteran suborder auchenorrhyncha (e.g. cicadas, leafhoppers, treehoppers, planthoppers, froghoppers), coleorrhyncha (e.g. moss bugs), heteroptera (e.g. shield bugs, seeds bugs, assassin bugs, flower bugs, sweetpotato bugs, water bugs), or sternorrhyncha (e.g. psyllids, aphids, whiteflies, scale insects). In some embodiments, the organism is a whitefly. In some embodiments, the hemipteran insect is a psyllid. For example, in some embodiments the hemipteran organism is an Asian citrus psyllid (ACPP) or a potato or tomato psyllid (PoP).

Suitable synthetic RNAs and methods of use thereof are described in Mondal et al., Life Science Alliance (2020) vol. 3, issue 10, e202000731, the entire contents of which are incorporated herein by reference for all purposes.

### EXAMPLES

### Example 1

### Materials and Methods:

### Whitefly Colony Maintenance

Insects in this study came from the type B. tabaci Arizona B biotype (AZ-B) whitefly colony established in Brown laboratory in 1988 after its discovery on poinsettia plants in Tucson, Arizona (Vyas et al, 2017). For this study, AZ-B adult whiteflies were serially transferred to and reared on cotton (Gossypium hirsutum L. cv Deltapine 5415) plants at the 8-10 leaf stage.

### Phylogenetic Tree Construction

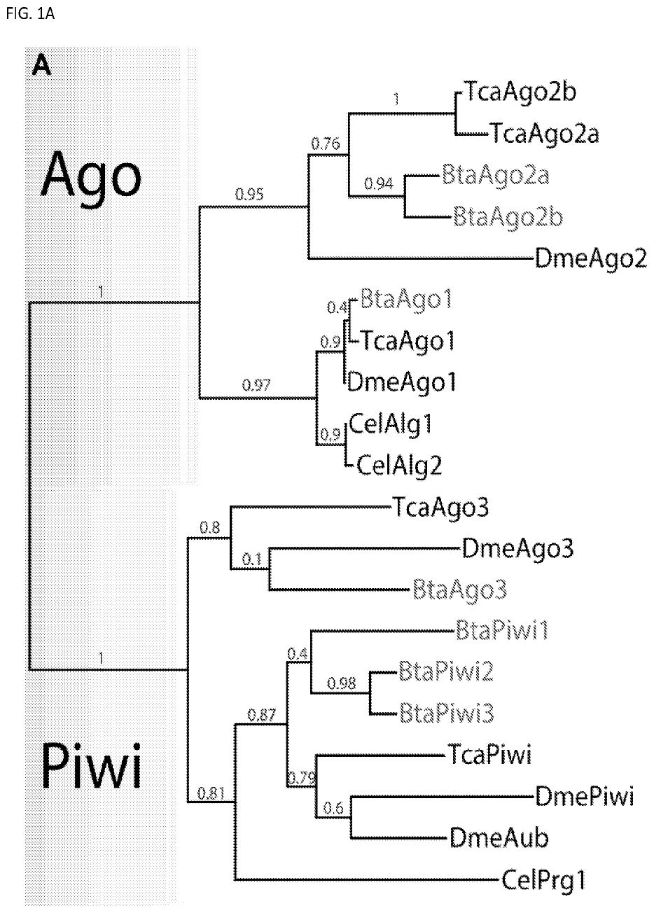

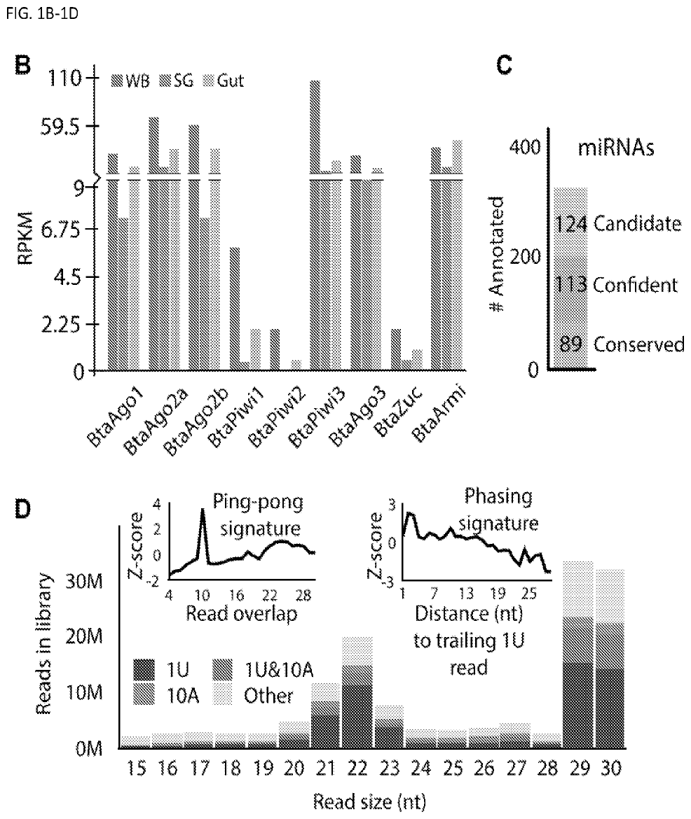

T. castaneum sequences of the argonaute proteins were downloaded from National Center for Biotechnology Information (NCBI) (EFA09197.2, Ago1; EFA11590.1, Ago2a; EFA04626.2, Ago2b; EFA02921.1, Ago3; and EFA07425.1, Piwi). Whitefly sequences were downloaded from B. tabaci MEMA1 genome database: ftp://www.whiteflygenomics. org/pub/whitefly and the argonaute sequences were curated using blast and protein domain search tools InterPro and ScanProsite. The final argonaute genes are Bta01840, BtAgo1; Bta00938, BtAgo2a; Bta12142, BtAgo2b; Bta04637, BtAgo3; Bta00007, BtPiwi1; Bta00198, BtPiwi2; and Bta08949, BtPiwi3. Annotated D. melanogaster and C. elegans sequences were also obtained from NCBI. The phylogenetic tree shown in FIG. 1A was reconstructed in http://www.phylogeny.fr suite. Multiple sequence alignment was carried out using MUSCLE, phylogenetic tree was constructed by maximum likelihood method, and the maximum likelihood tree was visualized by TreeDyn.

Cloning of Whitefly Sequences and In Vitro Transcription of ssRNA and dsRNA

AQP1 (KF377800.1) and AGLU1 (KF377803.1) sequences were cloned in pGEMT-easy vector. The cloned plasmids were used as templates for PCRs, which were used in ssRNA and dsRNA synthesis reactions. For creating the fusion constructs (adding piRNA/siRNA sequences to the gene of interest [GOI]: AQP1, AGLU1, and Luciferase sequences), the SOEing PCR method was followed as detailed below.

### Whitefly Fusion Constructs Synthesis and Cloning

PCR recipe for SOEing PCR

| | | |
|---|---|---|
| Phire Plant Direct PCR Master Mix | 25 | uL |
| Phire dilution buffer | 1.5 | ul |

| | |
|---|---|
| Fragment A (25 ng) | |
| Fragment B (25 ng) | |
| ddH₂O | make the reaction |
| | to 50 uL |

Thermocycler steps

| | | | | |
|---|---|---|---|---|
| 98° C. | 5 | min | | |
| 98° C. | 7 | sec | | |
| 53° C. | 10 | sec | | |
| 72° C. | 7 | sec | {close oversize brace} | 10 cycles |
| 72° C. | 5 | min | | |

After the first 10 cycles, end primers were added and the thermocycler was ran for another 25 cycles following these steps:

| | | | | |
|---|---|---|---|---|
| 98° C. | 5 | min | | |
| 98° C. | 7 | sec | | |
| 63° C. (Annealing Tm) | 10 | sec | {close oversize brace} | 25 cycles |
| 72° C. | 15 | sec | | |
| 72° C. | 5 | min | | |

Phire DNA polymerase doesn't create any ‘A’ overhang. For T-A cloning into pGEM-T easy vector, the ‘A’ nt was added to the final fusion products using Taq DNA polymerase.

### Sequences of the Constructs

Capital letter sequences are piRNA trigger, sandwiched lower case sequences are from gene of interest (AQP1, AGLU1, Luciferase)

AQP1-piRB-6
(SEQ ID NO: 8)
AGCAGCTTCTTGCCTCTGATTCCACGGTTTCTTCTTAAAGGGCCCCGACGACTGCT
GCGGGCCTTGATAAGGCGCGCTCCTGTTATTTGCCTCACGGAACGTCTTTTCCGC
GGCCATCATTGCGTCCATTGATCGGATCAAATCTTGCCTCATTGCATCCACGGCT
CGAGTATTCCTATCCGTATCCGCACGATTTAGATCAACTGCGTGTACCAAAGTCG
CTAGGGCGTTCTCATTGGCCTTCACCCGGGATTCTAAGGATGATTCCTGCCCCGT
ATAGTGATTTACGGCCAAAATAGCGCCCCTTCCTTTGCTGGTCGCGGCTACTGCT
AGCTTCGCATTtcgcacaatgccttggagccatctgtggagcaatcattctgaatgaaatcacgccaaaaacaggttacacg
gctgctggtaatctgggagtaacgacactgtctacaggagtttccgacctgcagggtgtggcgatagaagcactaatcacatttgtgct
gcttttagttgtccagtccgtctgcgatgggaagcggaccgacatcaaaggatctatcggcgttgcgataggattcgcaattgctTCC
GTCGAGTTAACTTTAGCCAAGCCCGCTAGTTTTCTCTTCGCTTGAACGTAATCCAA
CGGGTCCTCATTTTCTCCCTGCGTTCGCGCCGAGAATTTCGTGAGGGCATCCTCGT
CGCTGTCAAAGTATTGGATCAATTTCTTCTTTACTTCCTCAAAAGTCCTGCAGTTA
CCGAACGCTACCTCTTCATTGTCGTAGTACTGGATGGCACGTTTCGCTAAGTGAT
TTCTGAGTTGGTCCCGCTTTTCTTGATCCGAACATTTCTTATAGAAATTTTCAAAA
TCTTTTAGAAATTCTCTAACGTCGTAGTCAGCTTCTCCTTTGAATAGTTTTCTAAA
CGGCGGTGCCTTAATCGTCACCGTAGGT
AQP1-No_bias-14
(SEQ ID NO: 9)
TTGCGTTCCTGCTCCCTTTGCCCTTTACCGCGCTCAATTATCTCTATTAGAACCGG
AGATATTCGGTTTACAAAAATTTTTTGGGGCCCAGCCCCCCTTAATCCTTTCCCTA
TGGACTTCCTATATGGCCCCAGAGGTAGCCCCCGGGGGTTAGGCAAATAATCCCA
AAAAATTCCCAAATTCTAACGGAAATGTGGCACTACCGCCCCTACGTCACTCTGG
CTATGACGTAGTTGATtcgcacaatgccttggagccatctgtggagcaatcattctgaatgaaatcacgccaaaaacag
gttacacggctgctggtaatctgggagtaacgacactgtctacaggagtttccgacctgcagggtgtggcgatagaagcactaatcac
atttgtgctgcttttagttgtccagtccgtctgcgatgggaagcggaccgacatcaaaggatctatcggcgttgcgataggattcgcaatt
gctTTACGTGCCGTTACACCGGTTACCGACATCAGGTTCCTTCAAATCGGACACGG
GCGCCCCTCCCCGAGGGGATGCCAATGGGGGGAGGTCCCAGGCCGAAGCCTGAC
TTTCTACTACCTCCGGAGCTGTGCCCTTCTCTGCACGTCCCAGTTGAGCACTGGTG
GGCTGACCTCGGGGACAAGGTCGCCTTAACTTACCG
AGLU1-piRB-6
(SEQ ID NO: 10)
AGCAGCTTCTTGCCTCTGATTCCACGGTTTCTTCTTAAAGGGCCCCGACGACTGCT
GCGGGCCTTGATAAGGCGCGCTCCTGTTATTTGCCTCACGGAACGTCTTTTCCGC
GGCCATCATTGCGTCCATTGATCGGATCAAATCTTGCCTCATTGCATCCACGGCT
CGAGTATTCCTATCCGTATCCGCACGATTTAGATCAACTGCGTGTACCAAAGTCG
CTAGGGCGTTCTCATTGGCCTTCACCCGGGATTCTAAGGATGATTCCTGCCCCGT
ATAGTGATTTACGGCCAAAATAGCGCCCCTTCCTTTGCTGGTCGCGGCTACTGCT
AGCTTCGCATTCtgtccatccaaccctggattgccttttggtaatctttggcgggagagcgaccgctcacgtgcgtaataaag
aagaaattgaatggcatatgggctcctggtttcccctcaaactgataatagtccattgttctatcgagagttgtatatgcttctgtcattagta
cttttgttttcccctctttctttgtgtagaaatcgaagacctccctgaaccttgttatcaatctatatgtattcggttggtccatcgtccgcgacc
ggttgtaattccagtagttcgtcgggtcgagatcgggcgagagcaattcttggtctcgccattTCCGTCGAGTTAACTTT
AGCCAAGCCCGCTAGTTTTCTCTTCGCTTGAACGTAATCCAACGGGTCCTCATTTT
CTCCCTGCGTTCGCGCCGAGAATTTCGTGAGGGCATCCTCGTCGCTGTCAAAGTA
TTGGATCAATTTCTTCTTTACTTCCTCAAAAGTCCTGCAGTTACCGAACGCTACCT
CTTCATTGTCGTAGTACTGGATGGCACGTTTCGCTAAGTGATTTCTGAGTTGGTCC
CGCTTTTCTTGATCCGAACATTTCTTATAGAAATTTTCAAAATCTTTTAGAAATTC
TCTAACGTCGTAGTCAGCTTCTCCTTTGAATAGTTTTCTAAACGGCGGTGCCTTAA
TCGTCACCGTAGGT
AGLU1-No_bias-14
(SEQ ID NO: 11)
TTGCGTTCCTGCTCCCTTTGCCCTTTACCGCGCTCAATTATCTCTATTAGAACCGG
AGATATTCGGTTTACAAAAATTTTTTGGGGCCCAGCCCCCCTTAATCCTTTCCCTA
TGGACTTCCTATATGGCCCCAGAGGTAGCCCCCGGGGGTTAGGCAAATAATCCCA
AAAAATTCCCAAATTCTAACGGAAATGTGGCACTACCGCCCCTACGTCACTCTGG
CTATGACGTAGTTGATctgtccatccaaccctggattgccttttggtaatctttggcgggagagcgaccgctcacgtgcg
taataaagaagaaattgaatggcatatgggctcctggtttcccctcaaactgataatagtccattgttctatcgagagttgtatatgcttctgt
cattagtacttttttttcccctctttctttgtgtagaaatcgaagacctccctgaaccttgttatcaatctatatgtattcggttggtccatcgt
ccgcgaccggttgtaattccagtagttcgtcgggtcgagatcgggcgagagcaattcttggtctcgccattTTACGTGCCGTT
ACACCGGTTACCGACATCAGGTTCCTTCAAATCGGACACGGGCGCCCCTCCCCGA
GGGGATGCCAATGGGGGGAGGTCCCAGGCCGAAGCCTGACTTTCTACTACCTCC
GGAGCTGTGCCCTTCTCTGCACGTCCCAGTTGAGCACTGGTGGGCTGACCTCGGG
GACAAGGTCGCCTTAACTTACCG
Luciferase-piRB-6
(SEQ ID NO: 12)
AGCAGCTTCTTGCCTCTGATTCCACGGTTTCTTCTTAAAGGGCCCCGACGACTGCT
GCGGGCCTTGATAAGGCGCGCTCCTGTTATTTGCCTCACGGAACGTCTTTTCCGC
GGCCATCATTGCGTCCATTGATCGGATCAAATCTTGCCTCATTGCATCCACGGCT
CGAGTATTCCTATCCGTATCCGCACGATTTAGATCAACTGCGTGTACCAAAGTCG
CTAGGGCGTTCTCATTGGCCTTCACCCGGGATTCTAAGGATGATTCCTGCCCCGT
ATAGTGATTTACGGCCAAAATAGCGCCCCTTCCTTTGCTGGTCGCGGCTACTGCT
AGCTTCGCATTttcgtgccagagtctttcgacagggacaaaaccattgccctgatcatgaacagctctgggtctaccggcctg
cctaagggcgtggccctgcctcatcgcaccgcctgtgtgcgcttctctcacgcccgcgaccctattttcggcaaccagatcatccccga
caccgctattctgagcgtggtgccattccaccacggcttcggcatgttcaccaccctgggctacctgattTCCGTCGAGTTA
ACTTTAGCCAAGCCCGCTAGTTTTCTCTTCGCTTGAACGTAATCCAACGGGTCCTC
ATTTTCTCCCTGCGTTCGCGCCGAGAATTTCGTGAGGGCATCCTCGTCGCTGTCAA
AGTATTGGATCAATTTCTTCTTTACTTCCTCAAAAGTCCTGCAGTTACCGAACGCT
ACCTCTTCATTGTCGTAGTACTGGATGGCACGTTTCGCTAAGTGATTTCTGAGTTG
GTCCCGCTTTTCTTGATCCGAACATTTCTTATAGAAATTTTCAAAATCTTTTAGAA
ATTCTCTAACGTCGTAGTCAGCTTCTCCTTTGAATAGTTTTCTAAACGGCGGTGCC
TTAATCGTCACCGTAGGT
Luciferase-No_bias-14
(SEQ ID NO: 13)
TTGCGTTCCTGCTCCCTTTGCCCTTTACCGCGCTCAATTATCTCTATTAGAACCGG
AGATATTCGGTTTACAAAAATTTTTTGGGGCCCAGCCCCCCTTAATCCTTTCCCTA
TGGACTTCCTATATGGCCCCAGAGGTAGCCCCCGGGGGTTAGGCAAATAATCCCA
AAAAATTCCCAAATTCTAACGGAAATGTGGCACTACCGCCCCTACGTCACTCTGG
CTATGACGTAGTTGATttcgtgccagagtctttcgacagggacaaaaccattgccctgatcatgaacagctctgggtcta
ccggcctgcctaagggcgtggccctgcctcatcgcaccgcctgtgtgcgcttctctcacgcccgcgaccctattttcggcaaccagatc
atccccgacaccgctattctgagcgtggtgccattccaccacggcttcggcatgttcaccaccctgggctacctgattTTACGTG
CCGTTACACCGGTTACCGACATCAGGTTCCTTCAAATCGGACACGGGCGCCCCTC
CCCGAGGGGATGCCAATGGGGGGAGGTCCCAGGCCGAAGCCTGACTTTCTACTA
CCTCCGGAGCTGTGCCCTTCTCTGCACGTCCCAGTTGAGCACTGGTGGGCTGACC
TCGGGGACAAGGTCGCCTTAACTTACCG
AQP1 sequence used in this study to synthesize dsRNA (from accession #KF377800.1)
(SEQ ID NO: 2)
Tcgcacaatgccttggagccatctgtggagcaatcattctgaatgaaatcacgccaaaaacaggttacacggctgctggtaatctggg
agtaacgacactgtctacaggagtttccgacctgcagggtgtggcgatagaagcactaatcacatttgtgctgcttttagttgtccagtcc
gtctgcgatgggaagcggaccgacatcaaaggatctatcggcgttgcgataggattcgcaattgct
AGLU1 sequence used in this study to synthesize dsRNA (from accession #KF377803.1)
(SEQ ID NO: 3)
Ctgtccatccaaccctggattgccttttggtaatctttggcgggagagcgaccgctcacgtgcgtaataaagaagaaattgaatggcat
atgggctcctggtttcccctcaaactgataatagtccattgttctatcgagagttgtatatgcttctgtcattagtacttttgttttcccctcttt
ctttgtgtagaaatcgaagacctccctgaaccttgttatcaatctatatgtattcggttggtccatcgtccgcgaccggttgtaattccagtagtt
cgtcgggtcgagatcgggcgagagcaattcttggtctcgccatt
Luciferase sequence was cloned from psiCHECKTM-2 plasmid (Promega, catalog #C8021)
(SEQ ID NO: 69)
Ttcgtgccagagtctttcgacagggacaaaaccattgccctgatcatgaacagctctgggtctaccggcctgcctaagggcgtggccc
tgcctcatcgcaccgcctgtgtgcgcttctctcacgcccgcgaccctattttcggcaaccagatcatccccgacaccgctattctgagcg
tggtgccattccaccacggcttcggcatgttcaccaccctgggctacctgatt
Primer sequences used in this study
(Underlined regions are overlap to the genes of interest)
piB6_A F
(SEQ ID NO: 48)
AGCAGCTTCTTGCCTCTGATTCCAC
piB6_BR
(SEQ ID NO: 49)
AATGCGAAGCTAGCAGTAGCCGC
piB6_CF
(SEQ ID NO: 50)
TCCGTCGAGTTAACTTTAGCCAAGCC
piB6_D R
(SEQ ID NO: 51)
ACCTACGGTGACGATTAAGGCACC
Eq14_A F
(SEQ ID NO: 52)
TTGCGTTCCTGCTCCCTTTGCC
Eq14_B R
(SEQ ID NO: 53)
ATCAACTACGTCATAGCCAGAGTGACG
Eq14_CF
(SEQ ID NO: 54)
TTACGTGCCGTTACACCGGTTACC
Eq14_D R
(SEQ ID NO: 55)
CGGTAAGTTAAGGCGACCTTGTCC
AQP1-piB6 F
(SEQ ID NO: 56)
GCTGGTCGCGGCTACTGCTAGCTTCGCATTTCGCACAATGCCTTGGAGCCATC
AQP1-piB6 R
(SEQ ID NO: 57)
AGCGGGCTTGGCTAAAGTTAACTCGACGGAAGCAATTGCGAATCCTATCGCAAC
G
AQP1-Eq14 F
(SEQ ID NO: 58)
CTACGTCACTCTGGCTATGACGTAGTTGATTCGCACAATGCCTTGGAGCCATC
AQP1-Eq14 R
(SEQ ID NO: 59)
GATGTCGGTAACCGGTGTAACGGCACGTAAAGCAATTGCGAATCCTATCGCAAC
G
AGLU1-piB6 F
(SEQ ID NO: 60)
GCTGGTCGCGGCTACTGCTAGCTTCGCATTCTGTCCATCCAACCCTGGATTGCC
AGLU1-piB6 R
(SEQ ID NO: 61)
AGCGGGCTTGGCTAAAGTTAACTCGACGGAAATGGCGAGACCAAGAATTGCTCT
CG
AGLU1-Eq14 F
(SEQ ID NO: 62)
CTACGTCACTCTGGCTATGACGTAGTTGATCTGTCCATCCAACCCTGGATTGCC
AGLU1-Eq14 R
(SEQ ID NO: 63)
GATGTCGGTAACCGGTGTAACGGCACGTAAAATGGCGAGACCAAGAATTGCTCT
CG
AGLU1_dsRNA F
(SEQ ID NO: 64)
CTGTCCATCCAACCCTGGATTGCC
AGLU1 dsRNA R
(SEQ ID NO: 65)
AATGGCGAGACCAAGAATTGCTCTCG
Aqp1_dsRNA F
(SEQ ID NO: 66)
TCGCACAATGCCTTGGAGCCATC
Aqp1_dsRNA R
(SEQ ID NO: 67)
AGCAATTGCGAATCCTATCGCAACG

238- and 199-nt-long region from No_bias-14 locus (Scaffold40734: 1537-1774, 1811-2009) were fused to the left and right sites of the GOI, respectively. From the piRB-6 locus, the left and right flanking sequences were 342 and 366 nt, respectively (Scaffold185: 15168-15509 and 15616-15981). All Six fusion constructs were cloned into pGEMT-easy plasmid for double-stranded and ssRNA synthesis. 231-nt luciferase gene sequence from psiCHECK-2 (Cat. no. C8021; Promega) vector was cloned into the pGEMT-easy vector. ssRNA and dsRNA from the luciferase sequence was used as control RNA.

Each of the piRNA trigger constructs consisted of three parts, which were PCR-amplified from whitefly cDNA using Phire Plant Direct PCR Master Mix (Cat. no. F160S) following the manufacturer's instruction. During these PCRs, 30-nt sequence from the left and right flanking regions were added to the GOI (AQP1, AGLU1, and Luciferase) sequences by adding the sequences in the forward and reverse primers of the GOI. Gel-extracted PCR products (GeneJET Gel Extraction Kit, Cat. no. K0691) were then ligated using two separate SOEing PCRs. First, the left flanking sequence was attached to the GOI and gel-extracted. In the second step, the fusion product from the first step was ligated to the right flanking sequence. These sequences are provided above.

PCR products with T7 promoter sites on both strands were used for dsRNA synthesis, whereas for ssRNA, PCR was carried by allowing the T7 promoter site in one strand. PCR products were directly used to synthesize the synthetic RNAs using MEGAscript T7 Transcription Kit (Cat. no. AM1334; Thermo Fisher Scientific) following the manufacturer's protocol.

Oral Delivery of the Synthetic RNAs to Whitefly, RNA Extraction, and qRT-PCR

Using a hand-held aspirator, 100 adult whiteflies were collected for each biological replicate from the colony and transferred to a plastic feeding chamber. 200 μl of 30 ng/μl RNA in 20% sucrose solution was sandwiched between two sterile Parafilm M layers, and feeding access to the solution was given to the insects for 6 d. On day 6, the insects were collected for RNA extraction. Total RNA was extracted following the standard TRIzol RNA extraction method. The extracted RNAs were DNase I-treated (DNA-Free kit, Lot 00522653; Invitrogen) and 2 μg RNA was used for cDNA synthesis using High Capacity cDNA Reverse Transcription Kit (Lot 00692533; Applied Biosystems). The TaqMan qPCR master mix (Universal PCR Master Mix, Lot #1908161; Applied Biosystems) was used for quantitative gene expression analysis using standard protocol. Whitefly 18S ribosomal RNA gene was used for normalizing the expression of the target genes. All qRT-PCR primer sequences are shown above. Each treatment and control groups of the synthetic RNA feeding were carried out using at least three independent biological replicates. The ΔΔCt method was used for gene knockdown analysis. t test and one-way ANOVA were used for statistical analysis in CFX Maestro software v1.1.

mRNA Library Preparation, Sequencing, and Gene Expression

Total RNAs were extracted using conventional the TRIzol RNA extraction method from different manually dissected tissues of whiteflies (gut, salivary gland, and whole body) (Cicero & Brown, 2011). RNA integrity was confirmed using an Agilent 2100 Bioanalyzer (Agilent Technologies Inc.). Sequencing libraries were constructed using Illumina's TruSeq RNA Sample Preparation Kit v2, Cat. no. RS-122-2002 (Set B). Using magnetic oligo (dT) beads, only poly(A) tail containing RNAs were separated from total RNA. Next, the mRNAs were fragmented by zinc treatment, and the first-strand cDNA was synthesized from the fragmented RNAs using SuperScript II reverse transcriptase and random primers from Invitrogen. Then second-strand cDNA was synthesized, and Illumina multiple indexing adapters were ligated to the fragments. The remaining library construction steps were carried out following the manufacturer's protocol. Quality filter and processing of the sequenced reads were performed using Illumina CASAVA v1.7.0, FastQC, and Trimmomatic. For each of the RNAi factors analyzed (FIG. 1B), the reads were mapped with Bowtie2 to transcript sequences from the whitefly genome database www.whiteflygenomics.org (Langdon, 2015; Chen et al, 2016). bedtools was used to count read alignments to each transcript (Quinlan & Hall, 2010).

### Small RNA Library Preparation and Sequencing

Total RNA was extracted from adult whiteflies using the standard TRIzol RNA extraction protocol. After the DNase treatment, small RNA-seq libraries were constructed using NEXTflex Small RNA-Seq Kit v3 (NOVA-5132-06). First, A 39 4N adenylated adapter was ligated to the 39 end and 59 standard Illumina adapter was ligated to the 59 end of the RNAs. Reverse transcription was carried out on the adapter ligated RNAs. Synthesized cDNAs were PCR-amplified, and each sample was barcoded with 17 Illumina-compatible in-line barcode. PCR products were cleaned up by NEXTflex cleanup beads, and size selection of the DNAs was performed on a Sage Scientific Blue Pippin. Sequencing was carried out on a 1×75 flow cell on the NextSeq 500 platform (Illumina) at the Arizona State University's genomics core and on a 2×150 flow cell NovaSeq platform at the genomics core, University of Colorado, Denver.

### Bioinformatics Pipelines Used for Small RNA Analysis

Small RNA reads were quality checked using FastQC, and the adapter sequences were cleaved and trimmed using FASTX toolkit. Next, 15-35-nt size reads were mapped to whitefly genome (MEAM1 genome v1.2) using Bowtie with default parameters (Chen et al, 2016). The genome-mapped reads were isolated for the downstream analysis. mirDeep2 was used to annotate the miRNAs (Friedlander et al, 2008). Initial calls by the algorithm were manually inspected for recognized features of miRNAs (Berezikov et al, 2010). Annotations that showed evidence of mature and star strands in the appropriate Dicer cleavage register as well as significant expression were placed in the confident category. Deviation from these characteristics resulted in placement of annotation in the candidate category. For non-miRNA annotations, small RNA reads, either taking all reads, 19-23-nt sized reads, and 25-30 nt reads were aligned using Bowtie multi-mapping (-a -m 100) options. Bowtie was also used to identify the targets by allowing three mismatches. Size distributions were calculated with basic unix commands: awk, sort, uniq, etc. Using Bowtie alignments ping-pong overlap, piRNA phasing, and Dicer siRNA overhangs signatures were calculated as previously reported (Antoniewski, 2014; Han et al, 2015). SAMtools and bedtools were used to count read alignments and identify high-expressing regions and bias toward short and long read loci, as well as determine potential targets (Quinlan & Hall, 2010). The R packages Scatterplot3d, sushi, heatmap2, pheatmap, and ggplot2 were used to draw the read density graphs (Kolde, 2012; Phanstiel et al, 2014; Wames et al, 2016; Wickham, 2016; Ligges et al, 2018). The seqlogo program was used to visualize nucleotide biases (Crooks et al, 2004). Read subsetting based on sequence content was carried out using standard Linux tools (grep, awk, etc.)

### Results:

### Whitefly RNAi Pathways

Because of the divergent nature of siRNA and piRNA biology, species-specific design is necessary to fully exploit these pathways for effective gene silencing. To characterize the RNAi pathways of whitefly, the collection of Ago/Piwi proteins encoded in the whitefly “B biotype” (also known as MEAM1) genome (MEAM1v1.2) were identified using existing annotations and BLAST to curate sequences (FIG. 1A) (Chen et al, 2016). These sequences were then compared with Ago/Piwi proteins from Drosophila melanogaster, Tribolium castaneum (red flour beetle), and subset from Caenorhabditis elegans. Seven members of this family encoded in the whitefly genome were found. Three of the genes belong to the Ago family with one clearly related to miRNA-loading factors. The two other Agos group with the siRNA-associated Agos appear to be a clade specific duplication as they are not homologs of the two si-Agos seen in T. castaneum. This is consistent with the diverging nature of siRNA biology and opens up the possibility that si-Ago function in whitefly might be distinct from fruit flies and beetles. The other four members belong to the Piwi clade. One of the whitefly Piwi proteins is a homolog of DmeAgo3, whereas the other three groups with DmePIWI/DmeAub. This indicates that the ping-pong biogenesis is likely present in these animals. The phasing piRNA pathway is also presumably operative with apparent homologs of Zuc (Bta02312) and the RNA helicase Armitage (Bta07189) (Ishizu et al, 2019).

piRNAs are found in somatic tissues of many insect orders, including hemipterans (Huang et al, 2017; Lewis et al, 2018). To verify if this is also pertinent for whitefly, RNAi factor expression was evaluated in the whitefly guts, salivary glands, and whole body (FIG. 1B) (Cicero & Brown, 2011). PolyA sequencing libraries from extirpated whitefly guts, salivary glands, and whole body were mapped to the RNAi factor sequences from MEAM1v1.2, per above (Chen et al, 2016). The alignments were then used to calculate Reads Per Kilobase of transcript, per Million mapped reads (RPKM) values for each transcript. Expression of BtaAgo1 and BtaAgo2 was found in all tissues, as well as two Piwi's (BtaPiwi3 and BtaAgo3), along with BtaZuc and BtaArmi (FIG. 1B). This contrasts with BtaPiwi2, which is enriched in whole body presumably because of the inclusion of RNAs originating from gonad tissues. This suggests that similar to other hemipterans, whiteflies have somatic piRNAs with both ping-pong and phasing piRNA biogenesis modes being present. Significantly, somatic piRNAs are likely present in whitefly gut, the tissue that would be the primary target of ingested RNAi trigger molecules.

To further investigate whitefly RNAi pathways, endogenous small RNA populations from whole body mixed adults (male and female) were examined using small RNA sequencing libraries mapped against MEAM1v1.2. From this alignment, miRNAs were first annotated using miRDeep2 (Friedlander et al, 2012). Subtracting miRNA-derived reads from datasets would allow focus on non-miRNA small RNA loci such as endo-siRNAs and piRNAs, which unlike miRNAs might have whitefly specific biology. 202 miRNAs were identified with high confidence with 89 being conserved in Drosophila (FIG. 1C). 124 additional miRNAs were also identified, which were classified as lower confidence miRNA candidates because of suboptimal features such as low expression or imprecise precursor cleavage patterns. The miRNA repertoire of the whitefly genome is similar in size to other insects (Kozomara et al, 2019). These results expand on prior miRNA annotations in whitefly because of the increased depth of datasets featured in this study.

Next, size distribution of reads was examined and a bimodal read size distribution was found with peaks at 22 nt representing Dicer products (siRNAs and miRNAs) and 29-30 nt (piRNAs) (FIG. 1D). Among the Dicer products, roughly 56% derive from miRNAs. This shows piRNAs are more abundant relative to siRNAs in whitefly. To examine the modes of piRNA production, the abundance of read overlap pairs and the distance to 1U trailing reads was analyzed (FIG. 1D). During ping-pong biogenesis, piRNA pairs are cleaved at the 10th base of the guiding RNA. Thus, when this mode is active, piRNAs are found to overlap by 10 bases, which is clear in the dataset. Phasing piRNAs are biased to occur end-to-end and can be recognized by close proximity of trailing reads. Phasing is also evident in the alignment. The abundance of piRNAs is further reflected by the high-proportion 1U reads in the size distribution. Simultaneously, a significant proportion of the reads also exhibit an “A” at the 10th base which would be found on ping-pong pair reads because of pairing with 1U.

### Non-miRNA, Small RNA-Producing Loci in Whitefly

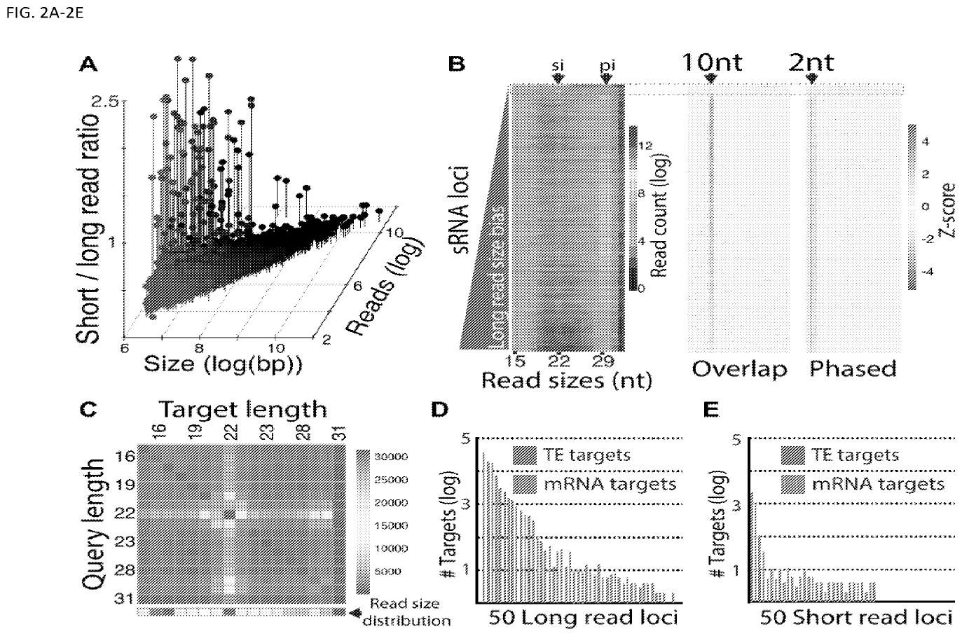

Using reads subtracted of miRNAs, non-miRNA, small RNA-producing loci were annotated. 3,873 regions were identified with a read depth greater than 40 and 500+ bp length (FIG. 2A). The ratio of the number of small (19-23-nt) to long reads (25-30-nt) was then calculated to distinguish whether the locus produced smaller siRNAs (19-23-nt) or longer piRNAs (25-30-nt). This showed the majority of loci appear to be piRNA generating. Only 50 loci had a ratio of small to long that was greater than “one.” Interestingly, the piRNA loci spanned regions ranging in size from 500 nt to 50 kb, indicating diverse transcripts generate this small RNA class (Yamanaka et al, 2014). Apparent siRNA-producing loci tended to be shorter regions, of which the longest was about 4 kb in size. The distribution of read sizes at each locus was next examined (FIG. 2B). Accumulation was most clear in the piRNA range, which was substantially less for siRNAs. However, a minor signature of siRNA-sized reads could be seen at many loci. To confirm that most loci were sites of piRNA production, read overlaps and trailing 1U read distance were examined, which shows evidence of ping-pong due to 10-nt overlap bias and phasing with juxtaposed trailing 1U reads, respectively. The exception was ˜100 loci that showed a greater accumulation of siRNAs.

To verify if these loci are sources of Dicer-produced siRNAs, the 2-nt overhang signature of RNase III processing (FIG. 2C) was investigated. Overlapping read pairs between 15 and 31 nt with this signature were quantified. Pairs were identified where one strand (query read) of a potential duplex overlapped by less than two of its entire length, which would occur with a 2-nt overhang (Antoniewski, 2014). All potential combination of query and complementary target reads were quantified, revealing that 22-nt reads show the greatest evidence of Dicer processing and that this is likely the size of Dicer-2 products. The abundance of apparent Dicer overlapping reads differed from the distribution of the reads in different size ranges, validating this method of characterizing biogenesis. Interestingly, some signal could also be seen in the 29-30-nt sizes that likewise were not reflected in the all read size distribution. This suggests a potential interaction between siRNAs and piRNAs, unlike what is reported in Drosophila, and which is consistent with the frequent co-occurrence of siRNA-sized and piRNA-sized reads across all annotated loci (FIG. 2B).

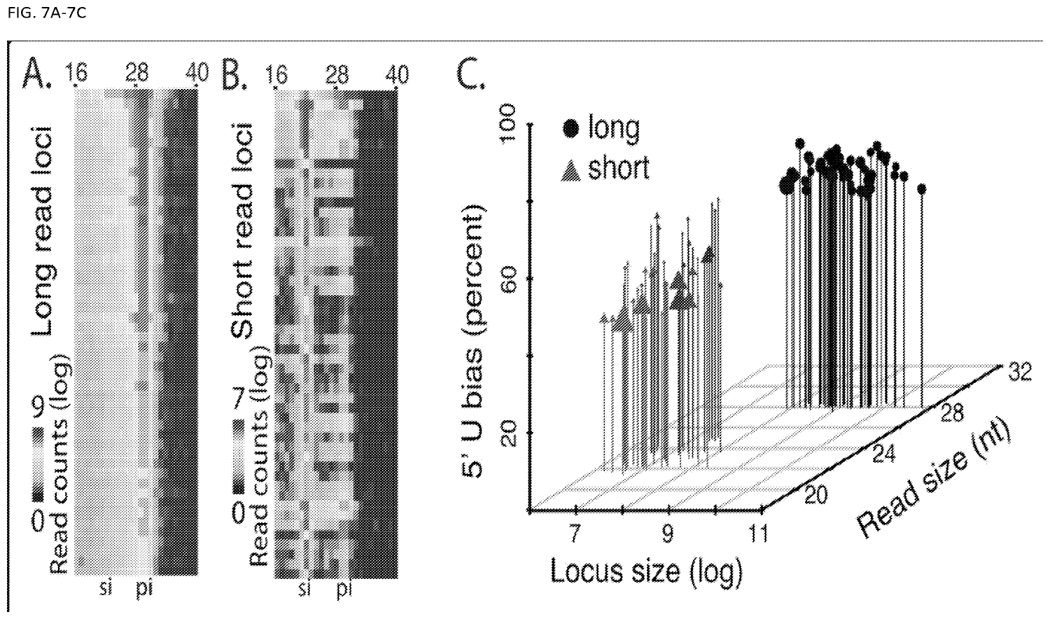

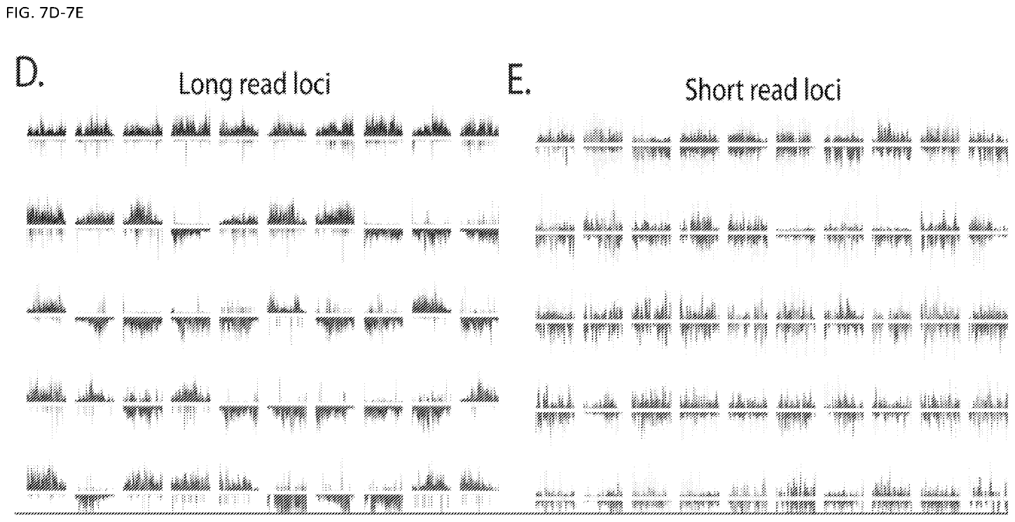

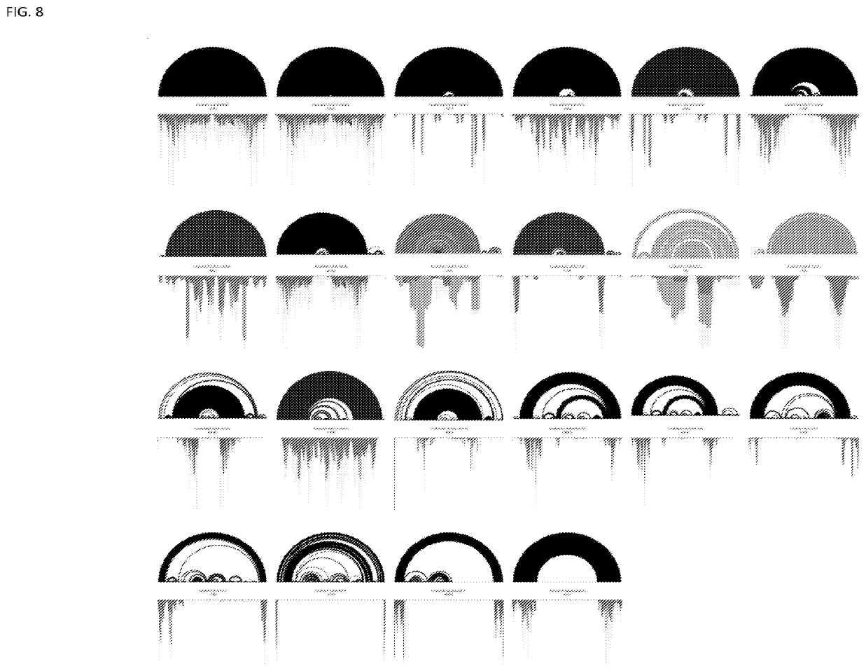

Next, assessment was focused on the filter loci by expression to focus on the top 50 long read biased or short read biased loci (FIG. 7). The size distribution of reads for each locus was determined, which showed 28-30-nt reads at long read loci, consistent with production of piRNAs (FIG. 7). This contrasts with the short read loci, which show signal at 22 and 29 and 30-nts, consistent with co-occurrence of piRNAs and siRNAs seen across all loci (FIG. 2B). The two groups of loci were characterized by length, expression, and 1U bias (FIG. 7). This showed that long read loci are larger, have a greater bias toward 1U, and greater expression, which are characteristics of piRNA clusters. Examining strand mapping showed that high expressing long read loci exhibit bias toward small RNA production from one strand indicating likely single-stranded precursor transcripts converted into phasing piRNAs (FIG. 7) (Gainetdinov et al, 2018). The short read loci are predominantly dual-stranded, which is suggestive of a dsRNA precursor serving as a substrate for Dicer (Claycomb, 2014). 22 hpRNA loci were also identified, indicating that this variety of locus is present as a minority of the overall collection of whitefly siRNA-generating loci (FIG. 8).

To predict the function of these 100 loci, reads aligning to these loci were mapped back to the whitefly genome permitting up to three mismatches. This alignment was then intersected to MEAM1v1.2 annotations (FIGS. 2D and E). The number of intersections was determined for each locus keeping mRNAs and TEs separate. Both long and short reads target mRNAs and TEs indicating possible roles for piRNAs and siRNAs not only in genome surveillance but also in gene regulatory networks. This is consistent with a proposed role for piRNAs in regulation of protein coding gene expression (Shamimuzzaman et al, 2019). Taken together, this suggests that whitefly siRNAs and piRNAs are gene regulatory factors alongside miRNAs. These observations further reinforce the potential for exploiting these pathways for genetic technology that silences genic transcripts.

Whitefly Endo-siRNA Loci are Also Sources of piRNAs

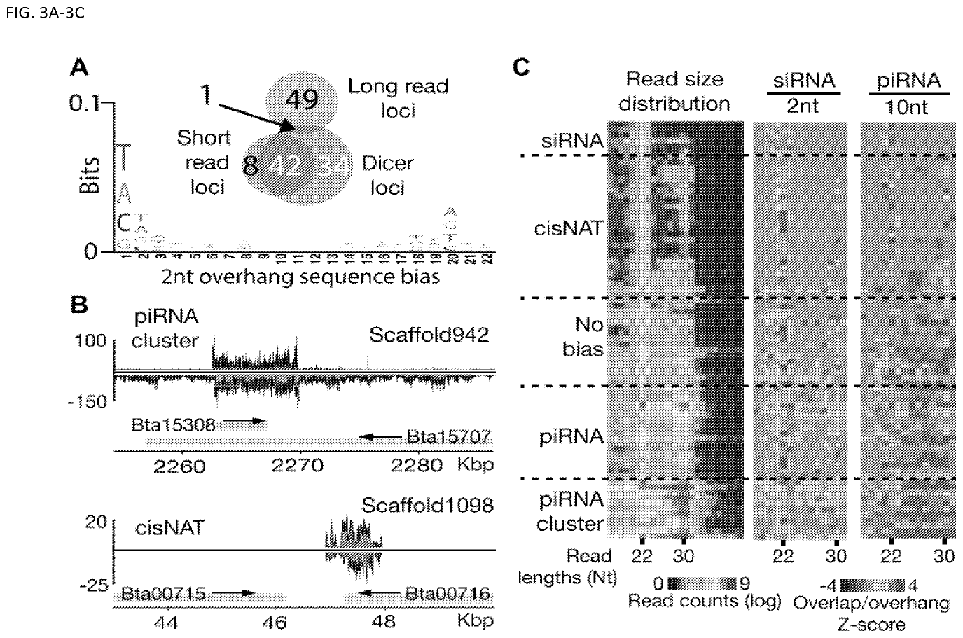

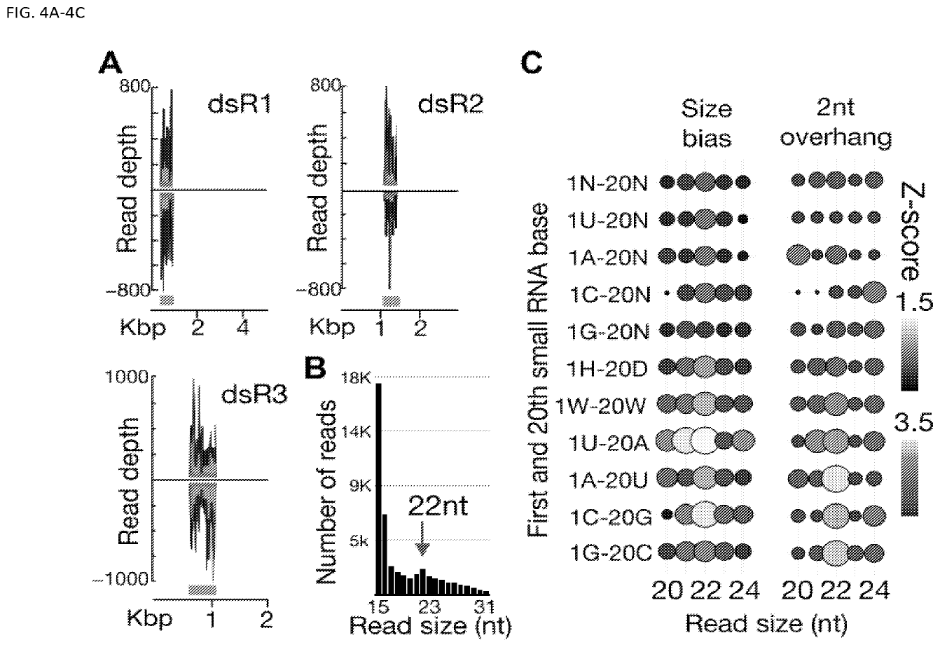

To better understand small RNAs simulated by fed dsRNA, the computational approach described above that finds the 2-nt overhang signature of RNase III cleavage in 20-23 nt reads was used. Based on this, 76 loci exhibiting apparent Dicer processing were annotated (FIG. 3A). When intersecting these Dicer loci with the high expressing long and short read loci (FIG. 7), 42 short read loci and only one long read locus have the Dicer processing signature (FIG. 3A). Seqlogo analysis was also performed on the 22-nt Dicer reads showing a bias for 1U and a matched 20th base A (20A). Among the other bases, 1G residues were disfavored along with the paired 20C.

Next, individual loci were inspected to understand their function and biogenesis. The Dicer locus that overlapped with the one long read locus is an interesting genomic site (FIG. 3B). This region is a large phasing piRNA precursor with an annotated, interior antisense transcript. The Dicer signature reads coincide with this antisense transcript that seems to form a dsRNA with the piRNA precursor. Other Dicer loci also arise from overlapping antisense transcripts. Indeed, many cis-NAT siRNAs are observed in the Dicer annotations, with one such example shown from Scaffold1098 (FIG. 3B).

Through curation of the annotations, loci were placed in five categories: siRNA, cis-NAT, No bias, piRNA, and piRNA cluster (FIG. 3C). These groupings were determined by evaluating dominant small RNA size and the dominant processing signatures of read pairs-2-nt overhangs for Dicer or 10-nt overlaps for ping-pong piRNA. The siRNA group is located in intergenic regions and has a strong bias toward short reads that appear to be dicer processed. The cis-NATs were sites of siRNA production between opposing mRNAs as showing in FIG. 3B. In addition to these predominantly siRNA-producing regions that are similar to ones observed in Drosophila (Czech et al, 2008; Ghildiyal et al, 2008), many loci produce both siRNAs and piRNAs. These dual-identity loci could be grouped into one of three categories. One where there was equal production of siRNAs and piRNAs (No bias), a second for which some siRNAs were present, but piRNAs are dominant (piRNA), and the third group that harbors large piRNA clusters with only a minor production of siRNAs, the latter being similar to the locus shown in FIG. 3B. These observations show that despite an Ago repertoire similar to Drosophila, small RNA biogenesis in whitefly is distinct. This provides an opportunity to exploit these divergent activities for gene silencing and pest management.

Metabolism of Exogenous dsRNA by Whitefly

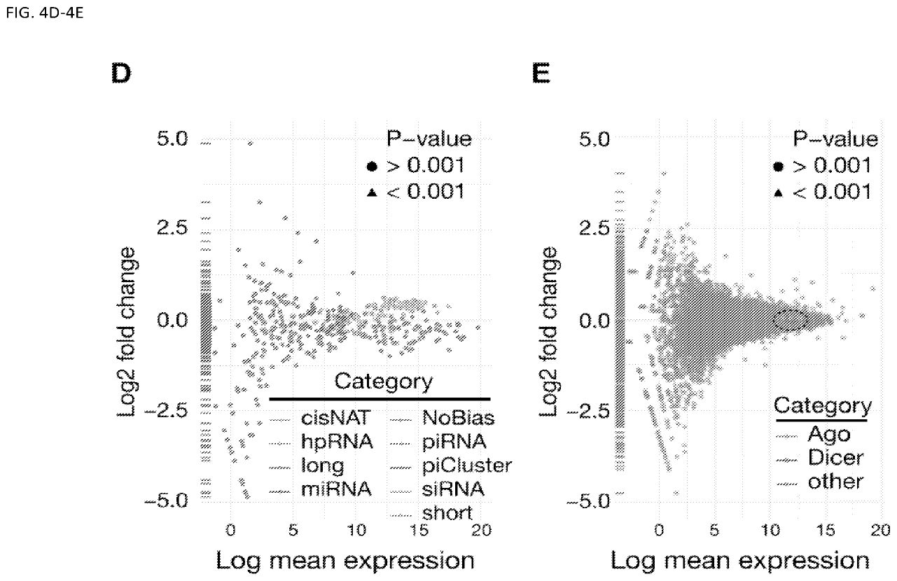

The evaluation of processing dsRNA transcripts was extended to those introduced exogenously via feeding. Here, three off-target, synthetic dsRNAs dissolved in a sucrose solution fed through an artificial system were tested. The RNAs cloned from genes of the potato psyllid Bactericera cockerelli (Sulc.) were fed to adult whiteflies from which small RNA and messenger RNA sequencing libraries were generated. Significant accumulation of reads arose exclusively from dsRNAs and not from other sections of the psyllid gene from which they were cloned (FIG. 4A). However, only a fraction of the reads show a signature of Dicer processing based on 2-nt overhangs. This suggests that most of the synthetic RNA was likely degraded with only a minority entering the siRNA pathway, which is reflected in the distribution of reads produced from the dsRNA sequences (FIG. 4B). Only a modest peak was seen at 22 nt with many more at the 15 nt size. The low efficiency is likely in part caused by dsRNA-specific nucleases (dsRNases), which are common in hemipteran insects. Several dsRNAses from gut and other tissues have been identified in whiteflies (Luo et al, 2017; Singh et al, 2017). Furthermore, the alkaline pH of stemorrhynchan midgut is also compromised by RNA integrity (Cristofoletti et al, 2003; Molki et al, 2019).

Using these datasets, similarities between small RNAs derived from fed dsRNA and endogenously expressed siRNAs were examined. Specifically, reads were subsetted based on the sequence content to find population where signatures of dicer processing were most evident (FIG. 4C). This was guided by the seqlogo results of endogenous siRNAs that showed preference for 1U and depletion of 1G (FIG. 3A). In unfiltered reads, only a slight enrichment of 22-nt RNAs was seen with no evidence of 2-nt overhangs. Next, the reads were extracted based on their 59 residue, which showed similar size distribution to the unfiltered library with the exception of 1G reads where there was no bias toward 22-nt reads. However, for each subset, no 29 overhang specific to 22-nt reads was observed. The analysis was then extended to include not only the first base of the read but also the 20th base. When considering 1U/A/C(H) and 20A/U/G(D), greater abundance of 22-nt reads was seen but still no substantial 2-nt overhang Dicer signature. When 1U/A(W) and 20A/U(W) were examined, an even greater enrichment of 22-nt reads as well as 2-nt overhang enrichment for this size was observed. For individual nucleotide pairs (1U-20A, 1A-20U, 1C-20G, and 1G-20C), read size and overhang enrichment increased, particularly for 1U-20A in size distribution and 1A-20U for 2-nt overhang. This analysis provides a framework for computationally isolating siRNA processing signatures from degradation products, which is useful when considering exogenous dsRNA processing because of cloning of digestive contaminants.

To understand the physiological consequences of ingesting dsRNA, the effect on expression of the small RNA loci annotated in this study and protein-coding genes was investigated. After feeding dsRNA, small RNA sequencing showed no significant change in endogenous small RNA expression compared with control (FIG. 4D). For protein coding genes, about 500 transcripts that were differentially expressed based on a P-value≤0.001 (FIG. 4E) were observed. Only 20 of these genes exhibited a log(fold2) value greater than 2 or less than −2. All genes in this group have very low expression with 14 having unknown function. The genes having a known identity appear to be involved in basic metabolism or development. The one exception is an RNase H-containing gene (Bta15726) that could be involved in an antiviral response. However, it is downregulated, which is inconsistent with being deployed to combat perceived viral infection. Thus, it would seem that whitefly does not mount an antiviral-type response to dsRNA. No change in expression of RNAi factors such as the Ago and Dicer proteins was observed, suggesting that whitefly can metabolize exogenous RNAi triggers without affecting its core RNAi processes. Taken together, it appears that when ingested, the bulk of dsRNA is degraded with a small amount contributing to the siRNA pool and that exposure to dsRNA has a minimal impact on off-target gene expression in whitefly.

Exploiting Somatic piRNAs in Addition to siRNAs for Gene Silencing

In this study, a significant population of piRNAs were found, which are more abundant than the endogenous siRNAs—the species exploited by existing RNAi approaches. The piRNAs also appear to be expressed in soma and show potential widespread control of mRNAs and not just a role in genome surveillance. This suggests that the piRNA pathway might be exploited to silence endogenous gene expression in whiteflies as an alternative method to the classic dsRNA-based siRNA strategy.

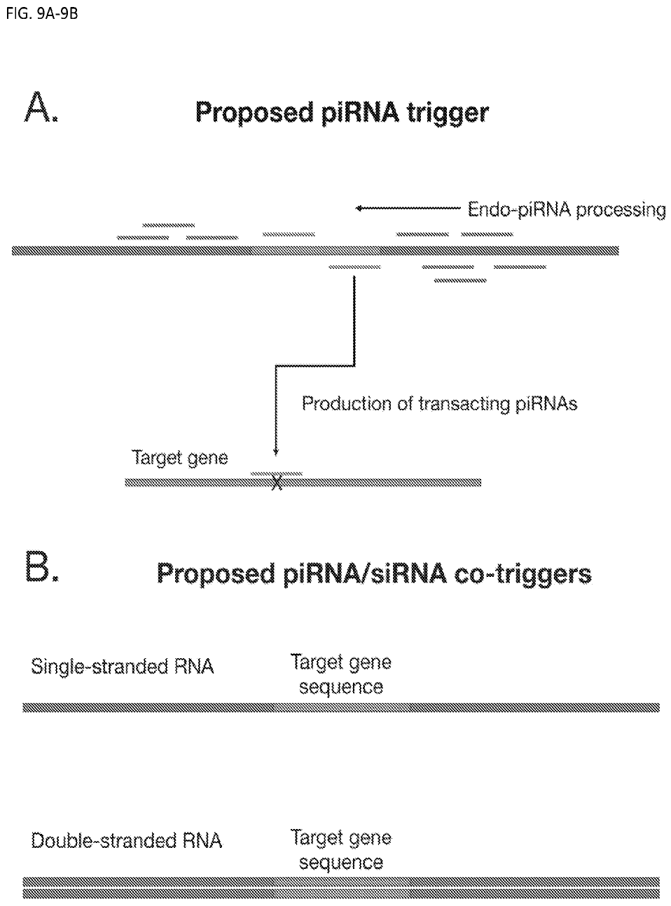

To trigger ectopic production, recombinant nucleic acids were engineered that take advantage of the major principle of piRNA biogenesis-recruitment of Piwi-cleaved fragments into the pathway (FIG. 9). Sequences from two loci annotated in this study were fused, a piRNA bias locus (piRB-6) and siRNA-piRNA no bias locus (No bias-14) to target gene sequences. Both loci were among those that showed evidence of Dicer processing as well as piRNA production (FIG. 3). Two different genes to target with these constructs were chosen: aquaporin1 (AQP1) and alpha glucosidase1 (AGLU1). To explore design principles, the positive strand of the locus was fused to AQP1 and the negative strand to AGLU1.

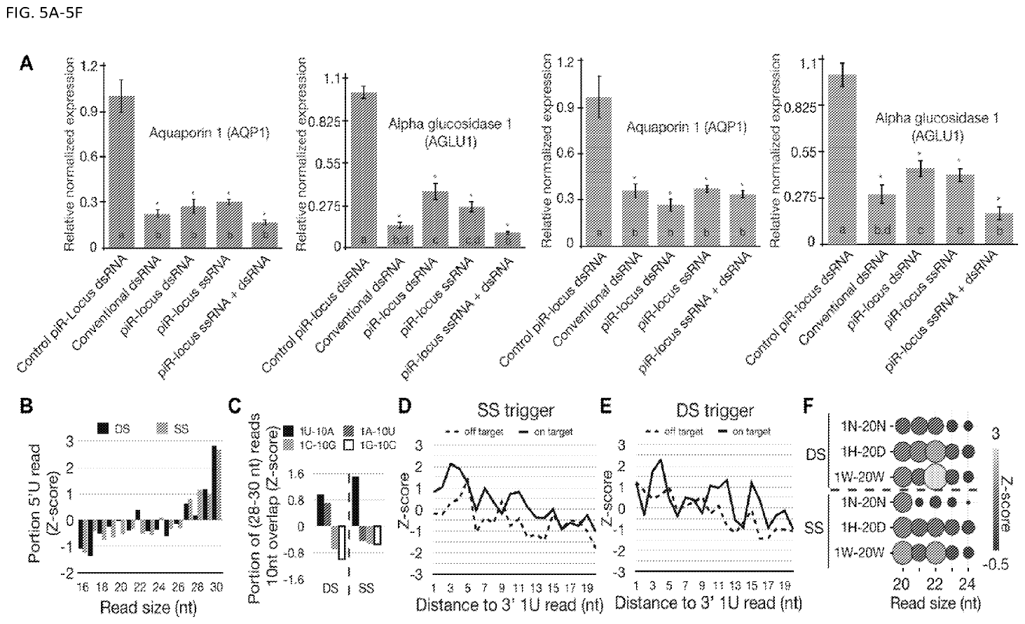

Using these constructs, both synthetic dsRNAs and ssRNAs (single-stranded RNA) were generated and fed to whiteflies in the artificial system described above. The concentration of RNAs (30 ng/μl) was used. Luciferase sequences fused to piRB-6/No_bias-14 were used as off-target controls. After feeding access for 6 d, expression of target genes was assessed by qRT-PCR (FIG. 5A). dsRNAs elicited gene knockdown of 68-80%. Satisfyingly, the piRNA triggers showed a similar degree of gene silencing with reduction in target expression of 60-80%. This result was observed for both ssRNA and dsRNA triggers with both piRNA sequences (piRB-6, No_bias-14) and targets (AQP1, AGLU1). For AQP1, piRNA triggers were equal to dsRNA (conventional dsRNA), whereas AGLU1 was not as well down-regulated by the piRNA triggers relative to dsRNA, suggesting inclusion of positive strand sequence might lead to superior knockdown. However, by combining ssRNA and dsRNA piRNA triggers for either sequence, gene silencing became comparable to conventional dsRNA for AGLU1. These results provide robust evidence that piRNA triggers, even those that comprised ssRNA, are capable of gene silencing in organisms that share RNAi biology with whiteflies.

Small RNAs were then sequenced to characterize the processing of the piRNA triggers. Small RNAs were sequenced from animals fed piRB-6 dsRNAs and ssRNAs targeted to both AQP1 and AGLU1 (FIGS. 5B-F and 6). Reads mapping to these triggers showed significant heterogeneity in read size with no accumulation of a specific size, indicating the bulk of fed RNAs were degraded. To identify potential small RNAs among the detritus, the relative abundance of 1U reads was determined, a characteristic of piRNAs as well as siRNAs (FIG. 5B). From this, significantly more 26-30-nt piRNA-sized reads were found. In the double-stranded treatment, a small peak possibly corresponding to 22-nt siRNAs could be observed, but not for the single-stranded piRNA triggers.

Next, focus was placed on the identity of small RNAs produced against the target gene. piRNA biogenesis could be observed for both triggers but more so for the single-stranded versions (FIG. 5C). Ping-pong processing was observable when comparing the number of overlaps for different nucleotide combinations that can form pairs: 1U/10A, 1A/10U, 1C/10G, and 1G/10C. Read pairs were determined for all ranges of reads, and for those in piRNA sizes (28-30 nt). The greatest enrichment for 28-30-nt reads was seen for those with the signature of ping-pong piRNAs: 1U/10A. Phasing was also assessed for each strand of the piRNA triggers (FIGS. 5D and E). This biogenesis mechanism was evident for the transcribed strand of single-stranded triggers, which is complementary to the target genes (AQP1 and AGLU1). For both strands of the double-stranded trigger and the potential target-derived reads in the single-stranded fed condition, less phasing was evident, although a noticeable trend toward close proximity of 1U reads was seen.

Next, it was investigated whether siRNAs were processed from the triggers by examining 2-nt overhangs in read populations as in FIG. 4C. When reads with 1U/A/C-20U/A/G or 1U/A-20A/U were examined, the double-stranded trigger showed a greater number of 22-nt Dicer signature reads (FIG. 5F). Together, these results show that regardless of whether the trigger is double-stranded or single-stranded, piRNAs are produced. However, there is less piRNA production from dsRNAs. Presumably, accessing the piRNA pathway requires an unwinding step for dsRNAs mediated by gut or cellular helicases, reducing the entry of double-stranded triggers. In comparison, the double-stranded triggers give rise to more production of siRNAs.

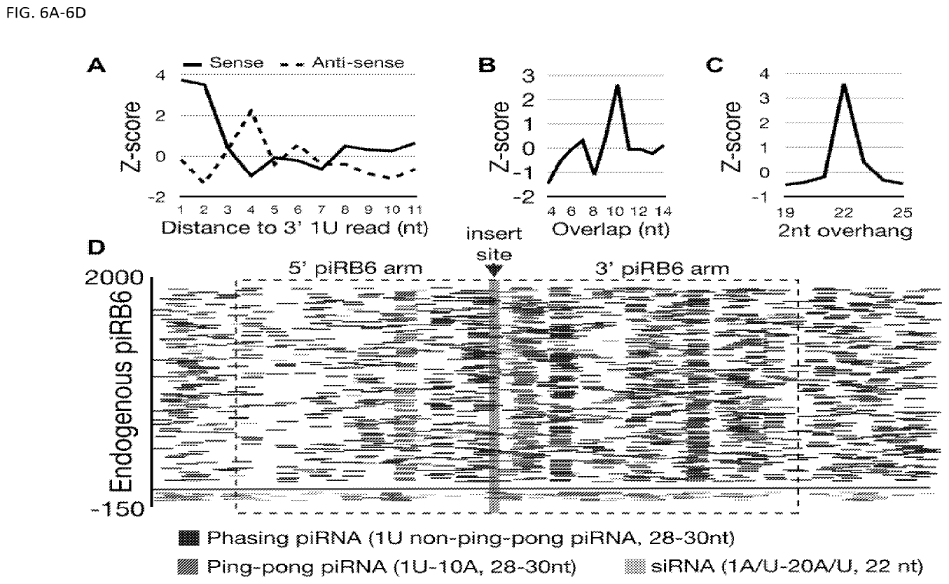

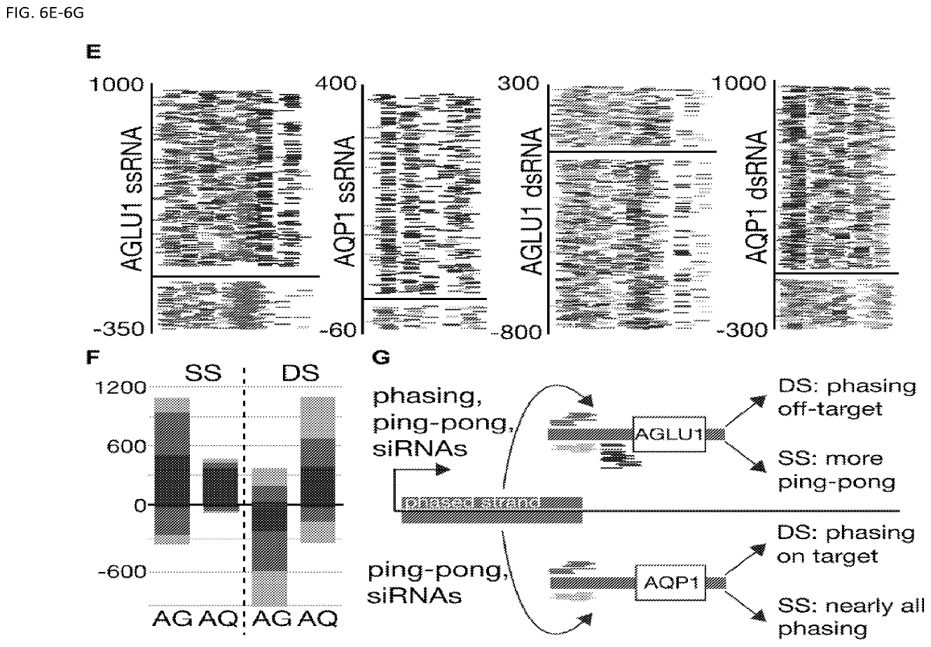

To understand the differences in target knockdown by the different piRNA trigger configurations, biogenesis of small RNAs from each was investigated. Before examining the exogenous triggers, small RNA production from the endogenous piRB-6 locus used to make the piRNA triggers was investigated more deeply (FIG. 6A-D). This region shows clear piRNA phasing, ping-pong, and siRNA biogenesis (FIG. 6A-C). Read alignments of each biogenesis mode were visualized at the locus (FIG. 6D). 28-30-nt 1U-10A reads overlapping by 10 nt represent ping-pong reads. Phasing piRNAs are reads 28-30-nt long with a 1U that did not show a 10-nt overlap. siRNAs are reads that start with 1U/1A and a 20A/20U also showing a 2-nt overhang. At this locus, the positive strand of the locus shows nearly 20-fold accumulation of small RNAs. This is clearly due to phasing piRNAs on the positive strand, and only modest accumulation of ping-pong piRNAs and siRNAs on the negative strand.

The asymmetry of read expression at the piRB-6 locus appears to cause the difference in gene silencing for the two configurations of piRNA triggers (FIG. 5A). This is apparent when the accumulations of small RNA types are examined for the gene targeted region of each trigger (FIG. 6E-G). As with the endogenous loci, 1U-10A ping-pong piRNAs, 1U non-ping-pong piRNAs (phasing), and siRNAs with 1U/A-20A/U were quantified. The AGLU1 trigger is composed of the antisense of piRB-6 and could be targeted by sense-phasing piRNAs, ping-pong piRNAs, and siRNAs. For the single-stranded version of the AGLU1 trigger, significant accumulation of ping-pong piRNAs and phasing on the strand synthesized and fed was observed. The ping-pong piRNAs complementary to the trigger are likely derived from the target gene, which is robustly silenced by this trigger. This contrasts with the double-stranded AGLU1 trigger, which shows that the off-target strand is much more robustly converted into small RNAs, particularly presumptive phased piRNAs. This explains the lower silencing efficiency for double-stranded AGLU1 trigger (FIG. 5A). The off-target strand of the AGLU1 dsRNA trigger duplex is the strand that is phased in the endogenous locus.

This same phenomenon is seen in AQP1 triggers which sport the sense strand of piRB-6 for the on-target strand. For single-stranded AQP1, nearly all the RNAs appear to be phasing piRNAs, and for the double-stranded version, most of the phasing piRNAs are on-target. Both of these trigger versions lead to robust gene silencing. These results indicate that a superior choice for piRNA trigger design is to select the phased strand of piRNA loci to fuse with gene-targeting sequences. It also shows that the small population of endogenous antisense ping-pong piRNAs or possibly even the siRNAs has a heightened role in promoting phasing. This is an intriguing departure from Drosophila where trailing piRNAs are produced downstream of a site of Piwi protein-initiated cleavage. Here, it seems phasing of piRB-6 can be initiated internally because the region cloned for these triggers only includes an interior section of the locus (FIG. 6D). It is also clear from these results that double-stranded triggers lead to greater production of siRNAs.

### DISCUSSION

This study provides an in-depth analysis of the RNAi pathways in B. tabaci, a hemipteran insect pest and plant virus vector, and offers a rationale design of piRNA-based gene silencing biotechnology. Herein, it is shown that ingested RNAs can enter piRNA pathways, which opens up the possibility for an entirely new strategy for gene silencing and potentially commercial products. On a superficial level, whitefly small RNAs seem similar to Drosophila. There are three distinct types of small RNAs (miRNAs, siRNAs, and piRNAs), as in fruit flies. However, upon close inspection, the biogenesis and function of the endogenous small RNAs in whitefly are quite different. This work reinforces the consistent observation that non-miRNA RNAi pathways are fluid; clade-specific duplication of the RNAi factors is common, even loss of an entire class of small RNA has occurred in several metazoan clades (Sarkies et al, 2015; Calcino et al, 2018; Mondal et al, 2018). Furthermore, these findings illustrate the benefits of in-depth dissection of the RNAi biology for evolutionarily and biologically different organisms, beyond those examined in model study systems, for developing genetic technology.

Through this comprehensive annotation of whitefly small RNA loci, more than 200 novel miRNAs are described, as well as 3,878 siRNA or piRNA loci. Previously described configurations whitefly siRNA and piRNA loci were observed such as large single-stranded, phased piRNA loci and siRNA expressing cis-NAT and hpRNA loci (FIGS. 3 and 8). However, curation of loci found extensive evidence of siRNA and piRNA biogenesis occurring simultaneously at many loci. In fact, this appeared to be the rule for most endogenous siRNA and piRNA genes, and seemingly, is related to a different biogenesis and function for whitefly siRNAs or piRNAs. In Drosophila and vertebrates, piRNAs mainly control TEs in germline; however, many of the piRNA pathway accessory proteins such as Rhino, Deadlock, Cutoff, and Moonshiner from the Drosophilids are not conserved indicating that piRNAs are shaped to individual organism's biology in an evolutionary arm race between the piRNAs and their targets (Ozata et al, 2019). Indeed, it is predicted that abundant somatic piRNAs engage gene regulatory networks in many basal arthropods, such as hemipterans, suggesting that this is the ancestral piRNA biology (Lewis et al, 2018). This combined with observations that whitefly piRNAs respond to viral infection suggest diverse roles for these small RNAs in this insect (Shamimuzzaman et al, 2019). Moreover, it was found that phasing biogenesis can be initiated in the interior of loci as only a portion of the pBias-6 locus as used in the synthetic RNAs. This is distinct from the trigger/responder/trailing piRNA arrangement seen for phased Drosophila piRNAs where a trigger and responder piRNA interaction initiates phasing. In the whitefly system, it appears that antisense small RNAs, perhaps either siRNAs or piRNAs, may be able to slice the transcript and divert it into phasing type biogenesis. Alternatively, a mechanism may be involved where phasing is not triggered by slicing as seen in Drosophila follicle cells (Lau et al, 2009).

Although RNAi has been successful for controlling some pests such as coleopterans (beetles), many other pests such as some lepidopterans (moths and butterflies) are unresponsive to exogenous RNAi trigger (Shukla et al, 2016; Parsons et al, 2018). Penetrance of RNAi in hemipteran insects is moderate, and higher dosage of dsRNA is required (Joga et al, 2016). pH in the gut of the hemipteran insects is basic, and presence of the nucleases in the gut has been reported in whiteflies, aphids, and other hemipteran insects (Luo et al, 2017; Singh et al, 2017). In this study, it was observed that only a minority of the reads produced from the dsRNA trigger are siRNAs (FIGS. 4A and B). This could be attributed to low abundance of the intact dsRNA for uptake by the gut epithelium cells. A similar accumulation of degradation products with piRNA triggers was also seen. Interestingly, even with ssRNA triggers, significant accumulation of small RNA reads from synthetic RNA along with some antisense reads was seen. The antisense reads observed have dominant ping-pong and minor siRNA signature. How the ssRNAs trigger production of these molecules is not clear but could involve the recruitment of target mRNA into small RNA biogenesis. Although this is the standard behavior for piRNAs, it is not typical for siRNAs in organisms that do not possess Rdrp activity (Sarkies et al, 2015; Almeida et al, 2019; Pinzon et al, 2019). This result is therefore viewed as a first glimpse at a heretofore unappreciated small RNA biogenesis mechanism that involves interaction between siRNA and piRNA biogenesis, consistent with the widespread co-occurrence at endogenous loci. This further reinforces the value of knowledge-based RNAi design gleaned from investigating exogenous trigger processing. In this study, methods for maximizing piRNA production are provided, which could be fundamental to potent gene silencing technology aimed at aphids, mealybugs, psyllids, whiteflies, and other hemipterans.

As hemipteran insects respond to exogenous long dsRNA-mediated RNAi trigger only moderately, using the gene silencing function of the piRNA pathway is exciting. These results show that in whitefly, although there is significant sensitivity to dsRNA, there is very little physiological response to dsRNA feeding. Even the secreted gut dsRNases do not become transcriptionally activated by feeding. This will likely apply to other hemipteran herbivores with similar composition of RNAi pathways and dsRNases. piRNA triggers, single-stranded or double-stranded, will likewise likely be physiologically neutral. A promising result reported herein is that exogenous piRNA triggers are as effective as the siRNA versions. This study provides the first report of the exploitation of piRNAs as a feeding-based insect pest control strategy. Thus, this approach could become key for designing effective RNAi approaches against many insect pests that are found to be resistant to dsRNA-mediated RNAi. Finally, dsRNAs are capable of activating interferon response in humans and other vertebrates through binding of TLR3 receptors (Zhang et al, 2016). Deploying ssRNA piRNA triggers as a pest control approach would avoid activating this pathway. As a result, beneficial, non-pest organisms in the field would also be spared from off-target effects of dsRNAs as piRNA triggers rely on the specific genomic sequence of the target species and would not be converted into siRNAs as that happened with dsRNA-based triggers. Taken together, these findings demonstrate the benefit of in-depth studies of non-model organismal RNAi biology and demonstrate that somatic piRNAs can be used for environmental RNAi.

### Example 2

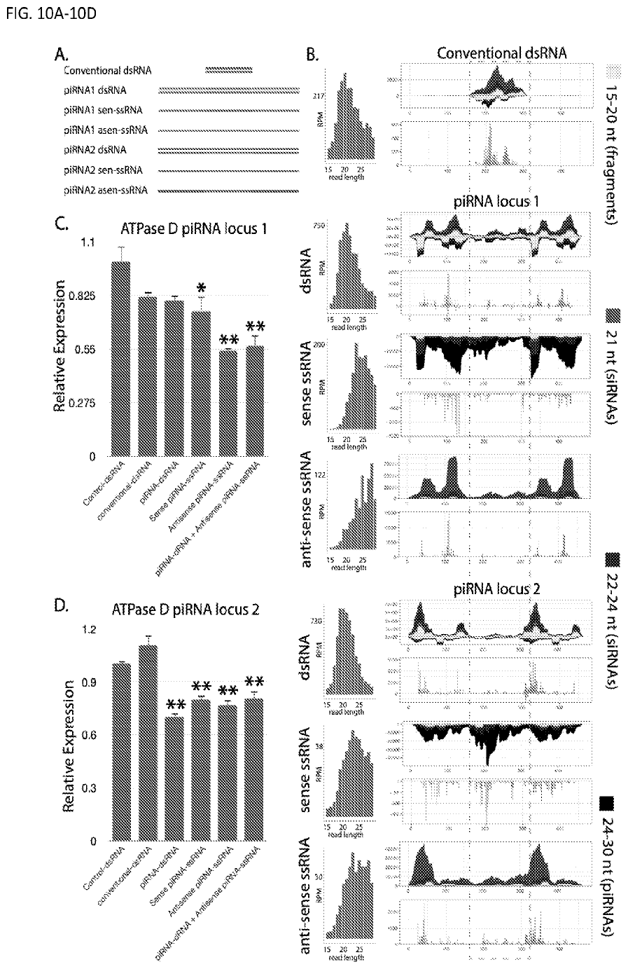

Potato psyllid nymphs were fed seven different synthetic RNAs targeted to v-ATPase-D. The synthetic RNAs used are shown in FIG. 10A. The RNAs against v-ATPase-D were designed around two different piRNA loci and in double-stranded (ds) and single-stranded (ss) configurations. For ssRNAs, antisense and sense were fed separately. The sequences used were as follows:

>P-AF-VD (piRNA 1)
(SEQ ID NO: 75)
AATCTTTCAAATTACCACTAAACTCTTTCAGCTTCAATATTGGAAGTTTGCACTGA
TACTGAGCTATATTACTTCCATTAGCTGATACAAAACTACCATTTTGATCATCTGG
TGTATGTACTGTCTGACTACTCTGAATCTTACTCTCAACCAGATgagaaggcagcttctttcat
gacttcacccatgagggtttttgtctcgatgattttgctcaggatcatacggaatctcatctggagagcatcagctttcttcttaagcaaact
gtgtcccttctgagcccccttgagacgggacttcatgaAATCTTTCAAATTACCACTAAACTCTTTCAGC
TTCAATATTGGAAGTTTGCACTGATACTGAGCTATATTACTTCCATTAGCTGATAC
AAAACTACCATTTTGATCATCTGGTGTATGTACTGTCTGACTACTCTGAATCTTAC
TCTCAACCAGAT
>P-AP-VD (pi RNA 2)
(SEQ ID NO: 76)
TTTCTTCTGAATGTTGAAATGATGTTGATGGAATTGGATTTGACATAGTATCTTCT
GGGCTTGCCTGTATTAGTGGTGTTTTTTGTGCTTCTTTATAGGGCTTTCTTTTCTTG
GCAGGGGGTTGTAAGTAAGAGGGAAAAGCTGTAAAAACTGAAGGAAgagaaggcagc
ttctttcatgacttcacccatgagggtttttgtctcgatgattttgctcaggatcatacggaatctcatctggagagcatcagctttcttcttaa
gcaaactgtgtcccttctgagcccccttgagacgggacttcatgaTTTCTTCTGAATGTTGAAATGATGTTGA
TGGAATTGGATTTGACATAGTATCTTCTGGGCTTGCCTGTATTAGTGGTGTTTTTT
GTGCTTCTTTATAGGGCTTTCTTTTCTTGGCAGGGGGTTGTAAGTAAGAGGGAAA
AGCTGTAAAAACTGAAGGAA

Sequences in lowercase are the target sequences, and the left and right blanking sequences are shown in capital letters.

After feeding conventional dsRNA to the nymphs, small RNAs were sequenced. Results are shown in FIG. 10B. The graph on the left shows distribution of reads that align. Consistent with siRNA generation, reads were biased to 20-23 nt. The top right graph shows read mapping densities. Anti-sense strand mapping reads are positive y-axis values and sense strand mapping reads are negative y-axis values. Color in the plot indicates the sizes of reads as indicated on the right of the diagram. The bottom right plot shows the location of >24 nt reads that show evidence of piRNA-phasing type biogenesis. This characterized by 5′ terminal U-base and being directly downstream of another U-base read. The red dashed box indicates region that corresponds to the vATPase D complementary region.

FIG. 10C shows the efficacy of piRNA locus 1 RNAs. The bar chart on the left shows the ability of the piRNA 1 RNAs (shown in FIG. 10A) to suppress target gene expression. (*p<0.05, **p<0.01). Feeding ssRNAs led to greater suppression of gene expression. The graphs on the right show the same information as shown in FIG. 10B. Namely, the graphs on the left show the distribution of reads that align for dsRNA, sense ssRNA, and anti-sense ssRNA groups. The graphs on the right show read mapping densities and the plots show the location of >24 nt reads that show evidence of piRNA-phasing type biogenesis. The color in the graphs indicates the sizes of reads as indicated on the right of the diagram. Here for ssRNAs larger, piRNA-sized RNAs are found. Phasing is more evident for sense RNAs, consistent with trigger piRNAs initiating this process. However, piRNAs are seen also with antisense, which would be complementary to v-ATPase-D, and lead to greater suppression of this mRNA. However, all ssRNAs were superior to dsRNAs for gene silencing.

FIG. 10 D shows efficiency of piRNA locus 2 RNAs. The bar chart on the left shows the ability of piRNAs to suppress target gene expression. The graphs on the right show the same information as shown in FIG. 10B and FIG. 10C. Namely, the graphs on the left show the distribution of reads that align for dsRNA, sense ssRNA, and anti-sense ssRNA groups. The graphs on the right show read mapping densities and the plots show the location of >24 nt reads that show evidence of piRNA-phasing type biogenesis. The color in the graphs indicates the sizes of reads as indicated on the right of the diagram. Here similar results are seen with the exception that dsRNA configured piRNA locus shows knockdown. This may be due to the phasing piRNAs that are seen more broadly relative to locus 1. The difference in performance of piRNA locus 1 vs piRNA locus 2 highlights the variability of piRNA biogenesis and the importance of evaluating locus features in designing effective RNAi approaches.

### REFERENCES

Almeida M V, Andrade-Navarro M A, Ketting R F (2019) Function and evolution of nematode RNAi pathways. Noncoding RNA 5: 8. doi:10.3390/ncma5010008
Antoniewski C (2014) Computing siRNA and piRNA overlap signatures. Methods Mol Biol 1173: 135-146. doi:10.1007/978-1-4939-0931-5_12
Bartel D P (2018) Metazoan microRNAs. Cell 173: 20-51. doi:10.1016/j.cell.2018.03.006
Berezikov E, Liu N, Flynt A S, Hodges E, Rooks M, Hannon G J, Lai E C (2010) Evolutionary flux of canonical microRNAs and mirtrons in Drosophila. Nat Genet 42: 6-9. doi:10.1038/ng0110-6
Brito T, Julio A, Beri M, de Castro Poncio L, Bernardes E S, Araujo H, Sammeth M, Pane A (2018) Transcriptomic and functional analyses of the piRNA pathway in the Chagas disease vector Rhodnius prolixus. PLoS Negl Trop Dis 12: e0006760. doi:10.1371/joumal.pntd.0006760
Brown J K, Frohlich D R, Rosell R C (1995) The sweetpotato or silverleaf whiteflies: Biotypes of Bemisia tabaci or a species complex?. Annu Rev Entomol 40: 511-534. doi:10.1 146/annurev.en.40.010195.002455
Brown J K (2010) Phylogenetic biology of the Bemisia tabaci sibling species group. In Bemisia: Bionomics and Management of a Global Pest. Stansly P A, Naranjo S E (eds.). pp 31-67. Dordrecht: Springer Netherlands.
Calcino A D, Fernandez-Valverde S L, Taft R J, Degnan B M (2018) Diverse RNA interference strategies in early-branching metazoans. BMC Evol Biol 18: 160. doi:10.1186/s12862-018-1274-2
Carthew R W, Sontheimer E J (2009) Origins and mechanisms of miRNAs and siRNAs. Cell 136: 642-655. doi:10.1016/j.cell.2009.01.035
Chen W, Hasegawa D K, Kaur N, Kliot A, Pinheiro P V, Luan J, Stensmyr M C, Zheng Y, Liu W, Sun H, et al (2016) The draft genome of whitefly Bemisia tabaci MEAM1, a global crop pest, provides novel insights into virus transmission, host adaptation, and insecticide resistance. BMC Biol 14: 110. doi:10.1186/s12915-016-0321-y
Cicero J M, Brown J K (2011) Functional anatomy of whitefly organs associated with squash leaf curl virus (Geminiviridae: Begomovirus) transmission by the B biotype of Bemisia tabaci (Hemiptera: Aleyrodidae). Ann Entomol Soc America 104: 261-279. doi:10.1603/an10075
Claycomb J M (2014) Ancient endo-siRNA pathways reveal new tricks. Curr Biol 24: R703-R715. doi:10.1016/j.cub.2014.06.009
Cristofoletti P T, Ribeiro A F, Deraison C, Rahbe Y, Terra W R (2003) Midgut adaptation and digestive enzyme distribution in a phloem feeding insect, the pea aphid Acyrthosiphon pisum. J Insect Physiol 49: 11-24. doi:10.1016/s0022-1910(02)00222-6
Crooks G E, Hon G, Chandonia J-M, Brenner S E (2004) WebLogo: A sequence logo generator. Genome Res 14: 1188-1190. doi: 10.1101/gr.849004
Czech B, Malone C D, Zhou R, Stark A, Schlingeheyde C, Dus M, Perrimon N, Kellis M, Wohlschlegel J A, Sachidanandam R, et al (2008) An endogenous small interfering RNA pathway in Drosophila. Nature 453: 798-802. doi:10.1038/nature07007
de Moya R S, Brown J K, Sweet A D, Walden K K O, Paredes-Montero J R, Waterhouse R M, Johnson K P (2019) Nuclear orthologs derived from whole genome sequencing indicate cryptic diversity in the Bemisia tabaci (insecta: Aleyrodidae) complex of whiteflies. Diversity 11: 151. doi:10.3390/d11090151
de Paula N T, de Faria J C, Aragao F J (2015) Reduction of viral load in whitefly (Bemisia tabaci Gen.) feeding on RNAi-mediated bean golden mosaic virus resistant transgenic bean plants. Virus Res 210: 245-247. doi:10.1016/j.virusres.2015.08.012
El-Shesheny I, Hajeri S, El-Hawary I, Gowda S, Killiny N (2013) Silencing abnormal wing disc gene of the Asian citrus psyllid, Diaphorina citri disrupts adult wing development and increases nymph mortality. PLoS One 8: e65392. doi:10.1371/journal.pone.0065392
Fagegaltier D, Bouge A-L, Berry B, Poisot E, Sismeiro O, Coppee J-Y, Theodore L, Voinnet O, Antoniewski C (2009) The endogenous siRNA pathway is involved in heterochromatin formation in Drosophila. Proc Natl Acad Sci USA 106: 21258-21263. doi:10.1073/pnas.0809208105
Friedlander M R, Chen W, Adamidi C, Maaskola J, Einspanier R, Knespel S, Rajewsky N (2008) Discovering microRNAs from deep sequencing data using miRDeep. Nat Biotechnol 26: 407-415. doi:10.1038/nbt1394
Friedlander M R, Mackowiak S D, Li N, Chen W, Rajewsky N (2012) miRDeep2 accurately identifies known and hundreds of novel microRNA genes in seven animal clades. Nucleic Acids Res 40: 37-52. doi:10.1093/nar/gkr688
Gainetdinov I, Colpan C, Arif A, Cecchini K, Zamore P D (2018) A single mechanism of biogenesis, initiated and directed by PIWI proteins, explains piRNA production in most animals. Mol Cell 71: 775-790.e5. doi:10.1016/j.molcel.2018.08.007
Ghildiyal M, Seitz H, Horwich M D, Li C, Du T, Lee S, Xu J, Kittler E L, Zapp M L, Weng Z, et al (2008) Endogenous siRNAs derived from transposons and mRNAs in Drosophila somatic cells. Science 320: 1077-1081. doi:10.1126/science.1157396
Grover S, Jindal V, Banta G, Taning C N T, Smagghe G, Christiaens O (2019) Potential of RNA interference in the study and management of the whitefly, Bemisia tabaci. Arch Insect Biochem Physiol 100: e21522. doi:10.1002/arch.21522
Han B W, Wang W, Zamore P D, Weng Z (2015) piPipes: A set of pipelines for piRNA and transposon analysis via small RNA-seq, RNA-seq, degradome- and CAGE-seq, ChIP-seq and genomic DNA sequencing. Bioinformatics 31: 593-595. doi:10.1093/bioinformatics/btu647
Head G P, Carroll M W, Evans S P, Rule D M, Willse A R, Clark T L, Storer N P, Flannagan R D, Samuel L W, Meinke L J (2017) Evaluation of SmartStax and SmartStax PRO maize against western corn rootworm and northern corn rootworm: Efficacy and resistance management. Pest Manag Sci 73: 1883-1899. doi:10.1002/ps.4554
Huang X, Toth K F, Aravin A A (2017) piRNA biogenesis in Drosophila melanogaster. Trends Genet 33: 882-894. doi:10.1016/j.tig.2017.09.002 Ishizu H, Kinoshita T, Hirakata S,
Komatsuzaki C, Siomi M C (2019) Distinct and collaborative functions of Yb and armitage in transposon-targeting piRNA biogenesis. Cell Rep 27: 1822-1835.e8. doi:10.1016/j.celrep.2019.04.029
Jaubert-Possamai S, Le Trionnaire G, Bonhomme J, Christophides G K, Rispe C, Tagu D (2007) Gene knockdown by RNAi in the pea aphid Acyrthosiphon pisum. BMC Biotechnol 7: 63. doi:10.1186/1472-6750-7-63
Jelinek I, Leonard J N, Price G E, Brown K N, Meyer-Manlapat A, Goldsmith P K, Wang Y, Venzon D, Epstein S L, Segal D M (2011) TLR3-specific double-stranded RNA oligonucleotide adjuvants induce dendritic cell crosspresentation, CTL responses, and antiviral protection. J Immunol 186: 2422-2429. doi:10.4049/jimmunol.1002845
Joga M R, Zotti M J, Smagghe G, Christiaens O (2016) RNAi efficiency, systemic properties, and novel delivery methods for pest insect control: What we know so far. Front Physiol 7: 553. doi:10.3389/fphys.2016.00553
Facile piRNA-mediated gene silencing in the whitefly Mondal et al. https://doi.org/10.26508/lsa.202000731 vol 3|no 10|e202000731 12 of 13
Kanakala S, Kontsedalov S, Lebedev G, Ghanim M (2019) Plant-mediated silencing of the whitefly Bemisia tabaci cyclophilin B and heat shock protein 70 impairs insect development and virus transmission. Front Physiol 10: 557. doi:10.3389/fphys.2019.00557
Knorr E, Fishilevich E, Tenbusch L, Frey M L F, Rangasamy M, Billion A, Worden S E, Gandra P, Arora K, Lo W, et al (2018) Gene silencing in Tribolium castaneum as a tool for the targeted identification of candidate RNAi targets in crop pests. Sci Rep 8: 2061. doi:10.1038/s41598-018-20416-y
Kolde R (2012) Pheatmap: Pretty heatmaps. R Package Version 61: 915. Kozomara A, Birgaoanu M, Griffiths-Jones S (2019) miRBase: From microRNA sequences to function. Nucleic Acids Res 47: D155-D162. doi:10.1093/nar/gky1141
Langdon W B (2015) Performance of genetic programming optimised Bowtie2 on genome comparison and analytic testing (GCAT) benchmarks. BioData Min 8: 1. doi:10.1186/s13040-014-0034-0
Lau N C, Robine N, Martin R, Chung W J, Niki Y, Berezikov E, Lai E C (2009) Abundant primary piRNAs, endo-siRNAs, and microRNAs in a Drosophila ovary cell line. Genome Res 19: 1776-1785. doi:10.1101/gr.094896.109
Lewis S H, Quarles K A, Yang Y, Tanguy M, Frezal L, Smith S A, Sharma P P, Cordaux R, Gilbert C, Giraud I, et al (2018) Pan-arthropod analysis reveals somatic piRNAs as an ancestral defence against transposable elements. Nat Ecol Evol 2: 174-181. doi:10.1038/s41559-017-0403-4
Li X, Zhang F, Coates B, Zhang Y, Zhou X, Cheng D (2016) Comparative profiling of microRNAs in the winged and wingless English grain aphid, Sitobion avenae (F.) (Homoptera: Aphididae). Sci Rep 6: 35668. doi:10.1038/srep35668
Ligges U, Maechler M, Schnackenberg S, Ligges M U (2018) Package “scatterplot3d”. Recuperado de https://cran.rproject.org/web/packages/scatterplot3d/scatterplot3d.pdf.
Luo Y, Chen Q, Luan J, Chung S H, Van Eck J, Turgeon R, Douglas A E (2017) Towards an understanding of the molecular basis of effective RNAi against a global insect pest, the whitefly Bemisia tabaci. Insect Biochem Mol Biol 88: 21-29. doi:10.1016/j.ibmb.2017.07.005
Malik H J, Raza A, Amin I, Scheffler J A, Scheffler B E, Brown J K, Mansoor S (2016) RNAi-mediated mortality of the whitefly through transgenic expression of double-stranded RNA homologous to acetylcholinesterase and ecdysone receptor in tobacco plants. Sci Rep 6: 38469. doi:10.1038/srep38469
Molki B, Ha P T, Cohen A L, Crowder D W, Gang D R, Omsland A, Brown J K, Beyenal H (2019) The infection of its insect vector by bacterial plant pathogen “Candidatus Liberibacter solanacearum” is associated with altered vector physiology. Enzyme Microb Technol 129: 109358. doi:10.1016/j.enzmictec.2019.109358
Mondal M, Klimov P, Flynt A S (2018) Rewired RNAi-mediated genome surveillance in house dust mites. PLoS Genet 14: e1007183. doi:10.1371/journal.pgen.1007183
Okamura K (2012) Diversity of animal small RNA pathways and their biological utility. Wiley Interdiscip Rev RNA 3: 351-368. doi:10.1002/wrna.113
Ozata D M, Gainetdinov I, Zoch A, O'Carroll D, Zamore P D (2019) PIWI-interacting RNAs: Small RNAs with big functions. Nat Rev Genet 20:89-108. doi:10.1038/s41576-018-0073-3
Parsons K H, Mondal M H, McCormick C L, Flynt A S (2018) Guanidinium-functionalized interpolyelectrolyte complexes enabling RNAi in resistant insect pests. Biomacromolecules 19: 1111-1117. doi:10.1021/acs.biomac.7b01717
Phanstiel D H, Boyle A P, Araya C L, Snyder M P (2014) Sushi.R: Flexible, quantitative and integrative genomic visualizations for publication-quality multi-panel figures. Bioinformatics 30: 2808-2810. doi:10.1093/bioinformatics/btu379 Pinzon N, Bertrand S, Subirana L, Busseau I, Escriva H, Seitz H (2019)
Functional lability of RNA-dependent RNA polymerases in animals. PLoS Genet 15: e1007915. doi:10.1371/journal.pgen.1007915 Quinlan A R, Hall I M (2010) BEDTools: A flexible suite of utilities for comparing genomic features. Bioinformatics 26: 841-842. doi:10.1093/bioinformatics/btq033
Sarkies P, Selkirk M E, Jones J T, Blok V, Boothby T, Goldstein B, Hanelt B, Ardila-Garcia A, Fast N M, Schiffer P M, et al (2015) Ancient and novel small RNA pathways compensate for the loss of piRNAs in multiple independent nematode lineages. PLoS Biol 13: e1002061. doi:10.1371/joumal.pbio.1002061
Shamimuzzaman M, Hasegawa D K, Chen W, Simmons A M, Fei Z, Ling K S (2019) Genome-wide profiling of piRNAs in the whitefly Bemisia tabaci reveals cluster distribution and association with begomovirus transmission. PLoS One 14: e0213149. doi:10.1371/journal.pone.0213149
Shukla J N, Kalsi M, Sethi A, Narva K E, Fishilevich E, Singh S, Mogilicherla K, Palli S R (2016) Reduced stability and intracellular transport of dsRNA contribute to poor RNAi response in lepidopteran insects. RNA Biol 13:656-669. doi:10.1080/15476286.2016.1191728
Singh I K, Singh S, Mogilicherla K, Shukla J N, Palli S R (2017) Comparative analysis of double-stranded RNA degradation and processing in insects. Sci Rep 7: 17059. doi:10.1038/s41598-017-17134-2
Thakur N, Upadhyay S K, Verma P C, Chandrashekar K, Tuli R, Singh P K (2014) Enhanced whitefly resistance in transgenic tobacco plants expressing double stranded RNA of v-ATPase A gene. PLoS One 9: e87235. doi:10.1371/joumal.pone.0087235
Vyas M, Raza A, Ali M Y, Ashraf M A, Mansoor S, Shahid A A, Brown J K (2017) Knock down of whitefly gut gene expression and mortality by orally delivered gut gene-specific dsRNAs. PLoS One 12: e0168921. doi:10.1371/journal.pone.0168921
Wang B, Wang L, Chen F, Yang X, Ding M, Zhang Z, Liu S S, Wang X W, Zhou X (2016) MicroRNA profiling of the whitefly Bemisia tabaci Middle East-Aisa Minor I following the acquisition of Tomato yellow leaf curl China virus. Virol J 13: 20. doi:10.1186/s12985-016-0469-7
Wames M G R, Bolker B, Bonebakker L, Gentleman R, Huber W (2016) Package “gplots.” Various R programming tools for plotting data
Wickham H (2016) ggplot2: Elegant Graphics for Data Analysis. New York: Springer-Verlag.
Yamaguchi S, Oe A, Nishida K M, Yamashita K, Kajiya A, Hirano S, Matsumoto N, Dohmae N, Ishitani R, Saito K, et al (2020) Crystal structure of Drosophila Piwi. Nat Commun 11: 858. doi:10.1038/s41467-020-14687-1
Yamanaka S, Siomi M C, Siomi H (2014) piRNA clusters and open chromatin structure. Mob DNA 5: 22. doi:10.1186/1759-8753-5-22
Yu N, Christiaens O, Liu J, Niu J, Cappelle K, Caccia S, Huvenne H, Smagghe G (2013) Delivery of dsRNA for RNAi in insects: An overview and future directions. Insect Sci 20: 4-14. doi:10.1111/j.1744-7917.2012.01534.x
Zha W, Peng X, Chen R, Du B, Zhu L, He G (2011) Knockdown of midgut genes by dsRNA-transgenic plant-mediated RNA interference in the hemipteran insect Nilaparvata lugens. PLoS One 6: e20504. doi:10.1371/journal.pone.0020504
Zhang L, Xiang W, Wang G, Yan Z, Zhu Z, Guo Z, Sengupta R, Chen A F, Loughran P A, Lu B, et al (2016) Interferon beta (IFN-beta) production during the double-stranded RNA (dsRNA) response in hepatocytes involves coordinated and feedforward signaling through Toll-like receptor 3 (TLR3), RNA-dependent protein kinase (PKR), inducible nitric oxide synthase (iNOS), and Src protein. J Biol Chem 291: 15093-15107. doi:10.1074/jbc.m116.717942
Zhu K Y, Palli S R (2020) Mechanisms, applications, and challenges of insect RNA interference. Annu Rev Entomol 65: 293-311. doi:10.1146/annurev-ento-011019-025224
Zotti M J, Smagghe G (2015) RNAi technology for insect management and protection of beneficial insects from diseases: Lessons, challenges and risk assessments. Neotrop Entomol 44: 197-213. doi:10.1007/s13744-015-0291-8

All publications, patents, patent applications and accession numbers mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. A synthetic RNA comprising:
a. A piRNA sequence; and
b. A target sequence for a gene of interest,
wherein the synthetic RNA induces silencing of the gene of interest.

2. The synthetic RNA of claim 1, wherein the synthetic RNA is single-stranded.

3. The synthetic RNA of claim 1, wherein the synthetic RNA is double-stranded.

4. The synthetic RNA of any one of claims 1-3, wherein the piRNA sequence comprises a left flanking sequence and a right flanking sequence, and wherein the target sequence for the gene of interest is sandwiched in between the left flanking sequence and the right flanking sequence.

5. The synthetic RNA of any one of the preceding claims, wherein the gene of interest is a gene in a hemipteran organism.

6. The synthetic RNA of any one of claims 1-5, wherein the gene of interest is aquaporin (AQP1), alpha glucosidase 1 (AGLU1), v-ATPase-A, v-ATPase-B, v-ATPase-D, v-ATPase-E, Delta-24 sterol reductase (D-24), cholesterol desaturase (C7), Cryptocephal (Crc), Chitinase 7, Chitinase 5, Chitin Synthase, Endochitinase, Coractin, Actin, Wiskott-Aldrich syndrome protein (WASP), Rac Family Small GTPase 1 (RAC1), BAR/IMD Domain Containing Adaptor Protein 2 (IRSp53), WASP-family verprolin-homologous protein (WAVE), or Actin related 2/3.

7. The synthetic RNA of claim 6, wherein the target sequence comprises at least 20 contiguous nucleotides present in the gene of interest.

8. The synthetic RNA of claim 7, wherein the target sequence comprises at least 20 contiguous nucleotides having at least 80% sequence identity with at least 20 contiguous nucleotides present in SEQ ID NO: 2, SEQ ID NO: 3, SEQ ID NO: 70, or any one of SEQ ID NO: 14-47.

9. The synthetic RNA of any one of the preceding claims, wherein silencing of the gene of interest is induced at least in part by ping-pong biogenesis of piRNAs.

10. The synthetic RNA of any one of the preceding claims, wherein silencing of the gene of interest is induced at least in part by biogenesis of piRNAs through piRNA phasing.

11. The synthetic RNA of any one of claims 4-10, wherein the left flanking region comprises a nucleotide sequence having at least 80% sequence identity with SEQ ID NO: 4 and the right flanking region comprises a nucleotide sequence having at least 80% sequence identity with SEQ ID NO: 5.

12. The synthetic RNA of claim 11, wherein the left flanking region comprises a nucleotide sequence having at least 90% sequence identity with SEQ ID NO: 4 and the right flanking region comprises a nucleotide sequence having at least 90% sequence identity with SEQ ID NO: 5.

13. The synthetic RNA of claim 12, wherein the left flanking region comprises the nucleotide sequence of SEQ ID NO: 4 and the right flanking region comprises the nucleotide sequence of SEQ ID NO: 5.

14. The synthetic RNA of any one of claims 4-10, wherein the left flanking region comprises a nucleotide sequence having at least 80% sequence identity with SEQ ID NO: 6 and the right flanking region comprises a nucleotide sequence having at least 80% sequence identity with SEQ ID NO: 7.

15. The synthetic RNA of claim 14, wherein the left flanking region comprises a nucleotide sequence having at least 90% sequence identity with SEQ ID NO: 6 and the right flanking region comprises a nucleotide sequence having at least 90% sequence identity with SEQ ID NO: 7.

16. The synthetic RNA of claim 15, wherein the left flanking region comprises the nucleotide sequence of SEQ ID NO: 6 and the right flanking region comprises the nucleotide sequence of SEQ ID NO: 7.

17. The synthetic RNA of any one of claims 11-16, wherein the gene of interest is aquaporin (AQP1) or alpha glucosidase 1 (AGLU1).

18. The synthetic RNA of claim 17, wherein the target sequence comprises at least 20 contiguous nucleotides having at least 80% sequence identity with at least 20 contiguous nucleotides present in SEQ ID NO: 2 or SEQ ID NO: 3.

19. The synthetic RNA of claim 18, wherein the target sequence comprises SEQ ID NO: 2 or SEQ ID NO: 3.

20. The synthetic RNA of any one of the preceding claims, comprising a nucleotide sequence having at least 80% sequence identity with SEQ ID NO: 8 or SEQ ID NO: 9.

21. The synthetic RNA of claim 20, for use in a method of silencing expression of AQP1 in a hemipteran organism.

22. The synthetic RNA of claim 21, wherein the hemipteran organism is a whitefly.

23. The synthetic RNA of any one of claims 1-19, comprising a nucleotide sequence having at least 80% sequence identity with SEQ ID NO: 10 or SEQ ID NO: 11.

24. The synthetic RNA of claim 23, for use in a method of silencing expression of AGLU1 in a hemipteran organism.

25. The synthetic RNA of claim 24, wherein the hemipteran organism is a whitefly.

26. The synthetic RNA of any one of claims 4-10, wherein the left flanking region comprises a nucleotide sequence having at least 80% sequence identity with SEQ ID NO: 71 and the right flanking region comprises a nucleotide sequence having at least 80% sequence identity with SEQ ID NO: 72.

27. The synthetic RNA of claim 26, wherein the left flanking region comprises a nucleotide sequence having at least 90% sequence identity with SEQ ID NO: 71 and the right flanking region comprises a nucleotide sequence having at least 90% sequence identity with SEQ ID NO: 72.

28. The synthetic RNA of claim 27, wherein the left flanking region comprises the nucleotide sequence of SEQ ID NO: 71 and the right flanking region comprises the nucleotide of SEQ ID NO: 72.

29. The synthetic RNA of any one of claims 4-10, wherein the left flanking region comprises a nucleotide sequence having at least 80% sequence identity with SEQ ID NO: 73 and the right flanking region comprises a nucleotide sequence having at least 80% sequence identity with SEQ ID NO: 74.

30. The synthetic RNA of claim 29, wherein the left flanking region comprises a nucleotide sequence having at least 90% sequence identity with SEQ ID NO: 73 and the right flanking region comprises a nucleotide sequence having at least 90% sequence identity with SEQ ID NO: 74.

31. The synthetic RNA of claim 30, wherein the left flanking region comprises the nucleotide sequence of SEQ ID NO: 73 and the right flanking region comprises the nucleotide of SEQ ID NO: 74.

32. The synthetic RNA of any one of claims 26-31, wherein the gene of interest is v-ATPase-D.

33. The synthetic RNA of claim 32, wherein the target sequence comprises at least 20 contiguous nucleotides having at least 80% sequence identity with at least 20 contiguous nucleotides present in SEQ ID NO: 70.

34. The synthetic RNA of claim 33, wherein the target sequence comprises SEQ ID NO: 70.

35. The synthetic RNA of any one of claims 26-34, comprising a nucleotide sequence having at least 80% sequence identity to SEQ ID NO: 75 or SEQ ID NO: 76.

36. The synthetic RNA of claim 34, for use in a method of silencing expression of v-ATPase-D in a hemipteran organism.

37. The synthetic RNA of claim 36, wherein the hemipteran organism is a potato psyllid.

38. The synthetic RNA of any one of the preceding claims, for use in a method of silencing gene expression in an organism.

39. The synthetic RNA of claim 38, wherein the organism is a hemipteran.

40. A method of silencing gene expression in an organism, comprising providing to the organism the synthetic RNA of any one of the preceding claims.

41. The method of claim 40, wherein the organism is a hemipteran.


SEQUENCE LISTING (76 sequences)

SEQ ID NO: 1 - protein, 262 aa, synthetic construct
MEDISSSGEEISMKAISKVIGVPDIRDGPTLTKCIVAEFVGTLLLVLIGCMSVAFVHQDNFVDVVKIAMAFGLIIASMVQAIGHVSGCHINPAVTCGLAVSGHVSIIKGMLYIVAQCLGAICGAIILNEITPKTGYTAAGNLGVTTLSTGVSDLQGVAIEALITFVLLLVVQSVCDGKRTDIKGSIGVAIGFAIACCHLAAIKYTGASMNPARSLGPAFVSGIWDKHWVYWAGPILGGVTASLLYAITFKAKKRSDESSYDF

SEQ ID NO: 2 - RNA, 245 nt, synthetic construct
  source 1..245: mol_type = other RNA
tcgcacaatgccttggagccatctgtggagcaatcattctgaatgaaatcacgccaaaaacaggttacacggctgctggtaatctgggagtaacgacactgtctacaggagtttccgacctgcagggtgtggcgatagaagcactaatcacatttgtgctgcttttagttgtccagtccgtctgcgatgggaagcggaccgacatcaaaggatctatcggcgttgcgataggattcgcaattgct

SEQ ID NO: 3 - RNA, 325 nt, synthetic construct
  source 1..325: mol_type = other RNA
ctgtccatccaaccctggattgccttttggtaatctttggcgggagagcgaccgctcacgtgcgtaataaagaagaaattgaatggcatatgggctcctggtttcccctcaaactgataatagtccattgttctatcgagagttgtatatgcttctgtcattagtacttttgttttcccctctttctttgtgtagaaatcgaagacctccctgaaccttgttatcaatctatatgtattcggttggtccatcgtccgcgaccggttgtaattccagtagttcgtcgggtcgagatcgggcgagagcaattcttggtctcgccatt

SEQ ID NO: 4 - RNA, 342 nt, synthetic construct
  source 1..342: mol_type = other RNA
agcagcttcttgcctctgattccacggtttcttcttaaagggccccgacgactgctgcgggccttgataaggcgcgctcctgttatttgcctcacggaacgtcttttccgcggccatcattgcgtccattgatcggatcaaatcttgcctcattgcatccacggctcgagtattcctatccgtatccgcacgatttagatcaactgcgtgtaccaaagtcgctagggcgttctcattggccttcacccgggattctaaggatgattcctgccccgtatagtgatttacggccaaaatagcgccccttcctttgctggtcgcggctactgctagcttcgcatt

SEQ ID NO: 5 - RNA, 366 nt, synthetic construct
  source 1..366: mol_type = other RNA
tccgtcgagttaactttagccaagcccgctagttttctcttcgcttgaacgtaatccaacgggtcctcattttctccctgcgttcgcgccgagaatttcgtgagggcatcctcgtcgctgtcaaagtattggatcaatttcttctttacttcctcaaaagtcctgcagttaccgaacgctacctcttcattgtcgtagtactggatggcacgtttcgctaagtgatttctgagttggtcccgcttttcttgatccgaacatttcttatagaaattttcaaaatcttttagaaattctctaacgtcgtagtcagcttctcctttgaatagttttctaaacggcggtgccttaatcgtcaccgtaggt

SEQ ID NO: 6 - RNA, 238 nt, synthetic construct
  source 1..238: mol_type = other RNA
ttgcgttcctgctccctttgccctttaccgcgctcaattatctctattagaaccggagatattcggtttacaaaaattttttggggcccagccccccttaatcctttccctatggacttcctatatggccccagaggtagcccccgggggttaggcaaataatcccaaaaaattcccaaattctaacggaaatgtggcactaccgcccctacgtcactctggctatgacgtagttgat

SEQ ID NO: 7 - RNA, 199 nt, synthetic construct
  source 1..199: mol_type = other RNA
ttacgtgccgttacaccggttaccgacatcaggttccttcaaatcggacacgggcgcccctccccgaggggatgccaatggggggaggtcccaggccgaagcctgactttctactacctccggagctgtgcccttctctgcacgtcccagttgagcactggtgggctgacctcggggacaaggtcgccttaacttaccg

SEQ ID NO: 8 - RNA, 953 nt, synthetic construct
  source 1..953: mol_type = other RNA
agcagcttcttgcctctgattccacggtttcttcttaaagggccccgacgactgctgcgggccttgataaggcgcgctcctgttatttgcctcacggaacgtcttttccgcggccatcattgcgtccattgatcggatcaaatcttgcctcattgcatccacggctcgagtattcctatccgtatccgcacgatttagatcaactgcgtgtaccaaagtcgctagggcgttctcattggccttcacccgggattctaaggatgattcctgccccgtatagtgatttacggccaaaatagcgccccttcctttgctggtcgcggctactgctagcttcgcatttcgcacaatgccttggagccatctgtggagcaatcattctgaatgaaatcacgccaaaaacaggttacacggctgctggtaatctgggagtaacgacactgtctacaggagtttccgacctgcagggtgtggcgatagaagcactaatcacatttgtgctgcttttagttgtccagtccgtctgcgatgggaagcggaccgacatcaaaggatctatcggcgttgcgataggattcgcaattgcttccgtcgagttaactttagccaagcccgctagttttctcttcgcttgaacgtaatccaacgggtcctcattttctccctgcgttcgcgccgagaatttcgtgagggcatcctcgtcgctgtcaaagtattggatcaatttcttctttacttcctcaaaagtcctgcagttaccgaacgctacctcttcattgtcgtagtactggatggcacgtttcgctaagtgatttctgagttggtcccgcttttcttgatccgaacatttcttatagaaattttcaaaatcttttagaaattctctaacgtcgtagtcagcttctcctttgaatagttttctaaacggcggtgccttaatcgtcaccgtaggt

SEQ ID NO: 9 - RNA, 682 nt, synthetic construct
  source 1..682: mol_type = other RNA
ttgcgttcctgctccctttgccctttaccgcgctcaattatctctattagaaccggagatattcggtttacaaaaattttttggggcccagccccccttaatcctttccctatggacttcctatatggccccagaggtagcccccgggggttaggcaaataatcccaaaaaattcccaaattctaacggaaatgtggcactaccgcccctacgtcactctggctatgacgtagttgattcgcacaatgccttggagccatctgtggagcaatcattctgaatgaaatcacgccaaaaacaggttacacggctgctggtaatctgggagtaacgacactgtctacaggagtttccgacctgcagggtgtggcgatagaagcactaatcacatttgtgctgcttttagttgtccagtccgtctgcgatgggaagcggaccgacatcaaaggatctatcggcgttgcgataggattcgcaattgctttacgtgccgttacaccggttaccgacatcaggttccttcaaatcggacacgggcgcccctccccgaggggatgccaatggggggaggtcccaggccgaagcctgactttctactacctccggagctgtgcccttctctgcacgtcccagttgagcactggtgggctgacctcggggacaaggtcgccttaacttaccg

SEQ ID NO: 10 - RNA, 1033 nt, synthetic construct
  source 1..1033: mol_type = other RNA
agcagcttcttgcctctgattccacggtttcttcttaaagggccccgacgactgctgcgggccttgataaggcgcgctcctgttatttgcctcacggaacgtcttttccgcggccatcattgcgtccattgatcggatcaaatcttgcctcattgcatccacggctcgagtattcctatccgtatccgcacgatttagatcaactgcgtgtaccaaagtcgctagggcgttctcattggccttcacccgggattctaaggatgattcctgccccgtatagtgatttacggccaaaatagcgccccttcctttgctggtcgcggctactgctagcttcgcattctgtccatccaaccctggattgccttttggtaatctttggcgggagagcgaccgctcacgtgcgtaataaagaagaaattgaatggcatatgggctcctggtttcccctcaaactgataatagtccattgttctatcgagagttgtatatgcttctgtcattagtacttttgttttcccctctttctttgtgtagaaatcgaagacctccctgaaccttgttatcaatctatatgtattcggttggtccatcgtccgcgaccggttgtaattccagtagttcgtcgggtcgagatcgggcgagagcaattcttggtctcgccatttccgtcgagttaactttagccaagcccgctagttttctcttcgcttgaacgtaatccaacgggtcctcattttctccctgcgttcgcgccgagaatttcgtgagggcatcctcgtcgctgtcaaagtattggatcaatttcttctttacttcctcaaaagtcctgcagttaccgaacgctacctcttcattgtcgtagtactggatggcacgtttcgctaagtgatttctgagttggtcccgcttttcttgatccgaacatttcttatagaaattttcaaaatcttttagaaattctctaacgtcgtagtcagcttctcctttgaatagttttctaaacggcggtgccttaatcgtcaccgtaggt

SEQ ID NO: 11 - RNA, 762 nt, synthetic construct
  source 1..762: mol_type = other RNA
ttgcgttcctgctccctttgccctttaccgcgctcaattatctctattagaaccggagatattcggtttacaaaaattttttggggcccagccccccttaatcctttccctatggacttcctatatggccccagaggtagcccccgggggttaggcaaataatcccaaaaaattcccaaattctaacggaaatgtggcactaccgcccctacgtcactctggctatgacgtagttgatctgtccatccaaccctggattgccttttggtaatctttggcgggagagcgaccgctcacgtgcgtaataaagaagaaattgaatggcatatgggctcctggtttcccctcaaactgataatagtccattgttctatcgagagttgtatatgcttctgtcattagtacttttgttttcccctctttctttgtgtagaaatcgaagacctccctgaaccttgttatcaatctatatgtattcggttggtccatcgtccgcgaccggttgtaattccagtagttcgtcgggtcgagatcgggcgagagcaattcttggtctcgccattttacgtgccgttacaccggttaccgacatcaggttccttcaaatcggacacgggcgcccctccccgaggggatgccaatggggggaggtcccaggccgaagcctgactttctactacctccggagctgtgcccttctctgcacgtcccagttgagcactggtgggctgacctcggggacaaggtcgccttaacttaccg

SEQ ID NO: 12 - RNA, 939 nt, synthetic construct
  source 1..939: mol_type = other RNA
agcagcttcttgcctctgattccacggtttcttcttaaagggccccgacgactgctgcgggccttgataaggcgcgctcctgttatttgcctcacggaacgtcttttccgcggccatcattgcgtccattgatcggatcaaatcttgcctcattgcatccacggctcgagtattcctatccgtatccgcacgatttagatcaactgcgtgtaccaaagtcgctagggcgttctcattggccttcacccgggattctaaggatgattcctgccccgtatagtgatttacggccaaaatagcgccccttcctttgctggtcgcggctactgctagcttcgcattttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttccgtcgagttaactttagccaagcccgctagttttctcttcgcttgaacgtaatccaacgggtcctcattttctccctgcgttcgcgccgagaatttcgtgagggcatcctcgtcgctgtcaaagtattggatcaatttcttctttacttcctcaaaagtcctgcagttaccgaacgctacctcttcattgtcgtagtactggatggcacgtttcgctaagtgatttctgagttggtcccgcttttcttgatccgaacatttcttatagaaattttcaaaatcttttagaaattctctaacgtcgtagtcagcttctcctttgaatagttttctaaacggcggtgccttaatcgtcaccgtaggt

SEQ ID NO: 13 - RNA, 668 nt, synthetic construct
  source 1..668: mol_type = other RNA
ttgcgttcctgctccctttgccctttaccgcgctcaattatctctattagaaccggagatattcggtttacaaaaattttttggggcccagccccccttaatcctttccctatggacttcctatatggccccagaggtagcccccgggggttaggcaaataatcccaaaaaattcccaaattctaacggaaatgtggcactaccgcccctacgtcactctggctatgacgtagttgatttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgattttacgtgccgttacaccggttaccgacatcaggttccttcaaatcggacacgggcgcccctccccgaggggatgccaatggggggaggtcccaggccgaagcctgactttctactacctccggagctgtgcccttctctgcacgtcccagttgagcactggtgggctgacctcggggacaaggtcgccttaacttaccg

SEQ ID NO: 14 - RNA, 1848 nt, synthetic construct
  source 1..1848: mol_type = other RNA
atgtcaacagcattaggaaaaatggtggatgaagatagagaaggtagatttgggtttgtgtatgcagtatcaggtcctgtggtaacagcagagaaaatgtcagggtctgctatgtacgagctggttcgagtaggttactttgaactggtgggtgaaatcatccggctggagggtgatatggccaccattcaagtgtatgaagaaacatcgggtgtaactgttggtgaccctgtgttgaggacaggcaaacccttatctgtagagcttggtcctggtatcctgggtagtatttttgatggtatccaacgtccactgaaggacatttgtgagttgtctcagagcatttacatcccaaaaggagttaacattcctgccttgaacagagatgttagctgggagttcaatccaatgaacttaaagattggtagtcacatgaccggtggagatcagtatggtattgtacatgagaatacacttgttaaacataaaatgatcatgccacccaaagcaaagggaactgtaacatacattgctccagctggtaattacaaggtagatgaagttgttattgaaactgaatttgatggagagaagagtaaatacactatggttcaagtatggcctgtacgtcaacctcgccctgtcaccgaaaaactccctgcaaattacccccttctaacaggtcaacgagtccttgattctctgtttccttgtgttcttggaggaacaactgccattcctggtgcctttggctgtggtaaaactgtcatctcacaagctttatctaagtattccaattctgatgtgattgtctatgtaggatgtggtgaacgaggtaatgaaatggcagaagtattgagagatttccctgaactttcaattgaggtggatggagtcacagaatccatcatgaaacgtaccaccttggtagccaacacatcaaacatgcctgtagctgctcgagaagcttctatttacactggtatcacactgtctgagtacttcagggacatgggttacaatgtgtctatgatggctgactccacgtcacgatgggctgaggctttgagagaaatctcaggacgtctagctgagatgcctgctgacagtgggtatcctgcttacctgggtgcaagacttgcctccttctatgagcgtgctggtagagttaagtgtttgggtaacccagacagagagggttctgttagtattgtgggtgctgtgtctccccctggtggtgatttctcagaccctgtcacttcagcaacattgggtattgttcaagtgttctggggtcttgacaaaaaacttgctcagaggaaacattttccatccatcaactggctcatttcctacagtaagtacatgagagccctggatgatttctatgaaaaaaaccaccctgagtttgtacccctcagaacaaaagtaaaagaaattcttcaagaagaagaagatttgtcagaaattgtgcaactggttggtaaggcatccctggctgaatctgataagatcactttggaagttgccaaactgctgaaagatgatttcctccaacaaaacagttactccccctatgacaggttctgtcccttctacaaaactgttggtatgttgcgtaatactattgctttctatgatatgtcccgtcatgcagttgagtctactgcccaatcagaaaacaagatcacatggtctgtgataagggacagtatgaacaacatcttgtatcaactttcgtccatgaaattcaaagacccagtcaaggatggtgaagctaaaataagagcagactttgatcaactctatgaagacattcagcaagcattccgtaacttagaagactaa

SEQ ID NO: 15 - RNA, 1584 nt, synthetic construct
  source 1..1584: mol_type = other RNA
atgtcgatatctaccaagcaggctctacgggaaaacgtcctggctgtcacgcgggattacatttcgcagccgagaatcacatacaaaactgtgtctggtgtcaatggacccctggtcatcttggatgaagtgaagttccctaagtatgctgaaattgtacagctccgtttgaacgatggatcttaccgtgctggtcaagttctggaagtcagtggctctaaagctgtggtccaggtatttgaaggtacctctggaattgatgcgaagaacactgtctgtgagttcacgggggatatcttgagaacaccagtgtctgaagacatgttggggcgtgtgttcaacggaagtggaaagcctattgacaaaggaccccctatcctagctgaagactaccttgacattgaaggtcaacccatcaacccctactcaagaacctacccacaggaaatgattcagactggtatctccgctattgatgtgatgaactccattgcccgaggacagaagattcccatcttctccgctgctggtctaccccacaatgaaatcgccgcccagatctgtcgacaagctggcctggtcaagatgccgggcaagtcagttctggatgactcggaagataacttcgccattgtgtttgctgccatgggagtcaacatggagactgcccggttcttcaaacaagacttcgaggaaaacgggtccatggagaacgtgtgtctcttcttgaacttggccaacgacccgaccatagagcggatcatcactccccgcctcgccctgaccaccgccgagttcctcgcctaccagtgcgagaagcatgtgctcgtcattctcaccgacatgtcctcctacgccgaggctctgcgtgaggtgtcagctgcccgggaggaagtccccgggagacgtgggttccccggttacatgtacaccaacttggctactatctacgagagggccggacgtgtggagggcaggaacgggtccatcactcagattcccatcctgaccatgcccaacgacgacatcacccatcccatccctgacttgaccggctacatcaccgagggccagatctacgtggacagacagctccacaacagacagatctacccccctatcaatgtgctgccctccctgtcccgtctcatgaagtctgccatcggcgaggacatgacacgcaaggatcactccgacgtttccaaccagttgtacgcctgctacgccataggcaaggacgtgcaagccatgaaggcggtggtgggagaggaagctctgacacccgacgatctgctgtatctggagttcctcaccaagttcgagaagaactttgtgagccaagggaactacgagaaccgcaccgtgtacgagagtctggacatcggctggcagctcctgcgtatcttccccaaggagatgctgaagagaatccctgccgccacgctggccgagttctacccgcgtgactccagacacactggcgccaagttaacacgcgcgctggtcacccctccacccccctaccattgtctagacaagtgggaaccttacagcccctctaccccccctaccattgtggtctag

SEQ ID NO: 16 - RNA, 732 nt, synthetic construct
  source 1..732: mol_type = other RNA
atgtcaggtaaagatagacttgccatttttccgtcccgaggagctcagtccctcatgaagtcccgtctcaagggggctcagaagggacacagtttgcttaagaagaaagctgatgctctccagatgagattccgtatgatcctgagcaaaatcatcgagacaaaaaccctcatgggtgaagtcatgaaagaagctgccttctccttagcagaggcgaaattcacaacaggggatttcaatcaggtagtcctacaaaatgtaaccaaggcacaaatcaaaatacgcactaagaaagacaatgttgcgggtgttactcttccagtgtttgagagttaccaggatggtacggatacctacgagctagctggtcttgccagaggaggtcaacagctcgcaaagctgaagaaaaactatcagacagccatcaaactccttgttgagctagcctctctacaaacatcctttgtaaccctagatgatgttattaaaattaccaaccgcagagtgaacgctattgagcatgtcatcattcctcgtattgaaagaactctggcgtatattatttctgagctggatgaactagagagagaagagttttaccgtttgaagaagatccaggataagaagaaggtgatcaaggcagcatcagaagcattcagaaagtcccgtaaatatgatgaggagcaagcaatgaatatgcttgatgaaggtgatgaagacattctgttctaa

SEQ ID NO: 17 - RNA, 681 nt, synthetic construct
  source 1..681: mol_type = other RNA
ttaatcatcaaatttacggttagggttacgaccaaatatagcaactctaatgtcagggatgatctgttgtgcaatcaattccagacgagcctccaaggtattgctgatcttgatttttcctctttgagccaagagctcaatacctcctgtggtattaacaggtaagaattgttcactgtcaagtttcaagttaacttccttaccagcaacatcttggtattgcttggccaccgtaggtaagacaccattgacaagttctttgtcagcttcccgggcacggatcagaacattgggctccaataattgcagcaggccttgaaccattaacttttcaatgagttgggtgtacttggggcggtccttggacacctcacccagtctgttcctggcctcttccaacacatttctgacatgatcttccctcaccttcaaagccttcagtcgggcctgattcagcatgttagaggactgaatcttcttctgcagctcaacttgcttttctttcctgtcatagtattccataatcttgagacgctggtgttggaccaaacgccctttttcgatgttgaattcttcttctgccttagcatcgatttcctcagccttctcgttggcttcttgctcgatgaaagccatcatgtgattgatttgcttttgaacgtccgcatcgcttaaagccat

SEQ ID NO: 18 - RNA, 1131 nt, synthetic construct
  source 1..1131: mol_type = other RNA
atgtgtgacgacgatgtagccgctttggtcgtggacaatggttccggtatgtgcaaggccggattcgccggtgatgacgcccccagagccgtgttcccctcaatcgtcggtagaccccgtcatcagggtgtcatggtgggtatgggtcaaaaagactcctacgtcggtgatgaggctcagtccaagagaggtatcctcaccctgaaataccccatcgagcacggtatcatcaccaactgggacgacatggagaagatctggcatcacaccttctacaacgagctgagagtcgcccccgaggagcaccccatcctgctgacggaggcacccctcaaccccaaagccaacagagagaagatgacccagatcatgtttgagacgttcaacacccccgccatgtacgtcgccatccaggctgtgctctccctgtacgcctccggtcgtaccaccggtatcgtgctcgactctggagatggtgtctcccacaccgtccccatctatgaaggttacgccctcccccacgccatcctccgtctggatctggctggtcgtgacttgaccgactacctgatgaagatcctcaccgagagaggttactccttcaccaccaccgctgagcgggaaatcgtccgtgacatcaaggagaagctctgctacgtcgccctggacttcgagcaggagatggccaccgccgccgcctccacctccctggagaagagctacgagctgcccgacggacaagtcatcaccatcggaaacgagagattccgttgtcccgaggctctgttccagccttccttcctgggtatggagtcttgcggtatccacgagaccgtgtacaactccatcatgaagtgcgatgtcgacatcagaaaggacctgtacgccaacactgtcctgtccggtggtaccaccatgtaccccggtatcgccgacagaatgcagaaggaaatcactgccctggctccttccaccatcaagatcaagatcatcgctccccccgagagaaagtactccgtatggatcggtggttccatcctggcctctctgtccaccttccagcagatgtggatctccaaacaggagtacgacgagtccggtcccggaatcgtccaccgcaaatgtttctaa

SEQ ID NO: 19 - RNA, 1268 nt, synthetic construct
  source 1..1268: mol_type = other RNA
ctcaactcgggttatagtgcgctgtaatttaatgaaactccaacttgagggatggtcttgcttatcttaactttaatcctatttcataaaaaaagttctcggtccgaggaggctatacaactggtctacacaatcaccttatgaagctctttgttgttgtaaccagcgtggctacagctgggggagtggtttgctttcactttgcttccgaaatacttggtctggaccaaagttattactgcagtggccgatgtgctatcttgaattcagggcagagttgttctgaacatggcagtgacttcatcatccgagagcttcgagaacaatcattctctcctgtggaacgttctccccgtgattgtcttcttctccgtgttcttcacttcaggccgagctctgttcaacaccttgaggaagtccgaggtcttcgcccgcatgcgggagtgaatgtaggccttggaacatttgatgtggtagtgcagatagtcccggaacatgtggatcagattgatggtgttctctcgggccacccggtttgtgtgtctgggaaacagcacaaacgtaatgtagccaatgttgtccccttgtcgggcgtccgtgtctctcagctccaggggcggctccttgtgactgaacagcacctggggcgccgtgtgactcgcccgccgcccctccttgaactcctgcatgaacaccttgcctatgatgacgtcatcctcaccccggaatactgtgctgaacaccaccgtcacgcggtctgccttcgcctccacatacatagtttcttcattcctatagttgatcacggctctactctgaccttcctctcctctttcttgaaagtcaaagtatttttcaaacacagatgcaaaacaattcctctttaagagaccaatttttttgactagttgctcccagtcagaaggaatattttctaggtctatcagtacagatgcattgtagcctggttcaggatcagtcaacagagcaccatactctcgcctcaacagttcatcagcaccatgttgctcaagctctttgtagaacttgagagatatactagtccttattttagttttatcaccactaaggttggaaatgtgaaataatactccatcaaaatctgctattgtgatatctactgattctggttttgatccagcgatggcatttttgaatttcaaagaaagagtttcctccacgatcctgttgtgtatttccaagagaatcatgttgaaaattaaaatattactacaattccataaaat

SEQ ID NO: 20 - RNA, 816 nt, synthetic construct
  source 1..816: mol_type = other RNA
atgaaacctaacgcaacacctcctgaacagaaaacgtcaagtttgttgacacgagatgaaaatgaagctgtgttcaagcttttgggtaatagatgtcaggcgctttccacaactgtgatacagctgttcaccacagacggccctaacgataacgagtggcacaagaggtgtttcggcattctgtgccttgtcaaggataatccccgcaaatcctatttcttccgactctactgtttaacgaggagacaactggtttgggaacatgagctctataaaggcatgagctacatggccccacagaacttcctgcacacattcgaagccgaggattgtatcgtggccttcaactttgccaatgaggaagaggcccggcacatgagatacgtgattctggagaagcagaaacgattggagagaagacaccgggcctccactcagcctcgtcactcctccaccccgggcctggacagagagcgaagccgaaccctgcagccagccgccatgaccaacgggaccaaactgtcgcccgcggagagagcaagacatgtgcgatcctcgtccggcggaggaggcggaaaccggaagagggaggccaagcggcgaggcaacaagctgacgaaggcggacatatcctcccccacggggttcagacatgtgtcccacgtggggtttgaccccaacaaggggttcgacgctgtggacattcagaacagccccgagctggagatgttctttgagaaggcgggcgtctcgcagagtcagctgcaggaccgcaagacccgcgagttcatctacgacttcatctcccgcaacggc

SEQ ID NO: 21 - RNA, 1566 nt, synthetic construct
  source 1..1566: mol_type = other RNA
atgtggaaatcagcagcaggttctgcaatagcaccagttgttccggacgaagatgatgactgggaaactgatcctgacttcatcaatgatgtaagtgaacaggagcagaggtggggctccaaaactataccaggttctggtagagatgctgggtctattgacatgaagcaactgcgggaagaagtggctatgtcagatgcatgctacaagcaaaagcagttagatggaggatcaaaagcttcttttggatatggaggaaaatttggtgttgagaaggataggatggatcagtcagctgtaggacatgactatgtcgcacagcttcatcaacacgagtctcagagtgattacaaaactgggtttggggggaagtttggtgtgcaaaatgatagagttgataaaagtgctctgacttgggagcacaaagaagtaatagaaaaacacacttctcaaaaagattatagttcaggatttggaggtaagtttggagtgcaaaaagatcgacaagataaatcagcagtaggatgggaccatcaagaaaagattgagaaacatgaatcacaaaaagattacgctaaaggatttggtggtaagtttggcatagaaagtgatagacaagataaatcagcagttggatgggaccatgtagagaaagtaaacaaacatcagagtcaaacagatgctaacaagggtgttgtaagttcttcaaaagtcaaagagctgatagctgccaattccaatacttccattaaagaaaatgtcaaaccaaaacctgacatcagtcatgtgaaaccatccaacctaagagcaaaatttgaaaacttggcaaaacaaacagaagaagaaagtagaaaaagaagtgaagaagaaaaagaaaaaagaaaactaaaggatcaaatagatctccaacaggctcaaaagttagaggaaagacgtctatcagaattgcaagtaaaagaagctgaagttgagagaaaaatgaatgcgcactcaaatgtgccttcttcaccaacaagtgattcaattccagtcaagtcaatactaaaacaatcatcaattgagaaaataactgttcaaaatagtaatgatgaagagaaagagaaacaaaaaatgattcaagaagaaattgagaagaaaaatgagcttgaaaaagaaagaataaagcaagaacaagaaattaaaaaaagaaaagataaagaagaaaaagatagacaagaaagagagcaacaagaaaaagaacaaaaggaaagagatgaaaatcaaaaattactccttaaaaaacagcaagaagaagatagactaaaagctgaggagcaagcaagactcttggaacaagagaggctaatggaagaattaagacttcaaggaaatgatgataacacagaggaggatttgggctatacagcaatagcactttatgattaccaagcatctgctgatgatgaaatttcttttgaccctgatgatattatcactaacattgaaatgatagatgaaggctggtggaggggcttgtgccatggacagtatgggttatttccagctaattatgtttccctccaacaa

SEQ ID NO: 22 - RNA, 1506 nt, synthetic construct
  source 1..1506: mol_type = other RNA
atgctttctgaggccaccctcgagcacttgctcaagaactaccgctgggtcctcgtgatattcgtcctactgcctctctccctcctctacgacatctaccacagcgtaggacagctcatcacggagtactgcagggacaagagccaggaccatggcaagaaagtgaaccatgttcaaagtcaggttcgagcctggctggcaggggggcagacctcccccatgtgcaccgccagggctgggtggaagagcatgaccctgcgggagcccaagtacaaggcgaccatgttccctgtggacctgggacctctggattccatcctctctgttgacgaacacagtcatacggttctcgttgagccttacgtgaccatgggtcagctgacacgctacctcattcccaagggctggacaattcctgtggttattgagttggatgacgtcactgtaggcggcattgtgtctggacaaggcctggagtcgagctctcacaagcatggtcagtttcagaatacatgcgtctcctatgagttggtcctcagtgatgccagtgtggtccagtgtagcaaggaaaatgaccccgatcttttctacgctgtgccttggtcttatggaactctgggatttctgacggccgtcgagatacaacttattcccgttaaaaaatacgttcagctccagtatgtggctctcaagtccctgccggatctggaacatcacctgaagaaagaggcagaaaacaaaggcaacgactttgtggaagccattgtgttctccaaagaccagtctgtccttatgataggcaccttctgtgataccccggaaccaagcaaaattaaccgcctgggtcgctggtacaagccttggttctaccagcatgtgaggagttaccttagcaggaagaagtacgcggaggagtacatccctatccttgactactaccatcgcttcagcacatccttgttctgggaaatacaggacattgtccccttcgggaaccaccctctcttccgctacgccctcggctggctgatgccccccaaagtgtccctcctcaagctgacccagacccaaaccatcaagcagctgtacgacaagcatcacgtggtggaggactatctcctgcccctgggagaggtgagggcgtttctgcagcatatccatgaccaaatacaggtctaccctctctggatctgtcctttccttctcaaggatcttcccggccttgtacatccttccaagcctggggattgtctctatgtggacgtgggaatatatggagaaccaaaggcgcaggattatgacagcaagaaaaccattctggatgttgagaattatcttggcaaaattagaggatttcagatgctctacgctggttgctatgagtccagatctgagttccggcacaactatgaccacagcctttacgatagcgtcaggtccaggctggcctgtgagaaagcgtttcccgtcatatatgataaggtgaaccgtggtgtccgagattga

SEQ ID NO: 23 - RNA, 1185 nt, synthetic construct
  source 1..1185: mol_type = other RNA
ttgcaggacctgagtgaggtgggctacggacacctggactctgccaagaagcgtggcgcccacaaccactacacgcgcggctacgggaaatccaaggcgcacatcatcaacgaaatgcgacgggcaaggaaaatcggcaacctaccgcccgtctacccgaacggttggttcgcgttgatggaatcaagcgagctgcgacctcgggacgcgaaatacgtttcggcgctcggggagaatttcgccgtgttccgatcggaatcgggcgaggtgcatgtgctggatgcctactgcccgcatttgggcgccaacatggcgatcggtgggttcgtgcggggggactgcatcgagtgtcccttccatcagtggcagttcagcggacgcgacggccgctgtgtgaacattccttacagcggaaaagtgccggaggtggctcgtgtgaggcactggcagtcagtggaagtgaatgactttgtgttcgtgtggtaccacgcagaggaggaggacccatcctggcagccggaaccacttgacaaaatcacaagaggggactggcgctaccggggcagatcagagtacctcatcaactctcacatccaggagatcccagagaacggcggggacatcgcccatctgaacgccatccacgccccttccctcgtggcgggcagcaacctgcacgacctggagaccaccgccgcccagtccgcccgccacgtgtggcaggccacctgggagccgcatacggcggccggagaaacacacgtggccaccatgcggctgcggcatgacctgcggctactcgaccgcatcccgctgccgctcattggcatgaacgtcgaggcccgacaggtgggcccgggctacgtggaaatgatcatgaccacaagcataggtcgcctcgccatcctccagacagtgactccggtggaacctatgctgcagagagtgattcacaggatctacgccccgccccatctcttctggtacgccaacatcgttctctacggggagtgcatcatggtgagtcgagacatcatggtgtggaaccacaagacctacatagacaagcccctcctggtgaaggaagacaagaccctggcccgacacaggaggtggtacagtcagttctacaccgagaacagtccccgctacgagtccaagaaggatactctggattggtgatag

SEQ ID NO: 24 - RNA, 849 nt, synthetic construct
  source 1..849: mol_type = other RNA
aattaatttaattcacttcaatttaattaattatcattgttctagtacaataggtttaccaaaaacacttccaccatgcaagcaatcaagtgcgttgtggtaggagatggtgccgtgggtaagacctgtctcctcatcagctacaccaccaacgccttccccggcgagtacatccccaccgtattcgacaactattcggccaatgtgatggtggacgggaagcccatcaacctcggcctctgggatacggccggccaagaggactacgaccgacttcggcccctctcctacccgcagactgacgtgttccaaatctgtttctcgctcgtgaacccggcctcgttcgagaacgtgcgagccaagtggtaccccgaggtgcggcaccactgccccaacacgcccatcattctggtgggcaccaaactggacctgcgggacgacaaggagaccattgagaagctcaaggagaagaaactggcgcccatcacatacccccagggcctgtccatggcgaaggagatcggggccgtcaagtatctggaatgttcggccctcactcagaagggcctgaaaactgtgtttgacgaggccatccgcgcagtgctgtgtcccgtacctactgttcccaagaagaaacggtgcgcgatcctgtaatcgaacccgcggtcaccagcatacagagatgaagcccccgcctaccaccgcagcccttgtgtaaaatatcatgtctttgactcgccagtatttgcaggcgcggccggtgggaaaatgttttgggggtgctgccacatgaaccgaactaaaatgggatttatagagtcgctcgctcagtaatgggtttgtttacca

SEQ ID NO: 25 - RNA, 1377 nt, synthetic construct
  source 1..1377: mol_type = other RNA
atgatacagaagtctccatctatacaaatcaggatgacttcactaggattatggagtctggaaggtaccccaggtatcttggaagtctataatcatgtagactccccaccagaagaccaaggagatgacacgagcagccacacgagagcggaggttgcctccaaactattggaaacacttgacaatttcaactatgatgaaacagaagagtcgtcggacgcctacatctccgactggctcaccggctcggaggcggctgtggctctgccctccatcttcgaagacctcgcctccctgccccccccgcctcccctgcccctagtgcccacgggccgccaagcagtctgctcctcaggcccgggcgggttctacgggccccgcggcgcatgcgcagtagtccccactgtccccgtgatcgacaagctcgggtacggagcgagtgcggcgctagtatggaactcgaataagaaggttggctacgaagcgaccgccttgtggacgccggcgggctccgatgactattccaccggctccctctactctccgcccgccgcgggcacccagtggcacctggagccggtgtaccagggcgtgtccaaccagctgacgccgccgcacagtccgcccaccatgtacgagaccagcccccgccgcccgctcgagtttgatgatctgccagcagacttgctgaaatcatctgccgccaaggagctctacgaagacctgcctgccgagtccgccctcaacagcaaggaagaaaacaacaacggccagctgatcatgtccctgctggcggagatggaccagaaggacattgacgagatcgtgcagtgctccgtggaacagaacttcggaatcgcgcctgattccgaacccaacttggaagcatccgccgcgaccaactacgtcgaatcgctgagtcccgaacactcgtgctcttcatccgaatccaacttcggctatcattccgagtccgaccgaagctcgtcgtgcagctccgaccccgactacaatccctactcgccgcccgaagtgacgcgaaaaacgcccgaaacaagccgaagtggcggtggcggacgaaaagcggcgaaaacgagcgaagccaaccgaagtgctcgagcgatcaaaccgtacgcccgcaaagcggccgtgcccgtggaagacaagaaactgcgcaagaaagagcagaacaagaacgcggccacgcgctatcgaatcaagaagaaggcggagatcgaagtcattctgggcgaggagtcggaactcagggagaagaacgaggagctgcagaagagtgtggaggatttgaatcgcgagatcaagttcatgaagaagttcatgcgcgactttttcaaaacgcaaggtgtgctcaagtga

SEQ ID NO: 26 - RNA, 2961 nt, synthetic construct
  source 1..2961: mol_type = other RNA
atgatattgtggaaagatggaagagcattcagtgaaatattattcctattgttagccattatattcctagcttcctccacatacagtacacagactgtgaggaggcggctgcggcggccctccatcaagtccaagccgtccgaagagtccggggaccaggatgtgtccgccagcatcaacaggttcaagctcaagagccgcacctccgcctccagcagcgccgtggaggcggccgccagcggcaagtccagtgtgaaggcgggcaaggatgaccccaagtacaaggtcgtctgctactacaccaactggtcccagtaccgcaccaagatcggcaagttccagcccgaggacatagaggctgacctctgcacgcacatcatcttcgctttcggctggctcaagaagggcaagctgtccagcttcgagtccaatgacgagaccaaggacggcaaggtggggctgtatgagaagatcgaccagttgaagaaggccaaccctaagctgaagacgctgctcgccataggaggatggtcgttcggtacccagaagttcaaagagatgtccaaatccagatactcccgtcagaccttcatctactccgctgtccccttcttgaggcagaggaatttcgacggcttggacattgattgggaataccccgccggaactgaagacaagaagaactacgtactcctcctgaaagaattgagagaagccttcgaagcggaagctcaggaagtgaagaagccccgcctcctgctgacagctgctgtccctgtgggccccgacaacatcaagggaggatacgatgtccccgccgtggccggctacttggacttcatcaacctgatggcgtacgatttccacggcaagtgggagcgggaggtgggccacaacgcgcccctgcgtgcgcccagcaccgacagcgagtggcggaaacagttgagtgtcgagaacgccgccaacatgtgggtgcgtctcggcgcccccaaggagaagctgatcattggaatgcccacctatgggcgcaccttcaccctggctaaccccgccgtgttcaaggtgaacagtcccgccactgggggaggcaaggcaggagagtacaccaaggagagtggtttcctcgcttactatgagatttgtgaaatgctgaagaacggcgcaggctacgtctgggatgatgaaatgaaggtcccctatctggtgcatggtgaccaatgggtgggctttgatgacgagcgatccatccggtacaagatgaactggctgaaggacggggggtatggaggtgccatggtgtggacagtggacatggatgacttcactggaacagtgtgtggaggagacgtcaagtacccgctcattggagcaatgagagaggagctgaacgggatcccccgagctaacaatgccaaggatgtggactgggccaaggtagccggcaccctggacgtagaatccgccactaaacccgccgccatcaagatctccgtggcggatgtgctcagtaaggccaagaagatcaccatccagaaggtcgacaccagcaacttgatcgattccaaagttcgccccgcccagatcttctgctacatgaccagctggtcccagaaccgacccggagctggcaaattcgccccagagaacattgaccccaaactctgcacccacgtgatctacgccttcggaaccttgaaggatcacaagctgaccttgaacgaggagaaggagaaggatgaggcaggcaaattcgaggccctgatgcaactgagagagaagaaccctgatatcaagatcctgctagctatcggaggctgggccttcggctccacccccttcaaggagctgacaggcaacaccttccgtatgaaccagttcgtgtacgagtccatagagttcctgagggagcacaagttcaatggcctggatgtggactgggagtaccccagaggtgccgacgaccgagcttcctacgtgaacctgctcaaggagcttcgtcaagctttcgaaggcgaggccaagacctccggtgagccccggctccttctcacagccgcggtgccggctagcttcgaagccatcgcagccggctacgacgtgcccgagatctccaagtacctggacttcatcaacgtgatgacctacgacttccatggccagtgggagcgacaggtcggacacaacagcccgctccaacctctggaaggtgccaccagctaccagaagaagctgacagtggagtacagcgccaaggagtgggtgaagcagggcgcccccaaggagaagctcatgatcggtatgcccacctatgggcgcagcttcactctggtggacccgagcaagttcgacatcggggcgccagcctcagggggcggcgaccctggcaagtacacagccgaggctgggttcatgtcttactacgaggtatgcgacttcctgaagaaagacaacactaccctggtgtgggacaatgagcagcaggttccattcgcctacaaggatgatcaatgggtcggatttgatgatgagcgcagtctgaaaatgaaaatggactggctgaaggaagacggctacggaggcatcatgatctggagtgtggacatggacgatttcagaggccagtgtggctccggcaagtaccctctgatctccaccatgaagagcgagctagatgactactccgtggctctggagtacgacggaccctatgagacctacagccccaccggcaaatacacgaccaaagaacccaatgccgtgtcatgcgaggaagaggacggtcacatctcctatcacccggacaaggccgactgcacccactactacatgtgtgagggcgagcggaagcaccacatgccctgcccctccaacctggtgttcaaccccaaggagaacgtctgcgattggcccgagaacgtagagacctgtttacaccacacccaagccccgcctccatccaaa

SEQ ID NO: 27 - RNA, 4728 nt, synthetic construct
  source 1..4728: mol_type = other RNA
atgagcaccctgcgacagctacacctgcaggactcggaccccgaggagttctccgaagacgaaagctcccccttgtctcccaacgacatctatggcggcagcacccgcacagtgcccgagacgaaggcttgggatgtgttccgggacctgcccccccgacaagactcggggtccatggcctccaaggcctgcctggagttcacaatcaaggccctcaagatcttcgtctacctcttcacctttgtggtggtcctgctctccggggttgtggccaagacagctgtgttcttcatgacgtcacaactccgcgcccccgacaagcacattccctactgtaacaaggagtttggtcgagacaaacaattcgcggtgtacctgccttcagaggagcggatcgcctggtactggtgtctcatcatagcctttatagtccccgagttgggaaccgctgtccggtctatcaggatctgctacttcaagtcgtggaagaagcccccactgcacgactttatcttcgtcatcaccatggagctgctgcatacgctgggtctggcctgcttgatgttcgtcctactacccaacatggatgtggtgaaaggcgccatgctcactaactgtctctgcttcctgccagctttcctcacgctgatatctcgcaacaccaggggcctaaccaaggggtcagagaggtccgaggtctatctgaaagccatgatggatatgatagccgtgtcagcccaggcgacaggggcgctgctgtggcctctcctggaggggcacaatgacccttgcctctggttgattccaccggccctgttctgtgtgtcctgtggctattgggagaactatacaagcaaacattccattttcgggttcatgaagagcgcctggcgagtgaaggagagactgaagcgcacgcgctacttcacctacgcctttgtgtccttcctcaagatcctagtcttcttctgctcggcgctggtgttcatgtccttccgcggggagggggtgggggagttcttctccaagtttcacgcctccttcgcccaacacaagatcaggatctacgaggtgaaagcctcagcgtttggaacttctatcccggacctggctgatccaaacctcacgggagactaccgggatattgattctgaggataattttgccctcaaggtcctgcttttccagatattcgctgcctacctatgctacattgtgggcaagtacgcgtgtaagatcatgatccaggggttcagctacgcgttccccgtgaacctcaccatccccgtcaccatctctatcctcatcaccatgtgcggactccgcaatgacgacccctgctttttccagaactccattcccgactacctctttttcgtgtctccctccatttacttcctcgacgactttattatcaaacagcatgcatgggtgtggttgttatggttactgagtcaaacttggattacgctacacatctggacaccaaaatgtgagaggttggccaccacggagaaattattcgtgagacccatgtacgattctttgcttatcgatcagtctatgagtatgaaccgtagatgtgatgatgagaaggacgtcaaaactgaggaactcgtggatcgcgagaaagaccctgacgaatactacgagacaatctccgttcacactgacgcttcctccactacacccaagactgtcaagaaatccgactccatcacgcgcatctacgcatgcgccacaatgtggcacgagaccaaggacgagatgatggagctggtgaagtctatcctgcggctggatgaggaccaatgcgcccgccgagttgcgcagaaatatctgcgggttgttgatccggactattacgaatatgaaactcacatttttttcgacgatgctttcgaaatctccgacgtgaatgacgagtgggcccaggtgaatcgcttcgtgaagttgttcgtgtcggtgatagatgacgcagccgcccatgtacacgagaccaacatacgcatccgaccccccaccaagtaccccaccccgtacggagggcagctcatatggaccctgccggggaagaccaagatgatcgtacacttgaaggacaaggcgaagattcgacacaggaagcggtggagtcaggtgatgtacatgtactatctgctgggccatcgtctgatggagctgcctctgcctgtggaccgcaaggaagtgatggccgagaacacctacctgctcactctggacggcgatgtggacttccaacccgacgctgtgaggctcttggtggacttgatgaagaagaatcgaaacctgggcgctgcctgcgggagaatccatcctgttggtggaggacctatggcttggtatcagaagttcgagtacgccatcggtcattggcttcagaaagccaccgagcacatgataggctgcgttctgtgtagtccgggctgcttctctctcttcagagccaaggctctcatggatgacaatgtgatgagaagatacaccaccaagtctgcagaggcccggcattatgtgcagtacgaccaaggtgaggaccgatggttgtgcacgctactgctgcaacgaggctaccgtgtggagtactcagcggccagcgatgcctacacccggtgccccgagagcttcaatgagttctacaatcagcggagacggtgggccccttccaccatggctaatatcatggacctgctcatggactataagaagaccataaagataaacgataacatctcgatgccctatatttggtaccaggtgatgttgatgggaggtaccatcctgggccccggcactatattcctcatgttggtgggggcctgcgtggcagtgttccacattgacaactggaccagcttctactacaacatcactcctattctcttcttcatgtttgtttgcttctgctgcaagtccaacatacagctcctggtggctcagatactctccacagcatacgccctcatcatgatggctgtgatagtgggaacagctctacaactgggggaggacggtattggctctccgtctgccattttcctcatagtcatgttggggtccttcatcatagcggcattgcttcatccgcaagagttcttctgcatagtcttcttctccatttatctcttatctgtgccgtccatgtaccttcttctcattctctactcactcatcaacttgaacgttgtctcttggggtactcgagaggtcgcagtgaagaaaaccaagaaggaactagaggaggagaagaagcaagccttgcagtccaagaagaaagccaagaaccctctcctgggcttcttgcaaaactacacgcccgacgctggggaggatgaggagggcagtctggagctttcctttgctggactcttccgctgcatgttctgcacctaccccaagccggtggacgagaagcagcaacttctccgtatagcggacagcttgagtggactggagaagcgactggatcacattgagaggatcgtggaccccaacatgcccaaccctcatcaccacaggcgccgcactacatcagccagcagcagcgcgcaccatgcaccactaggctccgtgccagagaacaagaccgaagatcagtccacggacgaggaggatgaagacagtgatactgtgtccacagcagagcccaagattgagcgtaacgatgacattaacccttactggatcgaggataaggaattaaagaagggtcctgtagcattcatctcacctaatgagagacaattttgggtagaactcattgataaatatctctaccccatcgataacgacaaggcggagcagagccgtatagcaagtgacttgaaagatctacgcgaccaaagcgtgttcagtttctttatggtgaacgctctctttgttgtgatcgtgtttttgctgcaactcaataaagataagttgcacgttcgctggccctttggaatcaaaacgaatattacgttcaatgaggcgacacaggaggtgatcattactaaagagtacctacagctagagccgattggtctagtgtttgtgttcttcttcgccctcattctggtcattcagttcactgccatgttgttccatcggttcggaaccatctcgcacattctcgcgtcaacggaactcaacttgtgttgcaacaaaaagactgaagacctgtctcaggacgggctcctagacaagcaagccgtggacatcgtccgtcacttgcagaggttgcggggcatcaacggagaatatgagaacgacagtggctcctcaggtgaccgggtggggaggaggagaaccatctacaacctggagaagcagaagaacaagacaaggacgataggaactctggacgtggcgttccggaagagattcctccagatgaagatggaggatgtggtggagacagcaggtacaccagtgctggggcgcaagttgaccatgaggagagagatacgagaggctttggaagtgaggcgtcgctccttacaagcagagagacgaaagtccaacatgcaaaccctaggcgctcataacgatatttatggacgaactcagaggatctcaaacgcgggatctcacgtgaaggacttgtttgacgggcatcagaaccctgcgtacgagcctgacctcgcctcggacagtgaccgacccctgcccggggggcgcgctgtcaactgggcggagctggagagtcagaggatcaacagtcacatc

SEQ ID NO: 28 - RNA, 1472 nt, synthetic construct
  source 1..1472: mol_type = other RNA
gtggggggctggggggaggggggagagaagtattccgccatggtggcggacaagagcaagaggaccaccttcatcaggagcatcgtgggtttcatgaagaagtacaacctcgatggttttgatctggactgggagtaccctgctgctacagacagaggtggcagcttcagcgacaagactaacttcttctactttgtggaggagctgaggaccgcgttcaacagatttgggaagcactgggaaatcaccatggcagttcctatggccaagttcagactcaatgagggatacgatgtgcctgaactttgcgacctagtggacgcaattcatgtgatgtcgtatgatctgcgaggtaactgggcaggctttgccgacgtgcacagtcctctgtacaggcgaccttccgatcagtatgcttacgaaacgctcaatgtgaatgacggactccagctgtgggtggacaaggggtgctcccctcgcaagctcgtggtgggtattccgttctacgggcggtccttcactctgagcaacagcaacaacaactacaacataggcacctacatcaacaaggaggcggggggaggcgacccgggcccgtacacgaacgccaccggcttcatttcctactatgagatttgttcaatggtggagaaagatcatgattgggtacaaaaatgggatgacattggaaaatgcccttacacatacaaaggaaatcaatggattggctacgagaacccagagagtgttcaaatcaaaatggactggatcaaatccaaaggatacgctggagctatgaactgggcaattgatatggacgactttaatggactttgcggacccaaaaatgaactgctgagcattctgtacaggaacatgaaggactacaaagtgcccataccgcaggtctcgataacaccaaggcctgaatgggacaggcctcccagcacaacaccagaggacatagactacgcccctatttaccctccagtaaccacgagcaaacctgttcttgcatcgacttcccctactagaaaacccgtcctttcaaccgcctctcccaccgcgcccggagtttccagcacagaaaactctgctgtgatagaggcgaccactccccagaacaaaccccacaagaggccacgcccttccatcacggctaaaccccccagagcacatcaccgaggccggaagaaacctatccggagactcctgaggagaatcatcctgaaggcaacttgccagaacccagtggagtagattgctccagcgatggtttccaagctcatgctctctgcaataagtactaccaatgcgtcttcggaaaacccatagaattcacatgccgacctggtacgtacttcaatagaaaaatgagcgtctgtgattggccagagaaagtcgatgtcaataaatgcaaaatgatcaaaattctgaagacggtggactacgaagatgcaaaccaatttttg

SEQ ID NO: 29 - RNA, 423 nt, synthetic construct
  source 1..423: mol_type = other RNA
atgcctctgcccaagcgagtgatagaacctgtccacgtagcccgaggcaccatccctgatgagctgtccgctagtctaccttcggaacttgaggccgccaccaatggaaccctggctaacacggttcgtcaactgtccagcctcagtcgccacgcggaggatatgttcggcgagctcacgcgggaagcccacggcatggctgtgagggccaactgcttgcaggtgcggctggaccggctggctgtcaaggtcacgcagctagacagcaccatcgaagaagtgtccctgcaagacattcatctgaagaaagcgttcaagtcggccatcgtgtttgaccaggaggtggtgtcccgcagtactatnncaagaggtggtgtctcgcagcactatgcccacagccatgctggagacctacaagcaatg

SEQ ID NO: 30 - RNA, 495 nt, synthetic construct
  source 1..495: mol_type = other RNA
caaatcgtggaagtatggaacgaaatccagtcacaacagatgaacattctgaaagccttctacgttctcatgatcgtgcctctggagaccaacctggagaaagacaccaaggtggtgacggcggaacagaagaggttcctgcagcaacacaagatcctctccgagtcctacagcaaagccgcagccaacatgaagaagcaacgcaagaaacacaagagcaactccaaggcgagcaacacagtgagcaacaaagagatcaagtccatgcaaattctggaggaagagaagacaaagctggacgcattctgtgagcagggattgaaaaacgcgatgactcaggagcggcgccgctacggcttcgtgctggagcgtctgtccagcctcgccaagcactacctggcgtaccactctgccggggccaccctgtatgagaggaacatggaggcgtggggggaggtgaacaagacccgggaggcgctgcctgagagtgtggag

SEQ ID NO: 31 - RNA, 1848 nt, synthetic construct
  source 1..1848: mol_type = other RNA
atgtcaactgcattaggaaaaatggctgatgaagatagggaaggaaggtttgggtttgtgtatgcagtctcaggtcctgtggtaactgcagaaaaaatgtcaggatctgctatgtacgaactggtacgagtgggatactttgaactggtgggagaaatcatcagattagagggtgacatggctactattcaagtgtacgaagaaacatcgggtgtgactgtcggtgaccctgtgctaaggacaggcaaacccttatctgttgagcttggtcctggtatcctgggaagtatatttgatggtattcagcgtccattgaaagacatcaatgagttatctcagagcatttacatccccaagggtgtcaacattcctgcattgaacagagatgttagttgggagttcaacccaatgaatctgaagattggaagtcatatcactggtggagatcagtatggtcttgttcatgagaatacacttgtcaagcacaagatgatcatgccccctaaagctaagggtactgtcacttacattgcacctgctggtaactataaagttgatgaagttgttcttgaaactgaatttgatggagagaagagtaaatacaccatggttcaagtatggcctgtccgtcagccccgccctgtcacagaaaagctacctgctaactatccactattgacaggtcaaagagttcttgattccctcttcccctgtgttcttggaggaaccactgccattccaggtgcctttggttgtggtaaaactgtgatttcccaggctttgtccaaatattccaactcagatgttattgtgtatgtaggatgtggagagcgaggtaatgaaatggcagaggtactgagagatttccctgagcttaccattgaagttgatggagttaccgaatcaatcatgaagcgtactacacttgtagccaacacatctaacatgcctgtagctgcccgagaggcttctatctacactggtatcacactgtctgagtacttcagagacatgggttacaatgtgtccatgatggctgactctacatcccgttgggctgaggctttgagagaaatttcaggacgtcttgctgagatgcctgctgacagtggttatcctgcctacctaggagccagactggcctcattctatgaacgtgctggcagagtcaaatgcttgggtaacccagacagagaaggttctgtgagtattgtgggtgctgtatctccccctggtggagatttctccgaccctgtcacttctgctactcttggtattgtccaagtgttctggggtcttgacaagaaacttgcacagaggaaacatttcccctctatcaactggctcatctcttacagtaaatacatgagagccttggatgacttctatgataaaaatcatccggagtttgtacctctgagaaccaaggtaaaggaaatccttcaagaagaagaagatttatcagaaattgtgcaactggttggtaaagcctccttggcagaatctgataaaatcaccttggaagttgccaagttgctgaaagatgatttccttcagcaaaacagttactcaccctatgacaggttctgtcccttctacaaaactgtgggaatgctgcgtaacatgattgctttctatgatatgtcccgccatgctgttgagtctactgctcagtcagaaaacaaaatcacatggtctgtcatcagagacagcatgagcaacattctgtaccaactttcctccatgaaattcaaagaccctgtcaaggatggagaagctaaaaccagagcagactttgatcaactgtatgaagacattcagcaagcattccgtaacttagaagactaa

SEQ ID NO: 32 - RNA, 1490 nt, synthetic construct
  source 1..1490: mol_type = other RNA
tgtcgataagctcgaagcaagctttacgggagaatgtgctagctgtgacgcgggactacatatcgcagccacgaataacatacaaaactgtgtctggtgtcaacggacctctggtcatcttagacgaggtgaaattccccaagtacgctgaaattgtgcagctccgtctgaatgatggatcttaccgtgccggacaagtgctggaagtcagtggctccaaggctgtggtccaggtgtttgaaggtacatctggtattgatgctaagaacacagtttgtgagttcactggagacatcttgagaactcctgtgtcagaggatatgttaggacgagtgttcaacggcagtggaaaacccattgataaaggacctcccatcctagccgaggactacttggacattgaaggtcaacccatcaacccgtacagcagaacctacccgcaggaaatgatacagactggtatctcagctatcgatgtgatgaactctattgctcgtggacagaagattcccatcttctctgctgctggtctgccccacaatgaaattgctgctcagatttgtagacaagctggtctggtaaagatgccgggtaaatctgtacttgatgattctgaagataactttgctattgtgtttgccgctatgggagtcaacatggaaactgcccgattcttcaaacaagatttcgaagaaaacggttccatggagaacgtgtgtctgttcttgaacttggccaacgaccctaccattgaacgtatcatcacacctcgacttgccctcaccacagcagagtttttggcgtaccagtgtgagaaacacgtgttggttatccttacggatatgtcctcttatgctgaagctttgcgtgaggtgtcagctgcccgtgaagaagtaccagggcgacgtgggttccccggatacatgtacaccaacttggctaccatctatgagcgtgctgggagagtggagggcaggaacggatcgatcactcagatccctatccttactatgcctaacgatgatatcacccatcctatacccgatttgaccgggtacattaccgagggtcaaatctacgtcgaccgacagctgcacaaccgacagatctatcctccaatcaacgtgctcccctctctatcccgtctgatgaagtctgctattggtgaagacatgactcgcaaggaccattcagatgtgtccaatcagctgtacgcttgttacgctattggtaaggatgtgcaggccatgaaggctgtagtaggagaagaagctttgactccagatgacttgctgtacttggagttcctcaccaagttcgaaaagaacttcgtgtcacaaggtaactacgagaaccgcacggtgtacgagtccctggacatcggctggcagctgctccgtatcttccccaaggagatgctcaagcgtattcccgcctccacgctcgccgaattctatccccgggattcacgccacactggcgccaagtaa

SEQ ID NO: 33 - RNA, 732 nt, synthetic construct
  source 1..732: mol_type = other RNA
atgtcaggtaaagagagactacccatattcccttcccgaggagctcagtcactcatgaaatctcgtctgaagggagctcagaagggacacagtcttctcaagaagaaggctgatgctctacagatgaggttccgaatgattctcagcaagattattgagactaaaactctcatgggagaggtaatgaaagaagctgcgttctctctagcagaggccaagttcactactggagatttcaaccaggtagttctacaaaatgtcaccaaggcacagatcaaaatccgcactaagaaagacaatgtagcaggtgttactctacccgtgtttgagagctatcaagatggcacagacacttacgaactggctggtctagctaggggtggacagcagctggcaaagctgaagaaaaattaccagacagctataaaactcctcgtagaacttgcttcactacaaacatcatttgtaacattagatgatgtcatcaaaattactaatcggagagtaaatgccattgagcatgtcattattcctcgcattgaaaagacactagcatacattatttccgagctggatgagttggaaagagaagagttctaccgtttgaagaaaattcaagacaagaagaaggtgatcaaagcagcttctgaagcttttaggaagtctcgtaaatatgatgaagaacaggcattcaatatgctagaagaggaagatcaggacattttattctag

SEQ ID NO: 34 - RNA, 819 nt, synthetic construct
  source 1..819: mol_type = other RNA
atggtccaaaatgtattacagagcaacaaagttctgaaagattttagtcttaaactttgcgacaaatctacattccggtcgtacctgaaacttccgaacgcagccgacattttccgaccgcccccgacacttctcggcatggcgttagacgacgcagcggtggaaaagcaaatcgaacgcatggtggcgtttatccaaaccgaggcggacgaaaagttggacgacatccggcgaaaaatcgaagaggactaccagatcgaaagagagcgagtaacgcgagacggaaaggcgagcgtggatgaagaatatgctaagaaataccgccaggtagagcttcggcacaggacggactgttccaatattaagagcgaaggacggatgaatgtcatgagggtgaaagaagacagtgttggaaagatcatcgaggaagcaaaaggcagactgtctgacattacagaggatcgcacaaagtacaccgagatactcgaaaaactcattttccaaggtgtcctcaagttactggaacccactgttctgattcgatgtaaagaaaacgacctgtgcattgtgaggcaactactgccgctagtagctagggactttgagaatattacgggatccaatcttacgctgctgatcgattctgaacagtacttaccacctgagatttcaggaggcgtagagcttatcacccccgatgagaagatcagagtgaacaatacgttggaaatgcgactagaactgatggccctacaagctctgcccgatatgagagatatattgtatggtcctaatccgaataggaagttcgacgattga

SEQ ID NO: 35 - RNA, 1131 nt, synthetic construct
  source 1..1131: mol_type = other RNA
atgtgtgacgatgatgtagccgctttggtcgtggacaatggttccggtatgtgcaaggccggattcgccggagatgacgctccccgagctgtcttcccctcaatcgtcggtagaccccgtcatcagggtgtcatggtgggtatgggtcaaaaagactcctacgtcggtgatgaggctcaatccaagagaggtatcctcaccctgaaataccccattgagcacggtatcatcaccaactgggacgatatggaaaagatctggcatcacaccttctacaatgagctccgtgttgcccccgaggagcaccccatcctgctgaccgaagctcccctcaaccccaaggccaacagagaaaagatgacccagatcatgtttgaaaccttcaacacccccgccatgtatgttgccatccaggctgtgctctccctgtacgcttccggtcgtaccactggtatcgtgcttgactccggagatggtgtctcccacaccgtccccatctatgaaggttacgcccttccccacgccatcctccgtctggatctggctggtcgtgacttgaccgactacctgatgaagatcctcaccgagagaggttactctttcaccaccaccgctgagcgtgaaatcgttcgtgacatcaaggagaagctgtgctacgtcgccctggactttgaacaggaaatggccaccgctgctgcctccacctctctggagaaatcctacgagcttcccgacggtcaagtcatcaccatcggaaacgaaagattccgttgtcccgaagctctgttccaaccttccttcctgggtatggaatcttgcggtatccacgagaccgtgtacaactccatcatgaagtgcgatgttgatatcagaaaggacctgtacgccaacactgtcctgtctggaggtaccaccatgtaccccggtattgccgacagaatgcaaaaggaaatcactgccctggctccctctaccatcaagatcaagatcattgctccccctgagagaaagtactccgtgtggattggaggttccatcttggcttctctgtctaccttccaacagatgtggatcagcaaacaagaatacgacgagtccggccctggaatcgttcaccgcaaatgtttctaa

SEQ ID NO: 36 - RNA, 2247 nt, synthetic construct
  source 1..2247: mol_type = other RNA
atgggtgatcaagaaatgccaaattatataaagtttttgttcggaggaactgcagggatggcagccacagtattcgttcagcccttggacttgatcaagaatcgaatgcagctggacaaagctaaagaataccggagcagtatccaagcattcacaaccatcctgaggaaagaaggagtgtttgccatgtacaatggtttgtctgcaggtcttctgagacaggcaacttacaccactacaagactgggaacctataatttactgctaaacaagttcaaagcggaagacggaaccccgcccaatttctttcaaaaagctgcgttcggcattgctgccggcgccacaggggctttcgtaggcacgcccgctgaggtagcccttatccgtatgactgcagacggtagactccctccagccgaaagacgtaactacaagaacgtattccacgccctgtacaggatgacatcgcaagaaggtgttctcacactgtggcgaggaacaatacctaccatgggaagagctatggtggtcaatgcagctcaactagcttcgtattctcaggccaaacagtttttgttgagcttgagctatttcgaagagaacattttctgttttttcgtgagcagtatgatatctgccttcatcactacggtagcttctatgccagtggacattgccaagaccaggattcaaaacatgaaaacgatcgacggcaagccagaattcaaaggagcgttcgacgtgctcggcaaagtcataaaacaagaaggtgtccttgccctgtggaaaggtttcatgccctacttccttcgaataggtccgcacacggtactgacctttatattcctcgaacaaatgtgcatcttctataacaaaaagtttatgggaaacacctcaggcaaggggagcggtatactgaaaccagaatcagtggatattacaatagccgattttgatggggtactatttcacatttcaaacttaagtggtgataaaacaaaaataaggactagtatatcactcaagttctacaaagagctagaggaacatggtgctgatgaattgttgaggagagagtatggatcattgctgtctgaaccagagccaggttacaatgtgtccgtactcatcaacctggaagatgtgccctcaaattgggaggagatagtcaagaaaataggtctcttaaaaagaaattgttttgcatctgtgtttgaaaaatattttgactttcaagagagaggggaagaaggtcacagtagagcagtcatcaattatagaaatgaagaaaccatgtacgtagaggcgaaggcagatcgcgtgacggtcgtattcagtacggtattccgagacgaagatgacgtcatcataggcaaagtattcatgcaggagttcaaggaggggcgacgcgccagtcacacggcccctcaggtgctgtttagtcacaaggagcccccacttgagttgcgaaacacagatgccagacagggggacaatattggctacattacgtttgtgctgtttccaagacatacgaatcgggtggctcgagagaacaccatcaatctgattcacatgttccgagactatttgcactatcacatcaaatgttccaaagcttacattcactcccggatgcgagcgaaaacgtccgacttcctcaaagtgctcaatcgcgcccgacccgaagtcaaaaacacagagaagaagacaatcacttatcagatgatgaagtcactgccatgtttagaacaactctgccctaggtgccacccctactccatcttgtgctcaatgtcactggagtcagcacctggacttcttggaatggctaactgcacgaaatcaatattggacccattgatgtgtggtaagatggaggtggacagtgctatcgatttagagatagatgagattctagaaaagcctctgtttggggtcagagtgtgggaaatattgtacggagctttcgccgtcatttccctcatagctttggttcgagtcaaaggagtgtgtgatgctgaaaaccagcccagtccgggaggttctagtcagtgtgaagacctttgtgatgataatcatgatacccggagagacgaacaaatgaagctgcacttgagaaaactggatcagaacaagcacaagttcaagttttccaacatgatcaaatctctgaatgtattcccgtcacgcatgcgacattcagcacctgctctaaatgtcaagccaagggaattgcaagtgtaa

SEQ ID NO: 37 - RNA, 1380 nt, synthetic construct
  source 1..1380: mol_type = other RNA
atgcctactaatgcacctcctgaacaaaaaacatctagtctcttaactcggcaagaaaatgaggctgtgttcaagttgttgggtaacagatgtcaggcactctcaactactgtgatacaactgttcactaccgatggaccaaatgataacgagtggcacaagagatgctttggtatcctatgtcttgtcaaagataacccaaggaaatcctacttcttccgtttatactgtctgactagaaaacagttgctgtgggaacatgagctttatacaggtatggattacatggcacctcagaatttcctgcacatatttgaagctgaggattgtgttgtggcattcaactttgcaagtgaagaggaagcccgccacatgagatatgtgatattggagaagcagaagaggattgagcgtagacacagggcctctactcagccgagacatgctacccccggggcagatcgcgagagaagtagaagattacagcctgctgttcttaccaatggaactaacctcacacccaagtctagacatatgagatctttatccggtggcagaggcggtgatagaaggcgagatgggaagaagaataaacttaccaaggcagacatatcatcacccactgagttccggcatgtatcacacgttggatttgatcccaatagaggatttgatgctgttgacattcagaatagtcccgaactagaaatgttctttgaaaaggcaggcgtctcacaaagtcagctgcaagaccggaagacccgagagtttatttatgatttcatatctcgtaatggcggtctggacgctgctaaggaagaggcgcaccttcccccaccccccgtacctgctcgctctgtccacccgtccaaccctcgccatgccccccctccacccccgtcccgtaatataccgcctcccctgccatccctccgtaccctcccctcccgcgcccctaccgcgccacccaaagacatgcacataccccctcccccgcctccacccccctctctaccaccccctgctaccatacagcagagtacaccccagcctgcccagcaacctgatcctagagctgctctcatggacgccatacgatatggaaaacaactcaagcctgtagatccagattctaagaagtctactctgaccaatgattcgagaggagaactgttagatcaaatccgacagggagtcgaactgaaagcggttcaacccgtatccaagccagcagtgagtacaccatctgatggtttagccggagctctagcgcgcgccctggcggagagatctcgagttattcatagtgattccgatgacacaagtgatacagatgaggaaaatgaggatgaaaacgacgaggatgaatgggaagactga

SEQ ID NO: 38 - RNA, 843 nt, synthetic construct
  source 1..843: mol_type = other RNA
atgtggaaagcagctgcaggttctggagtagctcccgttgttgcaaatcaagatgatgatgactgggaaactgatccagactttattaatgatgtgagtgaacaagaacaaagatggggttcaaaaacaataccaggctcaggtagaacaggaggtactattgatatgaaacagttgcgagaagaagttgccatatcagatgcttgctacaaacagaaacagttggaggagggatctaaagcatcttatggatatggtggtaaatttggtgttgagaaggacaggatggatgagtctgctgtcggacatgactatgtcgctcagcttcatcaacatgagtctcagagtgattacaaaactggattcggaggaaaatttggagtgcagaatgatagagtggacaaaagtgctgttacttgggatcataaagaagtaatagagaaacatgcttctcaaaaagattacagctcaggctttggggggaaatttggagtacaaaaagatagacaggataaatcagctgtgggatgggatcatcaagagaaaatagaaaagcatgaatctcagaaagattacgctaaaggttttggaggtaaatttggcattgaatcagacagacaagacaaatcagctgttggttgggatcatgtagagaaagtagacaagcatcaaagccaaaaagatgacaacagacaagttgtgacttcatctaaagtaaaggacctaatagcagctaattccacaaatgcttccaaggaaaatattaaaccgaaaccaaatattggtcatgttaaaccttctaacctgagggcaaatgaaatcaatcctcaaacaaccaactag

SEQ ID NO: 39 - RNA, 8469 nt, synthetic construct
  source 1..8469: mol_type = other RNA
atggcttgcgatcgttgtgttcaactttcatcggaagttgaaaaattgaccactcaaaataagaagattgtacaattgctgaatgtgttaaatgcctccaagaaacacaaagaacaactgatagaacataccttggattacatcaagtccaaagatgaagactacaagaaggaaaagatcaaaatcaaagaacttgcaatcaaagtcaagaacaccaccaaagaatcggttctcaggaatgtacccaagcagattgatgagtgcagtgcactctacatcaataaaactgctgagatacagaattacattagagccacagagattgccagagctgccaagcaagagttggaaacaaagagccttgaattggaaggaaacaaaatgtacgtgaagcaactgattgccaaagttgatgagctagaacataacaagaggactttgacggaatccttggcatcaactaaactgctcgtcacaaaagctttgatgagaaacaagaagatagagcttgagatgagaatgctcaatgatgctcactcatatcataacagagtggaaaccggagtgaagaaagctttgcgtagattggaatcatcagatccaaattccacaaagagctgtatgaggatcctgaataatctagttcatttctttgaaaaagaaccactctttgtgaaacgatctcggaaattgaagaatggtgcggataataagggcatagtcagtcctcacagtgtatcttctggttatagcagtggaactgcaagtccttggtctactccttacaatactcctcaatcctctgaggaacctaacactcagttcacatttaccttcacaccggacggggattgtgtgctctcccctcttccggctcagtcacagtcaaacccttcaacttcatcctcatcaacccccaccaccaggtcttccattacttcaccattggttcaaccatctccacctcattccagctatgaaccaagtcagaaacttagttctgagaaagtcgcaacctcttggtcaatgtcttgtgatagttcggatgatgaaattgttgatggagctgatgatcgggatagtgatgttgaaagtgatcgagagacctctataaaaaacacttgtaaattagaaaccaataaacacaaaagtaaagaccttggtgatttagcaagtgacaaagtcaaggaaactagggaattacaaagtgataaaggcaagtcgtcaccaaaaagaggtgaacttgacagtgatatggttgaaattggtgagcggagcaagaaaccatccagtgataatgtacaaagtacatttgatcttgttgagccaaatgacatgtgtgaaagtaacaaaatcgattgtgataatagtgatgaagggaagagcagtgatatccaggagataactatagcaagtgaagtagccagtcccaaacatgcttctgcagagttactacaccttcctgtccttgagaattttgaagatgaaaaacctataaaacaattacctggagttacaagtgcagaaaaggatcaagcccttccaaagcaaactgttcaagctaatcatagtccagaagctaaaaaacatttctttggttcgtataagcagagtttgttttcaccttcaaagacaccaccgggaaagaaggaaacaaaacaggagattgcaagggaaattgccaaagcggccaaacaaatagaaatgagtctctgtgaagaagagaattgtgtgaaggagaagaagaaacgtgtatcacgaagtgtgaagagaaggagctctaaaaagtctataggcgatgatgaaactgacgcagaaagtgctactgtggttgcaaaagaaaagaaaaatggaacgtctgaaatgagctcagaagatgacagcttggaaaagttgcgtgtgttgagaaaatctcgtaaatcaattgacaaagctctcaaggataatgtcggtagtactaagtcttctggaaaatcgccaagacgtagatcaactagaaacaaaagtacagaaaccagttctattcgttcagaaactagctgtgtcaattcagaaaacaatcttattgactctaccagcacagactttgaaatttccaacctctcagacagtgattgtgaaagtactgatcttgtagacaaagctgatgagaacaccgaagaaaatgtcaaggaacttttgaaaatcgacaaggaacgacacgaacacgtgttacagatattcaaagacattgaagtgatgcaaaatgttgaactcatttcgaactttgatcttctggcttcaccctgcaagaaacaaacgcgatccagatccaacagtctagttccaaataacgaagcagataaactagaaaatgatgtaattcctaatgcatttgattgtgataaagatatcagcaatacagaacttgagaaagagaaatatgaatcactcgaagctgtatataacgaaaccaacaaaaagctcgtgaaagattctgaagaaaatgcccaagatgatgaagatgattgtaattcacaagtagaagatgatgaatgcaaaacgccctctttgagaaggagtagaagaaactcaaaacggaatgcagaagagatatccagtcctgtaactaggaagtcacctcagacccaaaacaccgagtccaatttgaaaggtaccccctctgacaacaagaagtcacgaaaatcacgcaccatcgaagaagaaaggttaccaagaacgagatcaaaatccacgcacaaggcagtatctattcctgaagtagataatttgagacaaagaagctcaagtgacgttggaacaaagaagtcctctgaagaaacggaaataccacttgccgatagtgatgaaatacctagaagagtaacacgaagaagtctatccgtcatggagaacaaagaagatgttaaatattcagaaaaaacaagaagaggacgtaataagaaggcggacaatagtttattaggagaggcaatgtcagatggtgacaaggataagattagtttcgaagataagtgtgataaaaacagtaaagacaaaccgaattctagtgactataatggaaaagatcaagttgttccaagtgctagcaatgaaattgaaagtgttgatgatcttgatgaatctatggaaataaacgatattgatgcttctgttgaaacattatcagagataactgataaatctagtggaatagacacagagaaaaatctcgtgaaacaaatagaacctgagaaagaagaaatggtagacgacgacaaatcaagtaatcttgaaagaactctattcgaaagtcgtgtagaaattacagaaagcaataaagaagatgtagaatgcaatgacagcatggtacaagatgtgcgatgtttgaagagaactcgaattgccctatctaaagtcggtggtgatgccgtgtccaaagtatccagtgatacacttgatacagtatccaaagtcagtgatgaaaacaaggcgctgtctcctccagagaaggctgttagctatcctttggaatcgaaggaaacaccagataaatctgctcccaacactgctccatcgacagctcaacctatgtcacctcagaatgataaaaaattagaaacagatgccccgaaaatacctaaaccagtttcagttcttgaggaacaatcttcgaagatatctgatccaagtcctgttctgcagttaagtaccgggagaaagttcaagattggacgcttgaaagaggtatctaacactaccaatgaaacagcagtccctaggaaagaattggccgatagcagaaatattgagactagtgttgaatccagtaaggaatcgccaagtactcccagtagtggaaaagaacatgtggaactagcaaatgaaaaagttattgttggtaaagaagatcaaatccacaagatcagatcgaacaaaaacattttgaatactaaaaatgagaaagtacttgctaataaaaaggatacagtccccgaggatgtctctacaaatgaaaatggccaaaacattctaaaccatacagtcaataaagcttcgacaaagaatactatactacctattgtcaaaactgacaacttaatgcaacaaaacagaatcatccaaagttacaattgtaacttgaacctggtcaaaccagccacagtacagaaacctcctcatacaatcgtgaatagtaagaaagtcaatagaattctccgaagaatgggaatggagggattcaacaagaagtttccattcattgacgctctccctgctactccacctcagtgtagagaaaatggaactggattcaagaactacaatgggtataacaactctactattgcgaaggaagaagttgaagagatggaagaaacagattgtttggttatggagatggaagatgacttacaagatgttagggaaccaaaagatgcagatagtgaagtatctgaacaattgagctcgcaaactacatgtgaagataaagtacctgttccagatatgacacaaccacttgcagataaagaagaagtagtagaaaaatcgatcagcttgcctaggaaagagggagaagaattaaaccaaatgccagtacctattccgaatataacagatatgacacagccacttgtggagaaagaaggaggagtggaaaagtcacccaacagatctcaaagaaaggatgaaaaattaaacttcgtcagcaaaataatggaaaacagtcttcacaaagaagatcaaatccaaaacaaaggaattgttcctgagcaaagcacaagcttccctaatgtacacgtcccagacaaagagtctactagtcccaataaaatgaataatgacttgagtcctaacaaatcagatgacacaaccagcttgacatccacaacatctagtggattgaagtctccttcaaacctgttgacatccaatgaaacaccttgccaatctttgcagtcacctccctcaatatttcccaaatgtattcaaaaccgcttggcaaagatccgttccacacacaatttaccaatttataacataactccaatgagtaatttggctcacaatactcgacctggggagaaaaccaacatgaatttaggtgttcatggactcaagtcgaatgaaatgagccctccaagtctaaacaaagtagacattcaagatgccgtcaaaccagttcaagctagtcctacagatatttccaatcaagtgtcctataacaataatccagaaccgactcttgttggaaaccaaacttttagcttctatggacagaagagatcttcggacattactgctccttttgaaaaagaaagcacggtatttagaccaccaaataacttcagtaatactaacacggtttctagaccatcaaataatgtcacctctaatagtgacaatatggtatctagaccaccaaataacttgaacactagtttcgtcaacaatagccctcctaaggtcaacggacctttggagaattctgttcatcaggcgaatgctaccaaacccacggaaaagattgttcttccaacattatccttctacaacacacgcaattctccttcatatggtgctaacaaacgtaaatgtgaagaccctgaatatgtgagttctgatgaaagcaagaaacttagaactgagcttgaaaccagtggtacattgaagagcacggttaacacacctaaagacaaagatgaacccaagtcctatacactcattcaacgctcaattgaacatctacaatccgaaatgtccaaacccgatgtgacaccaaccaatcagatcaaaactaaaacggtgacgaaaaaactgaacctgctgtactacgccatgcaaccgaatgataagaacgtgttcgataggaaaatcaaagcgtttgtcaaagagctggaagattacccgctggatactttatccaaacgaatcatcgcgttgtacgataacaaacagctggtagacaatcccaaagcggtgacaaacattcaaaccaaagtgttctgtctgattatacagctaaacacaacacggcctgagttcctacccacgtttatgaagaccctgcatgagacactggtcctgcatagtgagaccacactcctatctatcctgtatgcattgacacaactctatgtaggcttgtgtcgctggcgtaatgatccagcatctgttatgacattcctctacgataccatctacttccataggaaacgctctctcaatatactagacgctttgatcacacgatggcctactgtgtatcctgtagccagtcgctgtgtcctagttaaagtaatgagttgtatcctgctcaaccagcagacatcaagccaggggagtttccttgatgaccagaatgtcttcgtgaagacaaagcagcagttgagaacaaagtacagatatcaggaacagttcacacttgccggtctactagcagagttgatgaatatgttgaagagtaaacactcctgctgctctaacccgtggcagcgaaaccctcgttccaattctcaccaccagggagcggaatcgtcgagtttcagcggttgtgtacagagggcgctggtgctgttggctaaacattggcgtccggcgccagacttgtacagtgttgtcgtggagaggaacttggcaacactgttcagaagtagtttggattgtggagcaatcagtgttgctgcctcctgcgctgaactcatgggtatgattgggaaagccatgccccgcgcctcctccctcctctgcatcgtctccctgctggatattctggaggtcggcctgcaacatgccgcctgccgctcaccctccccccttcgcacctctataggccgcgcgctaacctgcctcacggcccacaacacggagcgttgcgtggccatcctcacctcggtgccttggacagaggccgaggtcaatgaccttgtcaaggacgttgcagccaaggtcatcgcgtcttgtaagatccgagtgacaagcaccatgatttccgaatctctcctagaacacctgctcaagaagtaccgctggattttcgtcattttcttccttctgcccctgactttcttctacgatatctatcacttcatacgacagcaggtcaccgaatatttcaaggacaaatcagtatgccatgatctcaaagtaaaacatgttcaaggtcaggttcgtgagtggatcaaaaccgaccaatccataccgatgtgtacaggacgcgctgggtggaagtgtatgtcactgagagagcccaagtacaagtcgtccatgtttccagtggatctggaggcgatggataccatcctgagtgtggacgaggagaagaagacggtcaaagtagagccctatgtgaccatgggtcaattaacccgctatctaatacccagaggctggaccatccctgtcgtggtagaactcgatgacgttacagtaggaggcattgttctgggtcaaggtctggagtcaagttctcacaagtacggggtgtttcagaacacatgtgtctcctatgaactcgtactgagtgatgccagtctggttgaatgtagtgaggaggaagaccgggatttgtttcacgctataccttggtcctatggaacactgggatttctgacggctgttgaaattcaaatcattcctgtaaaaaaatatgttcagcttcaatatgtcgcgttgaaatctctgccagatctagagcatcatctgaagaaggaagcggaaaacaaggccaatgatttcgtggaagccatcttgttttccaaggaccagtccgtgctcatgataggtaccttcagtgattcgccagatccaagtaaagtgaacaacctaggccgctggtataaaccctggttctatgaacatgtaaggagctaccttaccaagaagaagttctctgaggaacatgtgcctatcctggattactaccatcggttcagcacgtctttattctgggaaatacaagatatcgtcccatttggtaaccatcctctatttcggtatctcctcggctggctgatgccccccaaagtggccctcctcaagctgacccaaacccagaccattaagaacctctatgataaacatcacgtagtacaggactacttggttcctatcgaggagcttagatcatgtgtacactactttcatgacaatattcagatttaccccctctggatttgccctttcctgctgaaagacctgcctggtttggttcatcctgctaaggcacaggatggtatgtatctagacttgggactgtatggagagcccaaagctaaagactaccacagcaagaacacaatcaccgccttggaaagctatctgggaaaaatcagagggttccaaatgctaggcgcgggtgtctaccaatcatattccgagtttcgacagaactatgaccatagcttatacgacagagtacgagccagactgggctgtgaaaagggttttcccgtcatctatgataaggtcaatagagttgcccgggactga

SEQ ID NO: 40 - RNA, 399 nt, synthetic construct
  source 1..399: mol_type = other RNA
ttttccactgtaaggaatattcacgcagcgcccatcatgtccactgaactgccattgatgaaacggacactcaatacaatcccctctcacgaatcctccgatggcaatgttagctcccaaatgaggacagtacgcatccagcacatgcacttctcccctctccgatcggaacacagcaaagttttccccgagtgcggaaacatatttggcctgtttgggtttcagctccgaggattctagcagtgcaaaccacccgtttggatacacgggcgggaggttgcctattttgcgcgccttgcgaatgtcgttgatgagctgttttttcgttttgccgtacttgttgtagccagaccctcgtccgtccaagtaactgtagccaacttcactcagatcctgtaa

SEQ ID NO: 41 - RNA, 579 nt, synthetic construct
  source 1..579: mol_type = other RNA
atgcaagccataaagtgtgtggttgtaggggatggtgccgtgggtaagacctgtctgctgatcagttacacaaccaatgcattccccggcgagtacattcccactgtgttcgataactattccgccaatgtcatggtcgatggtaagccgatcaacctgggcctctgggatacggcaggacaggaagactacgatcgactccggcccctctcctatcctcagactgacgtgtttcaaatctgtttctccctggtgaacccggcctcattcgagaacgtgcgcgccaagtggtaccctgaggtccgtcaccattgtccaagcactcccatcatcctggtgggaactaaactggatctacgcgacgacaaggaaaccattgagaaactcaaggagaagaaactggcacctatcacatatccccagggtctatcaatggcgaaagagataggcgccgtgaagtatctcgagtgcagcgccctcacacagaaaggtctgaaaactgtgttcgatgaggccatccgagccgtactctgtcctgtgcctactgtgcccaaaaagaaacgctgcgtgctcctgtaa

SEQ ID NO: 42 - RNA, 1305 nt, synthetic construct
  source 1..1305: mol_type = other RNA
atggcttcttcgggaatatggagtctgaaaggtaccccaggtatcttggaggtctataattatgtagactccacattagaagacaagggagatgaaggcagcaccaccaagagagcagaggttgcctccaaacttctagaaacacttgagaagttcaactacgatgaaacaacagaacccgcagattcttacatctccgactggctcggtggcgaagagaaggtcatcgaccttcccatctacgaggacttcccactgtcccaaccttccatcgttcaaccaggtagcgctaccgtcgttcaaccaaatagcgctagcatcgttcctcaaggtagcgccagtgtcgcgttccgtcaaggtcacgcgggcttcgctcaaggtcacggccaaggtcatgtgaaggtcggctacgaggcgagccccgtgtggcaggccgatgagtactcgagcctgtattcgcctccggccgcgcaatggccggtgtataacgagatggtggccgcggtgtcgagtcagctgaccccgccccacagtcccaacatgtacgagctgagccccaaacacgcggccgagtttaaagatctggccgatgacttgatgaaacctgcggccggcaaacttccgccctacccgtccgatatcggcctccaggccaccagcccctgtctcctcccccctcaacctgactacgaggagaatctgaaggacggaggtcagctcattctgtctctcctggccgagatgaatcccaaggacatcagtgagctggtccaagccaacgagcttattcaagaccaaggtcaaacaagcgagccctacaccgtccccgatctgagcgctccagcgagcagcggcgcaaacatgaactacagcgaggtcatccttagtccggaccactcgtgttcttccgactccaacttcgactacacctcggacacctcttccgacccggactatattccgtcggcgcgtgtcgggttccctcggaaaccgagagctcccgaagtagcttcggggaagatcggctccgctcggaaggaaacgaaacgggccaagccgtatgcgcgtaaggcggcgctacctattgaagacaagcgcctgcgcaagaaggagcagaataagaacgcggcgacgcgttataggattaagaagaaggcggagattgaggaggttctgggagaggagaaagagctgttggagaagaacgcccagctgcagaagagtgtggaagatctgagccgtgagatcaagtttatgaagaaattcatgcgggacttcttcaagaaacagggcgtgctcaaatga

SEQ ID NO: 43 - RNA, 2970 nt, synthetic construct
  source 1..2970: mol_type = other RNA
atgatattgtggaaagatggaggagcagtcagtaaaatatcgttccttctgttagccattgtattcttagcttcctctacatacagcacacaaaccgccaggaggcgactgcgacggccctccatcaagtccaaaccgtccgaagactccggggaccaagatgtctctgccagcatcaacaggttcaagctcaagaacagaacggccgcctcttcggccaacagcgccgtgatcgctgccggaagcagcaagtccagtgtaaaagccagcaaagatgaccccaagtataaggtcgtctgttactacacgaactggtcccagtaccggaccaagatcggcaaattccagccagaagacatcgagcccgacctttgcacgcatatcattttcgctttcggatggctaaagaaaggcaagctgtccagtttcgaatcgaacgacgagaccaaagacggcaaggtgggactgtacgagagaatcgagcagctgaagaaggccaaccccaaactgaagactctgctagccataggaggatggtcgttcggaacccagaagttcaaggagatgtccaagagccgttactctcgtcagacgttcatctactcggccgttcccttcctgagacagagaaactttgacggtctggatatcgattgggaataccctgccggcactgacgacaagaaaaactatgtgctcctattgaaagaactgagagaagcctttgaagccgaagcccaagaagtcaaaaagccccgcctccttctgacagctgctgtacctgtgggtcctgacaatatcaaaggtggatatgatgtgcccgcagtcgcaggctacctggacttcatcaacttgatggcgtacgacttccacggcaagtgggaaagccaggtcggccacaacgcccccctccgctcgcccagctctgactccgagtggcgtaagcagctcagcgtggagaacgccgccaacatgtgggtccgtctgggtgctcccaaggagaagctgatcatcggtatgcccacttatggacggacgtttaccctgaccaatccggcggtgttcaaggtcaacagtcccgcgagcggcggaggaaaagctggagagtacaccaaggagagtggattcctcgcctactacgaagtgtgtgaaatgttgcgcaacggtgccggttacgtttgggacgacgagatgaaggtcccctacctggtacacggggatcaatgggttggctttgatgatgagaagtccattcggtacaagatgaactggttgaaggacaatggttatggaggtgccatggtatggactgtggatatggacgacttcaccggaactgtttgtggaggggacgtgaaatatccgctcattggagcaatgagagaagagttgaacggcatcccccgagcaaacaacgccaaagacgtagactgggccaaggtagctggcaatgtggaagtggagaccgtcaccaaaccagcccccatcaagattgatgtgtcagaactgctgtcgaagaagaaaacgactaccattcagaaacaggagactagtaacctgattgacaccaaagttcgtccggcccagatcttctgttacatgaccagctggtctcaggccagacccggcgctggcaaattcggtccagagaacatcgaccctaaactgtgtacccacgtgatctacgcctttggaaccctgaaggatcacaagttgaccttgaacgaagataaggagaaggatgaggctggcaaattcgaagccctgatgcaactgagagagaagaaccctgacatcaagatcctgttagccatcggaggatgggcattcggctccaccccattcaaagaactgactggcaatgtgttccgtatgaaccagtttgtgtacgaatcaattgagttcctccgagaacacaagttcaacggattggatgtcgactgggaatatccaagaggtgcagacgaccgggcctcctacgtgaacctgctcaaggaactgcggctagcattcgaaggagaggccaagacctctggcgagcctcgtctcctcctgacagcggccgtacctgccagttttgaggccattgcggccggatatgatgtgcccgagatttccaaatacctggacttcatcaatgtgatgacctacgatttccacggccagtgggagagacaggtcggccacaatagtccgctgaaaccacttgaaggtgccaccagttaccagaagaagctcactgtcgagtacagtgccaaggaatgggttaagcaaggtgcccccaaggaaaagctgatgattggtatgcctacgtacgggcgtagtttcactttggtcgatccgactaagttcgacattggagctccagccagtggcggtggggagcctggaaaatacacggccgaagctggatttatgtcctactatgaagtgtgcgatttcttgaagaaggacaacacgaccctggtctgggacaatgagcagcaagttccgttcgcctacaaagacgatcagtgggtcggatttgatgacgagagaagtttgaaaatgaaaatggactggctcaaggaagatggctacggaggcatcatgatctggagtgtggatatggatgacttccgaggacaatgcggctcgggcaaataccctctgatcagcaccatgaatactgagctggaggactacacggtggctcttgaatacgacggaccctatgaaagtttcagccccagcggaaagtataccaccaaagaacccaacgtagtttcctgtgaggaggaagacggccacatttcctaccacccggacaaggcggactgcacccactactacatgtgcgagggcgagaggaagcaccacatgccctgtcccagcaatctggtgttcaacccgaaggagaacgtctgtgattggcccgagaacgtcgaaacctgtttgcatcacacccaggcgcctccaccgtccaaataa

SEQ ID NO: 44 - RNA, 4742 nt, synthetic construct
  source 1..4742: mol_type = other RNA
atgagtaccctcagacaacttaacatgcaggattcggaccctgatgacttctcagacgatgaaagttcacccctgtctcccaatgatatctatggcggaagcacccgtacagttcccgaaacgaaagcatgggacgtgttccgagacctcccgcctcgacaggacagtggctcgatggcatcaaaagcttgtctcgaattcttcatcaaggcgctcaaagtgtttgtgtatctgtttacgttcatagtggttctgttgtccggggtagtctctaagactgccgtgttcttcatgacgtcacaactcagagccccggacaagcacatcccctattgtaacaaggagtttggtcgagacaagcagttcgcggtgtatctcccaatcgaggagcgtatcgcctggttctggtgtctcatagccgccttcatcgttcccgaactaggcacggctatccggtccatccgaatctgctacttcaagtcctggaagaagccgcccctgcacgattttatcttcgtcacctccatggagctgttgcataccctgggactggcctgtctggtgtttgtagtactgcccaacatcgatgtggtgaaaggagctatgcttaccaactgtttgtgttttctgcctgccttcctcaccctgatatctcggaacaccaggggctgtacgaagggttcagagaggtccgaagtatacctgaaggccatgatggacatgatagctgtatctgcccaggctacgggggccctcttgtggcccctgttggaggggcacaatgacccctggctgtggctgataccgccggccctgttctgtgtgtcctgcggttattgggagaactacacgagcaaacattcaatattcggtttcatgaagtcagcatggcgggttaaggagagactgaagcgtacccgatacttcacctatgccttcgtgtcattcctcaagatcctggtattcctgtgttctgctctcgtattcatgtcattccgcggagaaagcgttacggaatacttctccaagttccacgactccttctcacagcacaagattcgaatttatgaggtgaaagcttcggcatttggcacttctatcccagacttggctgatcctaatcttacaggcgattacagggatgtagattccgaggataatttcgccatgaagatcctgcttttccagatctttgctgcatatctgtgctacattttcggcaagtacgcgtgtaagatcatgatccagggtttcagctacgcgttcccggtcaacctgaccattccagtcaccatttccattctgatcacaatgtgtggactgaggaatgatgatccgtgcttcttccagaactccatcccggactatttgttcttcgtgtccccctctatctatttcctcgacgatttcatattgaaacagcacgcctgggtttggttgctatggttactgagtcagacttggattacgcttcacatttggacccccaagtgtgagaggttagccaccactgagaagttattcgtacgtcctatgtatgacgcactgctcattgatcagtccatgagtttaaataggagatgcgatgatgagaaagacgtcaagacagaggaactcgtagatcgagagaaagatccagacgagtactacgagacgatttccgttcacacggacgcctcgtccacaacgcccaagaccgtgaagaaatccgactccattacgcgcatctacgcatgcgcaaccatgtggcacgaaacgaaagacgagatgatggagctggtcaagtctattctgcgtttggatgaggaccagtgcgcacggcgcgttgcgcagaaatatctgcgcgtggtcgatccggattattatgagtatgaaacgcacatctttttcgacgacgcgttcgaaatttccgacgtgaatgacgattggtcgcaagtgaatcggttcgtcaagctattcgtatccgtcattgacgatgcggcggcgcatgtgcacgagaccaacattcgcatccggccccctaccaagtatccgacaccgtatgggggtcagctcgtgtggactctgcctgggaagaccaagatgattgtgcacctgaaggacaaggccaagatccggcataggaaacgatggagtcaggtgatgtacatgtactacctgctgggtcaccgcctcatggagttacccctaccggtagacaggaaggaggttatggccgagaacacttacctactcaccttagacggagatgtggactttcaaccggacgctgtacgactactggtagatctgatgaagaagaatagaaacctgggtgccgcgtgtggacgtattcatcctgtgggaggaggtccaatggcttggtaccagaagttcgaatacgccatcggtcattggctgcagaaagctaccgagcatatgattggctgtgtactttgtagtcctggatgtttctctctgttcagagccaaagctctcatggatgataatgtaatgagacgctacactaccaaatcagctgaggccagacattacgtgcagtacgatcaaggtgaggatcgttggctctgtacactcctactacagcgaggctatcgagtagaatattccgccgccagtgacgcttacaccagatgtcctgagagtttcaatgagttctacaatcagcggcggcgatgggcgccctctactatggctaacattatggacctgctcatggactataagagaacgatcaagatcaacgataacatttccatgccctacatttggtaccaggtgatgttgatgggaggtaccatcctaggtccgggcactatattcctcatgttggtgggtgcctgtgtggccgtattccacattgataactggaccagcttctactacaacattacgcccattctcttcttcatgtttgtctgcttctgctgcaagtccaacttacagctcctcctggctcaaatcctatcgacagcgtacgccctcctcatgatggccgtcatagtaggtaccgccctacaactcggcgaggacggtattgggtccccgtccgctattttcctcattgtcatgttggggtcgttcatcatagccgcgttactccacccgcaggagttcttctgtattgtattcttctctatatatctcctatcggtgccttccatgtacttgctcctgattctgtactcgcttatcaatctgaacgtggtatcctggggaacgcgtgaggtggctgttaagaaaaccaagaaggaactcgaggaagagaagaaggccgccctgttggccaaaaagaaagccaagaacccgctgctcggatttctgcagaactactcacctgaggctgatggtgatgaagaaggtagtctggagctatcgtttgccggtctgttcagatgtatgttctgtacgtatcccaagccagtggatgagaaacaacaactgcttcgcatagcggacagcttgagttcgttggagaaacgcttggaccatattgaacgagtggtagacccgaacatgcccaccccgcacggacaccgccgacgtactacctcagcctccagtagcgctcaccatgccatgggtctgggttcggttcccgagaataagagcgaagatgcgtccagtaatgaggaagatgaagacagcgatacagtgtccacagcggaacccaaaatcgaacgcaacgacgacattaacccctactggatcgaagacaaggaactgaagaagggacctgttgctttcatttcacccaacgagagagcattctgggttgatttgatagacaagtatctctaccccattgataacgacaaggctgagcagagtcgaatagcgagcgatttgaaggacttacggaccagtccgtgtttagtttcttcatggtgaatgccctgtttgtcgtgatcgtgtttttgcttcaactcaataaagataaattgcacatccgctggccattcggggttaaaacgaacattacgttcaatgagtcgacccaggaggttatcatcaccaaggaatatctacaactggaacccatcggtttggtgtttgtgttcttcttcgccttgatcctggtgattcagtttaccgccatgttgttccacagatttggcaccatctcgcacattttggcctcgacggagcttaatctatgttgcaacaagaagactgaagagctctcccaggacggtctcctagacaagcaagcggtggacatagtccgccacttgcagcgactacgaggcattaacggggagtacgaaaacgacagtggctcctccggcgaccgagtgggcagaagacgaactatatacaatctcgagaagcagaagaacaaaacgcgaaccattggcactctggacgtggcattccgaaaacggttcctccagatgaagatggaggatgctgtagagacagcgggtaccccagtgctcgggcgtaagttaaccatgaggcgagaaatcagagaagctctggaagtgaggagaagatccttacaagccgagagaaggaagtccaacatgcaaacattgggggctcataatgatatttatggccgaactcaaaggatctcaaacgcgggatctcacgtgaaggacattttcgacggccatcagaaccctgcatacgagcctgatctggcctcggattcggacaggcctttgccggccactatggaaaacagggccgtcaattgggccgagcttgaaaatcagcggtcaaatagtcacatttaa

SEQ ID NO: 45 - RNA, 1980 nt, synthetic construct
  source 1..1980: mol_type = other RNA
atggcgtttcctttttacggaagtattgcacttttggcgagtctcattttcatctcgatttctcctacgtctggggatgactcagacgtcccggccaggatatcctgttatttcagtaactgggcaatctacagacccggaattggcaagtacaccatagatgacattcccgcgggtatgtgcactcatttgatctacgcgttcgtgggcatcagcaacagtacgtggactctggaggtgttggatcctgaggttgatgtgaaggagcatgggttccggaatttcaccgctctgaggaagacacatccgggattgaaagtggaggtggcagtaggcggctggggcgaagggggagagaaatattccgccatggtggccgacaaaacgaaacgagccacgtttattagaagcattgtcgctttcatgaagaaatacaacctggatgggtttgatttggactgggagtatcccgccgcttcggacagaggtggcagcttcagcgacaagaacaacttcttcttctttgtggaagagctgaggacggcgttcaacaagtttgggaagcactgggaaattaccatggccgttccgatggccaagtttcgactcaatgaaggctatcatgtgccagacctttgcgaacttgtggacgctattcacgtcatgtcttatgacctccgaggcaactgggcaggatttgccgacgtccatagtccactttacaagagaccttcggatcagtgggcgtacgaaacactcaatgtgaacgacggtcttcagttgtgggtagacaagggatgttctcctcgcaagctagtagtgggggtgccattctatggtcgctcgttcactttgagttccggaaacaacaactacaacattggcacctacattaataaggaggctggaggtggaacaccaggtccctatacaaatgcctctggattcatctcgtattatgagatttgttccatggttcaggaagataaatcctgggttcagaagtgggatgatattggaaaatgcccttacacatatcaaggcacccagtggatcgggtacgaagatccaaagagtatccaaattaagatggactggatcaaatctaaggggtacgctggagccatgacttgggctatagatatggatgatttccaaggactttgtggacccaagaatgccctgatgagcattttgtacgagaacatgaaagactacagggttcctgtgcctcatgtttctattacaccacggcctgaatgggacagacctcccagcactacccccgaggacatagattatgctcccacctttacgttcaccacaaaacccgaccttccatcgaccccaaccacaacagccaccaccgaatcatcaactccagaagtcaccaaacctgtggaaccaccaaccacacccaagtacaaacccaacaagcgaccacgaccttccatcacggccaaacctccgcgcacaactacgggcaaacctgagacgaatcgacctgacagtgctgaatcaaatgagacgaatggaccagacagttctgagacgaatcagccagggagttctgagacgaatcgacccaaacgtccagtgaaaaggccacttcgcccaacagtcaaccgacctgatcgcccaccagtaaaccgacctaatcgacctgagactgttgaagcaaccagtcaagaagttctggagccaagtcctgaggtcactgaggaaaaccggccggaaggtaacctacccgaagagactgaagtggattgttctaatgatggattccagtcgcacgctctgtgcaataagtactaccagtgcgcgttcggcaaaccaatcgaattcacgtgtcgacctggtacctacttcaatcggaaaatgagcgtctgtgattggccagagaaagtcgatgttacccgatgtaaaatggtcaaacccctgaagactgtcgaatatgaggatgagaactacttcctgtga

SEQ ID NO: 46 - protein, 1599 aa, synthetic construct
ATGCCGCTACCCAAGCGAGTGATAGAGCCAGTGCATGTGTCCCGGGGTACAATACCTGAGGAGCTGTCTGCCAGCCTCCCGTCGGAGCTTGAGGCAGCCACCAATGGAACTCTGGCCAACACGGTTCGACAGCTGTCCAGTCTGAGTCGGCATGCGGAGGACATGTTTGGTGAGCTTACCCGTGAGGCGCATAACATGGCCATGAGGGCCAACTCCCTCCAGGTTAGATTGGACCGGTTGGCAGTCAAAGTGACCCAACTGGATAGTACCATCGAAGAAGTGTCGCTGCAAGACATTCACCTGAAGAAGGCATTCAAATCGGCCATCGTGTTTGATCAGGAGGTTGTATCTCGCAGTACCATGCCCACAGCTATGCTCGAGACCTACAAGCAATGTGATAAGCCACCGCCACTCGACAAGTTAGATCCGTACAGAGAGGATGGCAAGTTTGGTTTGAAGTTCTACACAGATCCTGGTTACTTCTTCGACCTATGGAGACAGGAGATGCTCAAGGATACAGAAAGGATGATGCATGATCGCGGGAAAAAGCCACACCGACCCCGCACGACAGAGAGCGGCGGCGGGCGGCGACAGAAGCGCGTCCGGGCGCTTCACAACACGCGGGAGCGCCAACGTCAGCTCGCTATGAGTCACGGCGAGTACATCATGCCTCACCAGGCGATGTATCCCCCGCCCGGCTACTCCGAGGAAGATATGATGATGGACGGGGGCAGACCGTCGCGGCCCGACAGCATTGACCTGTCGGGCCGACACTATGTGGACGGCGCGGGCATGCCCGACGGCGGCATATACAGACCCGGTCTGCAGCAAGGCTACATGAACTACGACCAGGTGATCTACCAGCATGGCTCCCAGGGCGGTCACTACGGTACTGGTATGCTTCACTACGATATGAACGCTATGGGCAACAACGTGAACAATAGTAATCTAGAAACCTACACATCCCCTGGTGGCACTCCGAACCGAACCAAACCACGACCTTCCCAGCCTCCCCCTGCCCCGCCCAGCACTACCAATTCCAATAATAGTACCCCCACCACGGTATCCGCGAGCAACACTCCCACCCGGGCCCGCAGCATGTCTGCCGGCCGCGACATTCTCNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNNTGCCCCCACTGATGAACGGAGACCTGCACCTGCCCAACAGTACCAAGTCCCCTCTCAAACAGGGGTCGACTACTTCGCCGCCAAAGCCCAGTAACATAGACGTACGCAAACCCGTGTATGATCCTAGGAGTGATCTGCTCAAAGCTATACGAGACGGCATCACGCTACGCAAGGTAGAGCGCGTAGAGCAGAAGGAAGGCGAGCGGCAGAAAGCGCTGCATGACGTAGCATCCATTCTGGCGCGCCGAGTAGCGGTCGAATTCTCCGACTCTGACTCCGCCAGTGAGAGTGAATACGATAGCGACGGGTGGCAAGAACAGGAGACCAGTGCTTAG

SEQ ID NO: 47 - RNA, 2553 nt, synthetic construct
  source 1..2553: mol_type = other RNA
atggaaaccgaagaaataaccaaactggttgattccatttacaagaatatcagcgacaagttcaatcctggagctcgtcagatgatcagtgctgggaaagcttatctcaaagctttacatggagccgcagctgcctctaaactttacatagaagctgtttccaaattgggcaaaaattcccaacaagcactatggggatcttcagcagatgtcggtacctcactccttcaaattgtagaagtttggaacgaagtccagtctcaacaaatgaacattttgaaagcattctacgtggacatgatcgtgcccctggagacaaacctggagaaggacaccaaagtggtgcaagccgagcagaagcggttcttacagcagcacaagatcctctccgaatcttacagtaaggcggcatccaacatgaagaaacagagaaagaaacacaagagcaactccaaagccagcaacacagtcagcaacaaggagatcaaaacaatgcagattctagaagaagaaaagtcaaagctggatgcgttctgtgaacaaggattgaaaaatgctatgacacaggaacggcgtagatacgggttcgttctggaacggctctctagtctggctaaacactacctggcgtaccactcggctggcaacaccctgtatgagaagaacatagatgaatggattgaggtgaccaagaccagggaaatgctaccggacagtgtggaggcaatctgtgccaaacgattatggcaagtcaactattggccggatgaaggcgaggatgatctaacctccgtgacgtcacagttgaggaagtgcaaaagtatggatgcggcttctctcatcacagacatagcccgacctctgccccgcgcccgctctgattgcaacctgtctacaccaccacagagccccatgtctagaaatgattccagacctcagtcatcctgggaagctcctgttgccaaagctttatacgcgtacatgtcatctggagacaatcagcttaacttcatggagggagatatgattgctcttacaggtgatagaaataaaggctggcaatacggagaaaacctgagaacccaacagagtggctggttcccatgtgcttatgctgagcctatgctggaagaacaacacgtgacgccatctcacagaagatcagcttcgggtaaaagacctaccacttccccccctccccctcctgtaaccaagtttggtgatagtttgagataccagcgtcgagtcgggaattctacgttgagtattgacagaggctccccacctcagatagctccgccactggttccttcccttcacagtagcaacgacagtggctttggcaacgacccgcctgtggactactccgatgatgattgtaacaggaactcttcaagtacattgaagaagcctttatccaaacaaatgtcacttcaaccagaaagtgatgcgctcaaagacaaaggtgttgtaaagcgaaccaaatcctctctgtggaaattccgcaagaatgacgatgtccttgagggtatgagtctgtggagacatcggtctctagtggatgtcactaaggatgagaagacattcacctatgtgaaagatatcaacattgactatgacgaagataaagaaagcactctggtcaataacgatgatgaatcctgtattgtggtcacggacttcaaagtgaggaaagacgtcaaaggaaacgacttcagcctacgctcaaataagaaacctgatcctgacaatgaaatgaagaggaaatccatcagcagtaatatttatgaaatagagaaacctctcgtgaaaagagaacaagtgagacgatattatcgcgattatacaacagacaccgaggacaccaatgtcatgaaggtcaacgaagatttacgacgaaaaaaccgcgaaagtttcatcgaaaaagatatgtacgacatgaaactgaatgtcaacttggataagagatccaagaaagacataccgacgggcaccaaacaaaacaatgaacagatcaaaaaggacaaagcatccactctgcaacgtgaaacaccagatcggaaactagttgaagatcaaaagagaatttccagagaaatcgaacgcattcgtaacggagactttgttcaaaaaaccaataacaccatgaaagatgaagaccacaggaaaagtaacggctacaaaatgatgaacgggaacgcaaatcatagcatggatgacgtacggagaaagaatagagaatacagtgtagagagaggaagttatgataaactgaacaatatgtatgacgaggaggagagagacagacggaactatacacttgaaaaagaaagtgatataaagcaaaagtggaaagaaaattacgtcatgatgggatacaatgaaaggaatgacttgagtaaaaggtacagtgtagcaggagatagcaagatcaaaccagaatatattgaggacaacaggaggtacaaccctatcagcagcaagaaactaaacaacaggaaaattgagacagagagtgaagatgattggtatgactcctgggatgagaaatag

SEQ ID NO: 48 - RNA, 25 nt, synthetic construct
  source 1..25: mol_type = other RNA
agcagcttcttgcctctgattccac

SEQ ID NO: 49 - RNA, 23 nt, synthetic construct
  source 1..23: mol_type = other RNA
aatgcgaagctagcagtagccgc

SEQ ID NO: 50 - RNA, 26 nt, synthetic construct
  source 1..26: mol_type = other RNA
tccgtcgagttaactttagccaagcc

SEQ ID NO: 51 - RNA, 24 nt, synthetic construct
  source 1..24: mol_type = other RNA
acctacggtgacgattaaggcacc

SEQ ID NO: 52 - RNA, 22 nt, synthetic construct
  source 1..22: mol_type = other RNA
ttgcgttcctgctccctttgcc

SEQ ID NO: 53 - RNA, 27 nt, synthetic construct
  source 1..27: mol_type = other RNA
atcaactacgtcatagccagagtgacg

SEQ ID NO: 54 - RNA, 24 nt, synthetic construct
  source 1..24: mol_type = other RNA
ttacgtgccgttacaccggttacc

SEQ ID NO: 55 - RNA, 25 nt, synthetic construct
  source 1..25: mol_type = other RNA
cggtaagttaaggcgaccttgtccc

SEQ ID NO: 56 - RNA, 53 nt, synthetic construct
  source 1..53: mol_type = other RNA
gctggtcgcggctactgctagcttcgcatttcgcacaatgccttggagccatc

SEQ ID NO: 57 - RNA, 55 nt, synthetic construct
  source 1..55: mol_type = other RNA
agcgggcttggctaaagttaactcgacggaagcaattgcgaatcctatcgcaacg

SEQ ID NO: 58 - RNA, 53 nt, synthetic construct
  source 1..53: mol_type = other RNA
ctacgtcactctggctatgacgtagttgattcgcacaatgccttggagccatc

SEQ ID NO: 59 - RNA, 55 nt, synthetic construct
  source 1..55: mol_type = other RNA
gatgtcggtaaccggtgtaacggcacgtaaagcaattgcgaatcctatcgcaacg

SEQ ID NO: 60 - RNA, 54 nt, synthetic construct
  source 1..54: mol_type = other RNA
gctggtcgcggctactgctagcttcgcattctgtccatccaaccctggattgcc

SEQ ID NO: 61 - RNA, 56 nt, synthetic construct
  source 1..56: mol_type = other RNA
agcgggcttggctaaagttaactcgacggaaatggcgagaccaagaattgctctcg

SEQ ID NO: 62 - RNA, 54 nt, synthetic construct
  source 1..54: mol_type = other RNA
ctacgtcactctggctatgacgtagttgatctgtccatccaaccctggattgcc

SEQ ID NO: 63 - RNA, 56 nt, synthetic construct
  source 1..56: mol_type = other RNA
gatgtcggtaaccggtgtaacggcacgtaaaatggcgagaccaagaattgctctcg

SEQ ID NO: 64 - RNA, 24 nt, synthetic construct
  source 1..24: mol_type = other RNA
ctgtccatccaaccctggattgcc

SEQ ID NO: 65 - RNA, 26 nt, synthetic construct
  source 1..26: mol_type = other RNA
aatggcgagaccaagaattgctctcg

SEQ ID NO: 66 - RNA, 23 nt, synthetic construct
  source 1..23: mol_type = other RNA
tcgcacaatgccttggagccatc

SEQ ID NO: 67 - RNA, 25 nt, synthetic construct
  source 1..25: mol_type = other RNA
agcaattgcgaatcctatcgcaacg

SEQ ID NO: 68 - protein, 103 aa, synthetic construct
WRDQELLSPDLDPTNYWNYNRSRTMDQPNTYRLITRFREVFDFYTKKEGKTKVLMTEAYTTLDRTMDYYQFEGKPGAHMPFNFFFITHVSGRSPAKDYQKAIQ

SEQ ID NO: 69 - RNA, 231 nt, synthetic construct
  source 1..231: mol_type = other RNA
ttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatt

SEQ ID NO: 70 - RNA, 151 nt, synthetic construct
  source 1..151: mol_type = other RNA
gagaaggcagcttctttcatgacttcacccatgagggtttttgtctcgatgattttgctcaggatcatacggaatctcatctggagagcatcagctttcttcttaagcaaactgtgtcccttctgagcccccttgagacgggacttcatga

SEQ ID NO: 71 - RNA, 156 nt, synthetic construct
  source 1..156: mol_type = other RNA
aatctttcaaattaccactaaactctttcagcttcaatattggaagtttgcactgatactgagctatattacttccattagctgatacaaaactaccattttgatcatctggtgtatgtactgtctgactactctgaatcttactctcaaccagat

SEQ ID NO: 72 - RNA, 156 nt, synthetic construct; identical to SEQ ID NO: 71
  source 1..156: mol_type = other RNA

SEQ ID NO: 73 - RNA, 159 nt, synthetic construct
  source 1..159: mol_type = other RNA
tttcttctgaatgttgaaatgatgttgatggaattggatttgacatagtatcttctgggcttgcctgtattagtggtgttttttgtgcttctttatagggctttcttttcttggcagggggttgtaagtaagagggaaaagctgtaaaaactgaaggaa

SEQ ID NO: 74 - RNA, 159 nt, synthetic construct; identical to SEQ ID NO: 73
  source 1..159: mol_type = other RNA

SEQ ID NO: 75 - RNA, 463 nt, synthetic construct
  source 1..463: mol_type = other RNA
aatctttcaaattaccactaaactctttcagcttcaatattggaagtttgcactgatactgagctatattacttccattagctgatacaaaactaccattttgatcatctggtgtatgtactgtctgactactctgaatcttactctcaaccagatgagaaggcagcttctttcatgacttcacccatgagggtttttgtctcgatgattttgctcaggatcatacggaatctcatctggagagcatcagctttcttcttaagcaaactgtgtcccttctgagcccccttgagacgggacttcatgaaatctttcaaattaccactaaactctttcagcttcaatattggaagtttgcactgatactgagctatattacttccattagctgatacaaaactaccattttgatcatctggtgtatgtactgtctgactactctgaatcttactctcaaccagat

SEQ ID NO: 76 - RNA, 469 nt, synthetic construct
  source 1..469: mol_type = other RNA
tttcttctgaatgttgaaatgatgttgatggaattggatttgacatagtatcttctgggcttgcctgtattagtggtgttttttgtgcttctttatagggctttcttttcttggcagggggttgtaagtaagagggaaaagctgtaaaaactgaaggaagagaaggcagcttctttcatgacttcacccatgagggtttttgtctcgatgattttgctcaggatcatacggaatctcatctggagagcatcagctttcttcttaagcaaactgtgtcccttctgagcccccttgagacgggacttcatgatttcttctgaatgttgaaatgatgttgatggaattggatttgacatagtatcttctgggcttgcctgtattagtggtgttttttgtgcttctttatagggctttcttttcttggcagggggttgtaagtaagagggaaaagctgtaaaaactgaaggaa